SEQUENCE LISTING (2 sequences)

SEQ ID NO: 1 - protein, 9 aa, Artificial Sequence
  Synthetic polypeptide
GFLSKSLVF

SEQ ID NO: 2 - protein, 10 aa, Artificial Sequence
  Synthetic Polypeptide
RWKFGGFKWR

(19) United States
(12) Patent Application Publication
(10) Pub. No.: US 2024/0226292 A1
(43) Pub. Date: Jul. 11, 2024

(54) METHODS AND COMPOSITIONS FOR TARGETING OF ANTIGENS AND OTHER POLYPEPTIDES TO FIRST RESPONDER DENDRITIC CELLS

(71) Applicant: THE UNIVERSITY OF CHICAGO, Chicago, IL (US)

(72) Inventors: Aaron ESSER-KAHN, Chicago, IL (US); Rachel STEINHARDT, Chicago, IL (US); Bradley STUDNITZER, Chicago, IL (US); Peter DEAK, Chicago, IL (US)

(21) Appl. No.: 18/558,527

(22) PCT Filed: May 12, 2022
(86) PCT No.: PCT/US22/72297

§ 371 Date: Nov. 1, 2023

Related U.S. Application Data
(60) Provisional application No. 63/187,797, filed on May 12, 2021.

(51) Int. Cl. A61K 39/00; A61K 47/69; C07K 16/28; C12N 5/0784

(52) U.S. Cl. CPC A61K 39/4615; A61K 39/4622; A61K 47/6911; C07K 16/28; C12N 5/0639; A61K 2039/55555; A61K 2039/55561

(57) ABSTRACT

Disclosed herein are methods and compositions related to identification, isolation, and targeting of first responder dendritic cells (FRs). Aspects of the disclosure are directed to FR-targeting agents and methods for use of such agents, including methods for directing a diagnostic, imaging, or therapeutic molecule (e.g., an antigen) to FRs. The present disclosure includes pharmaceutical compositions comprising an FR-targeting agent and an antigen or polynucleotide encoding an antigen. Also disclosed are methods for stimulating an immune response comprising targeting an antigen to FRs.

### CROSS REFERENCE TO RELATED APPLICATIONS

This application claims benefit of priority to U.S. Provisional Application No. 63/187,797, filed May 12, 2021, which is hereby incorporated by reference in its entirety

### STATEMENT OF GOVERNMENT SUPPORT

This invention was made with government support under grant number AI124286 and AI147517 awarded by the National Institutes of Health. The government has certain rights in the invention.

### SEQUENCE LISTING

The instant application contains a Sequence Listing which has been submitted in ASCII format and is hereby incorporated by reference in its entirety. Said ASCII copy, created on May 12, 2022, is named ARCD_P0717WO_Sequence_Listing.txt and is 729 bytes in size.

### BACKGROUND

### I. Field of the Invention

Aspects of this invention relates to at least the fields of immunology and medicine.

### II. Background

Antigen presenting cells (APCs) initiate adaptive T and B cell response to pathogens by phagocytosing antigens and presenting antigen peptides on major histocompatibility type II molecules (MHCII). Given their importance for bridging the gap between adaptive and innate immunity, APCs are widely studied and are subdivided into various classical phenotypes of dendritic cells (DCs), B cells, Langerhans cells and macrophages.⁴ Of these, DCs are considered most critical for antigen presentation given their wide distribution in most dermal tissue, their migratory characteristics between dermal tissue and immune lymphoids and their high level of paracrine signaling which coordinates local immune responses.⁵ DCs are typically characterized by expression of CD11c and a high level of MHCII expression, however, DCs are also rather heterogenous and can be further divided into two main subtypes, a CD8+, XCR1+ subtype (cDC1) and a CD11+, CD172+ subtype (cDC2).⁶ Recent finding of sequencing, single-cell analysis, and microscopy have identified that within these subtypes, there are further divisions of distinct DCs substates. One substate of particular interest to us is the identification of a small (<5%) population of DC that can, in certain conditions, generate >70% of the inflammatory cytokine, TNFα, when simulated with a toll-like receptor (TLR) agonist, lipopolysaccharide (LPS).² These “super-secreting” cells also appear to be one of the important cells for initiating global activation of a wider population of heterogeneous APCs. Study of these cells so far has been limited, due to lack of surface markers or methods of isolation.⁸

Inherent heterogeneity in immune cell populations contributing to a small subset of cells with wide ranging effects on broader populations is well established, as cell specialization and amplification of responses are traditionally hallmarks of immune cells. For example, CD4+ T cells are known to coordinate neighboring cell immunity and propagate activation signals via IL-2 secretion when stimulated with antigen.⁹ Likewise, innate cells have shown heterogeneity. Neutrophils, generally described as “sentinel cells” due to their active homing to pathogens, have been shown to be more heterogeneous and have subsets that seems to be more active in migration to pathogens.¹⁰ Even in a different subset of DCs, notably plasmacytoid DCs (pDC), there is increasing evidence that a small population of interferon (INF) secreting cells regulate bulk populations of pDC activation.¹¹ Nevertheless, there is currently little direct evidence linking highly TLR reactive conventional DCs in a heterogeneous population and bulk activation of neighboring cells.

### SUMMARY

Aspects of the present disclosure fulfil certain needs in the field of immunology by providing compositions and methods for isolating and targeting first responder dendritic cells (FRs), disclosed herein as cells necessary to stimulate bulk innate cell TLR-mediated activation and useful for targeting of vaccine compositions and immunotherapeutics. Accordingly, embodiments of the present disclosure are directed to pharmaceutical compositions comprising an antigen and an FR-targeting agent, in some cases further comprising one or more adjuvants. Also disclosed are methods for stimulating an immune response comprising targeting an antigen to FRs. Further disclosed are methods for directing a molecule to FRs comprising providing the molecule operatively linked to an FR-targeting agent.

Embodiments of the present disclosure include methods for FR targeting, methods for stimulating an immune response to an antigen, methods for reducing an immune response to an antigen, methods for antigen targeting, methods for antigen delivery, methods for visualizing FRs, methods for isolating FRs, FR-targeting agents, polynucleotides, vectors, and pharmaceutical compositions. Compositions of the disclosure can include at least 1, 2, 3, or more of the following components: an FR-targeting agent, a polynucleotide encoding an FR-targeting agent, an antigen, a polynucleotide encoding an antigen, an adjuvant, a PRG2-binding agent, a DAP12-binding agent, a TMEM176A-binding agent, a TREM2-binding agent, a CLC5A-binding agent, a liposome, an exosome, a nanoparticle, a microparticle and an excipient. Any one or more of these components may be excluded from certain embodiments of the disclosure. Methods of the disclosure can include at least 1, 2, 3, or more of the following steps: obtaining a biological sample, isolating FRs, visualizing FRs, quantifying FRs in a sample, obtaining FRs from a subject, administering an FR-targeting agent to a subject, administering an antigen to a subject, and administering an adjuvant to a subject. Any one or more of these steps may be excluded from certain embodiments of the disclosure.

Disclosed herein, in some embodiments, is a pharmaceutical composition comprising (a) an antigen or a polynucleotide encoding an antigen; and (b) a first responder (FR)-targeting agent. Further disclosed herein, in some embodiments, is a method for stimulating an immune response to an antigen comprising administering to a subject an effective amount of a pharmaceutical composition comprising an antigen and a First Responder (FR)-targeting agent.

Also disclosed herein, in some embodiments, is a method for treating or preventing cancer in a subject comprising administering to the subject an effective amount of a pharmaceutical composition comprising a tumor antigen and a First Responder (FR)-targeting agent. In some embodiments, the method further comprises administering to the subject an additional cancer therapy. In some embodiments, the additional cancer therapy comprises chemotherapy, radiotherapy, immunotherapy, or a combination thereof.

Also disclosed herein, in some embodiments, is a method for treating or preventing an autoimmune or inflammatory condition in a subject comprising administering to the subject an effective amount of a pharmaceutical composition comprising a therapeutic agent and a First Responder (FR)-targeting agent. In some embodiments, the therapeutic agent is a cell killing agent. In some embodiments, the method further comprises administering to the subject an additional anti-inflammatory agent.

In some embodiments, the FR-targeting agent is conjugated to the antigen. In some embodiments, the FR-targeting agent is conjugated to a liposome comprising the antigen or polynucleotide encoding the antigen. In some embodiments, the FR-targeting agent is conjugated to a nanoparticle comprising the antigen or polynucleotide encoding the antigen. In some embodiments, the pharmaceutical composition further comprises an adjuvant. In some embodiments, the adjuvant is a TLR agonist. In some embodiments, the TLR agonist is a TLR9 agonist. In some embodiments, the TLR9 agonist is a CpG oligodeoxynucleotide (ODN). In some embodiments, the TLR agonist is a TLR7 agonist. In some embodiments, the TLR7 agonist is R848. In some embodiments, the FR-targeting agent is an agent capable of binding to a protein of Table 1. In some embodiments, the FR-targeting agent is a PRG2-binding agent. In some embodiments, the PRG2-binding agent is heparin. In some embodiments, the FR-targeting agent is a DAP12-binding agent. In some embodiments, the DAP12-binding agent is a polypeptide comprising SEQ ID NO:1. In some embodiments, the FR-targeting agent is a CD206-targeting agent. In some embodiments, the CD206-targeting agent is a polypeptide comprising SEQ ID NO:2. In some embodiments, the FR-targeting agent is a C9orfl35-targeting agent. In some embodiments, the FR-targeting agent is a TMEM176A-binding agent. In some embodiments, the FR-targeting agent is a TREM2-binding agent. In some embodiments, the FR-targeting agent is a CLC5A-binding agent. In some embodiments, the pharmaceutical composition further comprises an additional FR-targeting agent. In some embodiments, the FR-targeting agent is a PRG2-binding agent and the additional FR-targeting agent is a DAP12-binding agent. In some embodiments, the PRG2-binding agent is heparin and the DAP12-binding agent is a polypeptide comprising SEQ ID NO:1. In some embodiments, the FR-targeting agent is a PRG2-binding agent and the additional FR-targeting agent is a CD206-binding agent. In some embodiments, the PRG2-binding agent is heparin and the CD206-binding agent is a polypeptide comprising SEQ ID NO:2. In some embodiments, the FR-targeting agent is a DAP12-binding agent and the additional FR-targeting agent is a CD206-binding agent. In some embodiments, the DAP12-binding agent is a polypeptide comprising SEQ ID NO:1 and the CD206-binding agent is a polypeptide comprising SEQ ID NO:2. In some embodiments, the antigen is a tumor antigen. In some embodiments, the antigen is a viral antigen. In some embodiments, the antigen is a bacterial antigen. Also disclosed is a method for stimulating an immune response to an antigen comprising administering to a subject an effective amount of a pharmaceutical composition disclosed herein. In some embodiments, the subject is a mouse subject. In some embodiments, the subject is a human subject.

Throughout this application, the term “about” is used to indicate that a value includes the inherent variation of error for the measurement or quantitation method.

“Individual, “subject,” and “patient” are used interchangeably and can refer to a human or non-human.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments described herein in the context of the term “comprising” may also be implemented in the context of the term “consisting of” or “consisting essentially of.”

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary, Detailed Descriptions, Claims, and Brief Description of the Drawings.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1G. Identification of First Responder Cell State With Statistically Improbable Uptake of TLR coated MPs. FIG. 1A. Schematic figure illustrating the overarching hypothesis of the study. FRs uptake more TLR coated MPs, activating neighboring APCs via paracrine signaling. FIG. 1B. Flow cytometry of mouse spleenocytes incubated with MPTLR4 1:1 for 30 minutes. FRs are defined as top 5% of MP signal. FIG. 1C. Confocal microscopy images of BMDCs incubated with MPTLR4 1:1 for 30 minutes. Blue channel (DAPI), green channel (FITC MPTLR4), Red channel (NF-kB). Left image: wide field view of BMDCs and MPs (no NFkB staining). Middle Image: FR with NFkB staining, Right—nFR with NFkB staining. FIG. 1D. Percentage of MPs in FR cells. 100K BMDCs were incubated with 100K of varying MP formulations for 15 minutes, washed and then analyzed via ImageStream. The number of MPs uptaken by each cell was determined using the “spot counter” function on the IDEAS software and the percentage of all MPs in FRs was calculated. FIG. 1E. Spleen derived CD11c+ cells (sDCs) were incubated with MPs at a ratio of 1:1 for 15 mins, stained for CD11c+ and analyzed via Imagestream similar to part D. The number of cells was normalized to 10 k total MPs uptaken per sample. The number of cells with 1-6 MPs in these samples (actual) were compared to a random Poisson distribution (simulated). FIG. 1F. This analysis was repeated for RAW cells, BMDCs, and B cell depleted CD11c+ spleenocytes and a ratio of the actual/simulated was determined. FIG. 1G. Repeated analysis from FIG. 1F but varying the ratio of MPs to cells. All experiments were performed in biological triplicates, error bars indicate ±SD.

FIGS. 2A-2G. FRs are primarily central dendritic cell 2 (cDC2) class and are sufficient and necessary for activating populations of naïve BMDCs in vitro. FIG. 2A. Mouse Spleenocytes were isolated, incubated with MPTLR4 and phenotyped via Aurora flow cytometry. Representative flow plots for DC (CD11c+, MHCII hi) and/or FR (top 5% of MP signal). FIG. 2B. Graph showing percentage of FRs that were also cDC2 given various MP stimulation similar to part A. FIG. 2C. BMDCs incubated with Brefeldin A, (1 ug/mL) MPTLR-X (x=blank, 2, 4) were added to 100 k BMDCs. After 6 h, the TNFα levels were analyzed via ISX. FRs express higher levels of TNFα than nFRs. FIG. 2D. Three donor PBMC samples (10 million per donor) were differentiated into moDCs. 2 million moDCs per sample were incubated with 2 million MP-TLR4 or MP-Blank for 15 mins, washed and stained, then analyzed via ISX. The percent of MPs in FRs was calculated FIG. 2E. BMDCs were stimulated at a 1:1 ratio with MPTLR-4 for 15 min. The FRs and nFRs were isolated, washed, and resuspended at 1 million cells/mL in 10% HIFBS in RPMI for 1 h. The supernatant was collected and profiled via cytokine bead array (BD Biociences). FRs secreted 1406 pg/mL of TNFα—6.4 times more TNFα per cell when compared to nFRs. FIG. 2F. Naïve BMDCs were stimulated at 1:1 ratio with MPTLR4 and isolated the FRs and nFRs via FACS. After 0, 1, or 2 h incubation, FRs, nFRs, and unsorted BMDCs were added to 1 million naïve BMDCs in a 1:10 ratio. After 16 h, TNFα intensity was measured, and there was high TNFα expression in the bulk population when the FRs were added at 0 h incubation time. There was a background levels of TNFα expression when nFRs were added, regardless of timepoint. FIG. 2G. A similar experiment to F, but the naïve BMDCs were plated on the bottom section of a transwell assay and the MP-stimulated FRs, nFRs, and unsorted BMDCs were plated on top of the membrane. Similarly, naïve cells subjected to FRs express significantly higher levels of TNFα at 0 h incubation. All experiments were performed in biological triplicates, error bars indicate ±SD.

FIGS. 3A-3D. FRs are necessary and sufficient for triggering adaptive immune responses in vivo. FIG. 3A. Schematic of FR adoptive transfer experiment. BMDCs were incubated 1:1 with MPTLR4 for 30 minutes, washed and sorted into FR or nFR populations. Both hind leg footpads of a C57BL/6 mouse were injected with either 100K FRs, 1 million nFRs, 1 million unsorted MP incubated BMDCs, untreated BMDCs, CFA (positive control) or PBS (negative control). All samples except PBS contained OVA (10 ug per footpad). FIG. 3B. anti OVA IgG titers 14 days post injection. FIG. 3C. CD8+ T cells positive for a MHCI tetramer to the major MHCI epitope OVA 257-264. FIG. 3D. CD4+ T cells positive for a MHCII tetramer to the major MHCII epitope OVA 323-339. Each group had N=5 mice, error bars indicate ±SEM.

FIGS. 4A-4F. FR mRNA Analysis. BMDCs were incubated with MPTLR4 1:1 for 15 mins, washed and immediately sorted into FRs (top 5% of MP signal) and nFRs (bottom 90% of MP signal). The mRNA from these cells were isolated immediately off sorter (0 hr) or incubated at 37° C. for 0.5, 1, 2, or 4 hrs and isolated. cDNA was generated from the poly A mRNA using a commercially available kit from Illumina and sequenced. Genes were aligned and two-fold upregulation calculated by comparing to a non-treated BMDC control. Timecourse fold change of the following cytokines were plotted, TNFα (FIG. 4A), INFβ (FIG. 4B), CXCL1 (FIG. 4C) and IL-1β (FIG. 4D). FIG. 4E. Heatmap of log 2 fold change in the mRNA for genes with known antigen presentation function. Genes with pval <0.05 and 2 fold differential expression for at least one of the timepoints were included. FIG. 4F. BMDCs were incubated with varying MP formulations for 16 hrs with Brefeldin A treatment, washed and sorted into FRs and nFRs as in FIGS. 4A-4E and mRNA immediately isolated, transformed into cDNA, sequenced and genes aligned. A list of upregulated mRNA genes in FRs that correspond to surface receptors is shown in a heatmap. All experiments were performed in biological triplicates with significant upregulation determined only if the pval <<0.05.

FIGS. 5A-5G. Identification of Cell-Surface Markers of FR and Targeting of FRs with Liposomes. FIG. 5A. 1 million BMDCs were incubated with 1 million various MP formulations for 15 minutes, then stained for CD11c and either PRG2, DAP12 or TMEM176A and analyzed via flow cytometry. % Positive Ratio represents the ratio of PRG2, DAP12 or TMEM176A positive cells in the FRs divided by % positive cells in the nFR population of the CD11c+ cells. FIG. 5B. Similar to FIG. 5A but using mouse spleenocytes or (FIG. 5C) B cell depleted mouse spleenocytes or (FIG. 5D) RAW cells. FIG. 5E. 1% DAP12 peptide, 10% heparin-lipid loaded, DiD labeled (0.01%) 200 nm diameter liposomes were incubated at 10 uM total lipid concentration in combination with 1 million MPTLR4 with 1 million spleenocytes for 30 mins. Cells were washed and stained for CD11c and analyzed via flow cytometry. Plot is a representative example of gating strategy for CD11c+ cells to define Liposome+ and FRs. FIG. 5F. Graph demonstrating the percentage of cell population that were liposome+ for various MP formulations. FIG. 5G. Representative confocal images of BMDCs incubated with 10 uM total Lipid of targeted liposome and MPTLR7. Left—nFR (with no MP signal) with targeted liposome, Center—FRs incubated with 10 um of non-targed liposome, Right—FRs+ 10 uM of targeted Liposome. All experiments were performed in biological triplicates, error bars indicate ±SD.

FIGS. 6A-6F. In vivo validation of FR targeting in vaccination models. FIG. 6A. C57BL/6 mice were injected with Brefeldin A formulations (100 μg per mouse) and then 1 hr later injected with 100 μg OVA and 10 μg CpG on day 1. On day 14 both injections were repeated. Mice were sac'd on day 21 and their IgG titers analyzed. FIG. 6B. Lymph nodes from experiment in FIG. 7A were stained for T cell surface markers and with tetramers for the major OVA MHCI epitope (OVA 257-264) and the major MHCII epitope (OVA 323-339) and analyzed via flow. FIG. 6C. CpG and OVA were loaded into liposomes either targeted with DAP12/Hep (GpG FR-TL) or without (GpG NTL) and injected into C57BL/6 mice and compared to a PBS control or free CpG OVA equivalent (100 μg OVA and 10 μg CpG per mouse). 14 days later anti-OVA IgG titers were measured. FIG. 6D. R848 (10 μg per mouse) replaced CpG in a repeat of experiment from FIG. 7A. Mice were injected on day 1 and day 14 and then sac'd and anti-OVA IgG titers were measured on day 21. CD45+ CD3+ CD4+ MHCII tetramer+ and CD45+ CD3+ CD8+ MHCI tetramer+ cells are shown. FIG. 6E. Aurora analysis similar to FIG. 6C of lymph nodes of mice from FIG. 6D. parts a-e, N=5, error bars indicate ±SD and all injections were i.p. FIG. 6F. E7-OVA tumor model. C57BL/6 mice (N=5) were injected with 2×10⁵ E7-OVA tumor cells in the left flank s.c. on day 1 and tumors measured 3× per week. On day 7 and day 10, mice were injected i.p. with same R848/OVA formulations from FIG. 6B. Tumors were tracked until day 30. Error bars indicate ±SEM. *indicates p<0.05, **indicates p<0.01.

FIGS. 7A-7E. TLR conjugated MP Chemistry. FIG. 7A. Chemistry schematic of the thiol-maleimide chemistry for TLR agonist conjugation FIG. 7B. SEM of MPTLR4, scale bar=500 nm. FIG. 7C. RAW Blue assay of TLR conjugated MP (grey) with free agonist (black) for comparison, note that RAWs do not express TLR5. FIG. 7D. HEK-mTLR5 assay for free flagellin (FLA) and flagellin conjugated MPs. FIG. 7E. Calculation of the number of molecules per MP via BCA assay using free agonists as standard curves. Error bars, ±SD of biological triplicate assays.

FIGS. 8A-8B. A small population of innate immune cells uptake a high number of MPs. FIG. 8A. 1 million of various cells (BMDCs, sDCs RAWs) were incubated with MPTLR4 at a 1:1 ratio for 15 mins or BMDCs only (Blank BMDCs) then analyzed via flow cytometry. FIG. 8B. Zoomed in picture of right side of graph of A, showing skewing towards high MP signal.

FIGS. 9A-9B. BMDCs isolated are all DCs. BMDCs were isolated and treated according to methods. At day 7, BMDCs were analyzed via flow cytometry. FIG. 9A. Most cells were CD11b/c+ and MHCII hi, indicating that they are BMDCs. FIG. 9B. Additionally most cells (60%) were immature DCs (GM-DC, FTL-3+, MCSF+) while only (40) were mature (GM-DN, FTL-3−)

FIGS. 10A-10C. Heterogeneous populations of innate immune cells have small population of cells that uptake more MPs than a random distribution. 100 k cells were incubated with 100 k MPTLR4 for 15 mins, washed and analyzed via ISX. The number of MPs per cell were plotted for BMDCs (FIG. 10A), Spleen DCs (FIG. 10B) and RAWs (FIG. 10C).

FIGS. 11A-11B. FRs uptake a statistically significantly higher number of MPs per cell. BMDCs (FIG. 11A) or RAWs (100 k cells) (FIG. 11B) were incubated with 100 k MPs and analyzed via ISX. The FRs (the top 5% of the FITC signal) were grouped and their average number of MPs uptaken were compared to a standard Possion distribution for the top 5%. * p<0.05, Error bars, ±SD of biological triplicate assays.

FIG. 12. MP:Cell ratios change percentage of MP uptaken in FR populations. 1 million BMDCs were incubated with MPTLR4 at various cell to MP ratios for 15 mins, washed and then analyzed via Imagestream analysis (100K cells analyzed). The total number of MPs uptaken were calculated and compared to the MPs uptaken in the FR population (top 5% of MP signal). Error bars are SD of triplicate biological experiments.

FIG. 13. Spleenocyte/lymph node cDC gating strategy. Spleenocytes were gated on live cells via size and on single cells and then gated on CD45. CD45 spleeocytes were then gated against CD19 and Ly6C. CD19− and LyC6− cells were gated into CD11c+, MHCII hi populations (DCs). The DCs were sorted into two groups, XCR1+, CD8a+ (cDC1) and CD11b+ CD8a+ (cDC2)

FIG. 14. FRs are primarily cDC2 subtype in foodpad skin cells. C57BL/6 mice injected via footpad with 1 million of varying MP formulations. Mice were sacrificed 1 hr later, footpads disaggregated, stained for surface markers and analyzed via aurora analysis. N=3 mice per group.

FIG. 15. Gating strategy for moDCs. 2 million moDCs were incubated with 2 million TLR4-MPs for 15 mins, washed, stained and analyzed via Aurora. The gating strategy is shown for identifying DCs.

FIGS. 16A-16B. moDCs have FR populations. moDCs treated from figure S-9 were analyzed for MP uptake. FIG. 16A. Representative flow plot of MP uptake showing that the distinct FR population of about 1%. FIG. 16B. Analysis of three different patient samples for FR % with uptake of MP-TLR4 or MP-Blank. TLR4−N=3, Blank—N=2

FIG. 17. moDC FRs overexpress the immaturity marker DC-SIGN. Samples for S-10 were analyzed for DC-SIGN expression in FR population vs all DCs.

FIG. 18. A small percentage of cells have large increases in TNFa expression. 10 k BMDCs were incubated on a coverslip and then treated with MPs or free LPS (1 ug/mL) for 15 mins, then washed an incubated with brefeldin A for 4 hrs (0.5 ug/mL). Cells were then fixed, permeabilized and stained for TNFa (red) and the nucleus (Blue). Note that TNFα expression is concentrated in a small number of cells.

FIG. 19. FR IL-1β Secretion Analysis. FRs and nFRs express baseline IL-1β after 1 h. BMDCs were incubated at 1:1 ratio with MP-TLR4 for 15 min. The FRs and nFRs were isolated via FACS, washed, resuspended in 10% HIFBS in RPMI at a concentration at 1 million cells per mL. After 1 h the supernatant was collected and the IL-1β was measured by ELISA (Biolegend).

FIG. 20. TLR conjugated MPs are uptaken via lysosomes in BMDCs. 100 k BMDCs were incubated with 100 k MPTLR7 for 15 mins, washed and then incubated for 1 hr at 37° C. Cells were then washed, fixed and stained for LAMP-1 (in purple) and stained with Hoest (blue) for the nucleus. Cells were then analyzed via confocal microscopy. Images show nucleus+MP (left), nucleus +LAMP (middle) and combined images (right). Red arrow points to FRs, showing overlap of LAMP−1 and MP.

FIGS. 21A-21B. FRs are necessary for global APC CD40 expression. FIG. 21A. Naïve BMDCs were stimulated at 1:1 ratio with MPTLR4 and isolated the FRs and nFRs via FACS. After 0, 1, or 2 h incubation, FRs, nFRs, and unsorted BMDCs were added to 1 million naïve BMDCs in a 1:10 ratio. After 16 hr, CD40 expression was measured via flow. FIG. 21B. Same experiment as C, but the naïve BMDCs were plated on the bottom section of a transwell assay and the MP-stimulated FRs, nFRs, and unsorted BMDCs were plated on top of the membrane. All experiments were performed in biological triplicates, error bars indicate ±SD. All experiments were performed in biological triplicates, error bars indicate ±SD.

FIGS. 22A-22C. nFRs can be re-stimulated after several hours and trigger global APC activation. FIG. 22A. Schematic of experiment. FIGS. 22B-22C. 100 k nFR (sorted out from studies shown in FIGS. 3C-3D) were allowed to incubate for various timepoints, then restimulated with MPTLR4 for 15 mins (1:1), washed and then added to naïve BMDCs (10 naïve:1 nFRs) then tested for CD40 expression (FIG. 22B) or TNFα (FIG. 22C). All experiments were performed in biological triplicates, error bars indicate ±SD.

FIG. 23. FRs have increased MHIC expression and antigen presentation in vitro. 1 million BMDCs were incubated with OVA-AF647 (10 ug/mL) for 3 hrs with 1 million MPs then stained for MHCII and MHCI-SIINFEKL. Fold increase calculated from CD11c+ cells FR/nFRs. All experiments were performed in biological triplicates, error bars indicate ±SD.

FIGS. 24A-24E. Adoptive Transfer of DiI labeled BMDCs into mouse footpad show that BMDCs migrate to popliteal lymph node. 1 million BMDCs were labeled with DiI and then incubated with MP-TLR4 or not for 15 mins, then injected into mouse footpads. Popliteal lymph nodes were extracted 24 hrs later and analyzed via flow cytometry. FIG. 24A. Percent CD80 positive cells of all lymphocytes. FIG. 24B. Time study of CD80% cells days post MP-TLR4 treated BMDC injection. FIG. 24C. % of spleenocytes that are DiI positive. FIG. 24D. Percentage of cells that are MP+ and DiI+. FIG. 24E. IL-6 levels in blood 1 hr post BMDC injection Error bars are ±SD of a group of 3 mice.

FIGS. 25A-25C. Adoptively Transferred FRs Trigger Adaptive T and B cell responses in vivo. Lymphocytes taken from the experiment in FIG. 4 were analyzed via flow cytometry. Activated CD4+ T cell (FIG. 25A), Activated CD8+ T cells (FIG. 25B) and Activated B cells (FIG. 25C). Error bars are ±SEM of a group of 5 mice.

FIG. 26. FR candidate protein testing. 1 million of various cells were incubated with MPs, washed and then analyzed via flow cytometry. Fold increase calculated from CD11c+ cells FR/nFRs. All experiments were performed in biological triplicates, error bars indicate ±SD.

FIG. 27. FRs do not have an increase in B220 or CD19. 1 million of various cells were incubated with MPs, washed and then analyzed via flow cytometry. Fold increase calculated from CD11c+ cells FR/nFRs. All experiments were performed in biological triplicates, error bars indicate ±SD.

FIGS. 28A-28E. FR Targeting liposome characterization. FIG. 28A. DAP12 targeting peptide was conjugated to a palmitic acid tail, purified via HPLC and characterized via MS. FIG. 28B. DAP12 lipid was loaded at 0.01, 0.1 and 1% and with 0.1% DiD and incubated with BMDCs for 15 mins, washed and then incubated with MPTLR4 (1:1) for 15 mins, washed and analyzed via flow cytometry. FIG. 28C. Chemistry of heparin sulfate-lipid. FIG. 28D. HPLC analysis of DAP12/heparin lipid. Liposomes were synthesized with DAP12 peptide (1%) and heparin at (10%) (DAP/Hep) and then dialyzed with 10 kDa filters in PBS for 24 hrs. Loading was calculated against a standard curve via HPLC. FIG. 28E. DLS analysis of 200 nm liposomes. All experiments were performed in biological triplicates, error bars indicate ±SD.

FIGS. 29A-29C. FR targeting liposomes are most effective with both DAP12 targeting and heparin sulfate-lipid. DiD loaded (0.1%) liposomes were prepare with DAP12 targeted peptide-lipid at 1% (FIG. 29A) heparin-lipid at 1% (FIG. 29B) or both (FIG. 29C). BMDCs were incubated with liposomes for 15 mins, washed and then incubated with MPs (1:1) for 15 mins then washed and analyzed via flow. All experiments were performed in biological triplicates, error bars indicate ±SD.

FIGS. 30A-30B. TLR agonist loaded FR targeting liposomes characterization. 200 nm liposomes were synthesized at 10 mM total lipid and loaded with OVA (1 mg/mL) and OVA plus CpG (0.2 mg/mL), R848 (0.2 mg/mL) or Brefeldin A (1 mg/mL), dialyzed for 24 hrs with a 10 kDa membrane against PBS. HPLC analysis of loading (FIG. 30A) and DLS analysis of liposomes (FIG. 30B).

FIG. 31. Brefeldin A loaded liposomes suppress global DC activation only when targeted to FRs. 500 k BMDCs were incubated with liposomes with or without targeted loaded with brefeldin A (loaded at 1% total lipid) the incubated for 1 hr and washed and incubated with 1 ug/mL of R848 for 16 hrs, then tested for CD40 expression via flow cytometry. Error bars represent ±SD of biological triplicates

FIGS. 32A-32B. Aurora Analysis of Lymph nodes from brefA in vivo experiment. Lymph nodes from FIG. 7E were further analyzed. FIG. 32A. percent of all lymph cells that were CD45+, CD3+/CD45+ (T cells), CD4/CD3/CD45+ or CD8/CD3/CD45+. FIG. 32B. T cells were further divided into Activated (CD25/CD44/CD69+), effector memory (Tem, CD45+/CD62L−), resident memory (Trm, CD27−/CD62L−/CD44+/CD69+), central memory (Tcm, CD27+/CD28+/CD44+/CD127+/CD62L+) or stem cell memory (Tscm, CD27+/CD28+CD62L+/CD127+/CD69−). Error bars represent ±SD of 5 mice per group.

FIGS. 33A-33B. Intracellular Staining analysis of Brefeldin A in vivo experiment spleenocytes. Spleenocytes were incubated for 12 hrs with monensin and either the major MHCI epitope (FIG. 33A) or the major MHCII epitope (FIG. 33B) of OVA (1 μg/mL) and then stained for CD4/8 and IL-4/INFγ using ICS procedure described in the methods). Error bars represent ±SD of 5 mice per group.

FIG. 34. Systemic TNFα levels in vivo after CpG/OVA liposome injection. 1 hr after 100 ul of liposome formulation or free CpG/OVA equivalent (10 μg/100 μg) or a PBS control serum samples were taken and analyzed via CBA. Error bars represent ±SD of 5 mice per group.

FIGS. 35A-35B. Aurora Analysis of Lymph nodes from R848 in vivo experiment. Lymph nodes from FIG. 7C were further analyzed. FIG. 35A. Percent of all lymph cells that were CD45+, CD3+/CD45+(T cells), CD4/CD3/CD45+ or CD8/CD3/CD45+. FIG. 35B. T cells were further divided into Activated (CD25/CD44/CD69+), effector memory (Tem, CD45+/CD62L−), resident memory (Trm, CD27−/CD62L−/CD44+/CD69+), central memory (Tcm, CD27+/CD28+/CD44+/CD127+/CD62L+) or stem cell memory (Tscm, CD27+/CD28+CD62L+/CD127+/CD69−). Error bars represent ±SD of 5 mice per group.

FIGS. 36A-36B. Intracellular Staining analysis of R848 in vivo experiment spleenocytes. Spleenocytes taken from mice in FIG. 7B were incubated for 12 hrs with monensin and either the major MHCI epitope (FIG. 36A) or the major MHCII epitope (FIG. 36B) of OVA (1 μg/mL) and then stained for CD4/8 and IL-4/INFγ using ICS procedure described in the methods. Error bars represent ±SD of 5 mice per group.

FIG. 37. Survival Curve for E7-OVA tumor experiment. Mice were sacrificed when tumor was >20 mm in any direction.

FIGS. 38A-38C. FRs exist in human dendritic cell populations. 1 million monocyte derived dendritic cells differentiated from human PBMCs were incubated with 1 million MPs for 15 mins, then fixed in 2% PFA for 15 mins, stained with antibodies for various cell receptors for 30 mins on ice and analyzed via flow cytometry. (FIG. 38A) Gating strategy for human moDCs. (FIG. 38B) Analysis of % FR populations (FIG. 38C) Similarly treated moDCs were analyzed via ImageStream cytometry to determine the number of MPs uptaken per cell. Then the percentage of all MPs uptaken present in the FR population calculated. N=3 per MPTLR4 or N=2 for MP Blank.

FIGS. 39A-39E. FRs are more likely actively dividing. (FIG. 39A) 1 million BMDCs were incubated with 1 million MP-TLR4 for 15 mins, washed and stain with Hoest for nuclear stain (0.1 ug/mL) and anti-CD11c antibody. Cell were analyzed via image stream analysis, both FR (left) and all CD11c+ cells for nuclear stain, indicating that most BMDCs FRs are in G2 phase. (FIG. 39B) Graph of imagestream data from FIG. 39A from an N=3 showing that most BMDC FRs are in G2 phase. (FIG. 39C) 1 million BMDCs were similarly treated as in FIG. 39A, except some samples were treated with 10 nM Nocodazole for 20 hrs prior to MP-TLR4 treatment. NT=non-treated, Noco=Nocodazole treated. (FIG. 39D) Similar to FIG. 39C, BMDCs were treated with MP-TLR7 or Blank MPs (1:1 particles/cells) for 15 mins, then tested via imagestream. Total MP uptake per 100 k BMDCs were calculated for either NT or Noco treated samples. (FIG. 39E) Applying the same FR cutoff from FIG. 39A, the total % of FRs for Noco or NT was calculated, showing increases in FR population when locked in G2 phase using Noco as a cell cycle inhibitor. N=3, Error bars indicate +/−SD.

FIGS. 40A-40C. FRs isolated from mouse spleens exist more in S phase. 10 million splenocytes were isolated, allowed to rest for 2 hrs in splenocyte media (5% HIPBS in RPMI+1× BME). Cells were incubated with MP-TLR4 for 15 mins, washed and stained. Cells were stained for various cell surface markers to identify DCs (CD45+, CD3−, CD19−, CD11c+, MHCI hi), cells were then fixed and permabilized then stained for nuclear stain (PI) and Ki-67. FRs were identified via hi uptake of MPs. Gating strategies for cell cycle stage of (FIG. 40A) all DCs and (FIG. 40B) FR-DCs. (FIG. 40C) Plotted data of cell cycle phase for all DCs and FR-DCs with N=3.

FIGS. 41A-41B. BMDC FRs have increased markers of G2 phase. 200 k BMDCs either treated with 2 nM Nocodozole for 16 hrs prior or not were treated with MPs for 15 mins, fixed and then stained for (FIG. 41A) survivin or (FIG. 41B) cyclin B. Cells tested for cyclin B were permeabilized prior to staining. Cells were then analyzed via flow cytometery. N=3, Error bars are +/− of SD.

FIGS. 42A-42D. Increasing G2 phase in vivo increases vaccine response. Liposomes were loaded with two agents to block cell cycle (Cytoclastin D, CytoD, which blocks cells in G1 phase and Nocodozole, Noco, which blocks cells in G2 phase) as well as flagellin(FLA) as an TLR agonist. Mice were injected with 200 uL of 10 mM total lipid liposomes containing either (1) PBS only, Blank (2) FLA, 1 ug total per injection (3) FLA and 100 ug CytoD or (4) FLA+20 ug Noco. Mice were injected i.p with liposomes on day 1, then injected with 100 ug OVA/10 ug CpG (free formulation) on day 2 also i.p. Sera was collected via cheek bleed 1 hr after OVA/CpG injection. Mice were then sac'd on day 10 to observe anti-OVA Ig. Serum levels 1 h after injection for (FIG. 42A) IL-4, (FIG. 42B) MCP-1 and (FIG. 42C) TNF-a. (FIG. 42D) Total anti-OVA Ig on day 1. N=5 mice per group.

FIGS. 43A-43F. FRs mRNA analysis. 100 million mouse splenocytes or 2 million human monocyte derived DCs (moDCs) were incubated with 1:1 cell to MP of either MP-blank or MP-TLR4 and stained with CD11c for 15 mins, washed and sorted into CD11c+ cells then into MP-(nFRs) or MP hi populations (FRs). Controls with PBS added or LPS (1 ug/mL for 16 hrs prior) and sorted into CD11c+ populations. Cells were sorted directly into mRNA extraction media, cDNA libraries prepared and sequenced via a NextSeq550. mRNA was analyzed using PCA analysis for gene expression for (FIG. 43A) mouse DCs and (FIG. 43B) human moDCs. (FIGS. 43C-43F) Plot of Log 2Fold gene change by p values for (FIG. 43C) mouse splenocyte MP-TLR4 FRs vs untreated, (FIG. 43D) moDC MP-TLR4 FRs vs untreated (FIG. 43E) mouse treated splenocyte DCs vs untreated and (FIG. 43F) splenocyte nFR DCs vs untreated. (N=3)

FIGS. 44A-44D. Gene Set Enrichment Data for FRs. Sequencing data from FIG. 6 was analyzed for gene set enrichment using DEG pathway analysis for (FIG. 44A) mouse splenocyte FR DCs vs untreated or (FIG. 44B) human moDCs FR DCs vs untreated and using KEGG pathway analysis for (FIG. 44C) mouse splenocyte FR DCs vs untreated or (FIG. 44D) human moDCs FR DCs vs untreated

FIGS. 45A-45B. FRs express unique surface proteins. (FIG. 45A) 1 million BMDCs were incubated with various MP formulations at 1:1 cell to MP for 15 mins, washed and quickly stained on ice for cell surface markers, then the fold increase for FRs over all DCs (CD11c+) calculated. (FIG. 45B) Experiments shown in FIG. 45A were repeated with 10 million mouse splenocytes per sample. (N=3).

FIGS. 46A-46D. FRs can be isolated via antibody expression. 100 million mouse splenocytes were incubated with FR abs (PRG2, CD206 and C9orfl35) and other surface markers on ice for 30 mins, fixed, permabilized, and treated with Ki-67 antibodies and Pi and then analyzed via flow cytometry. (FIG. 46A) representative gating strategy for FRs via Abs. Note that cells were already gated on live, single, CD11c+ cells. (FIG. 46B) Cell cycle analysis of FRs identified via Abs. (FIG. 46C) FR surface marker RFU for cells in various cell cycle phases. (FIG. 46D) In a separate experiment, FRs were sorted via Abs similar to the studies shown in FIG. 46A and incubated in a 1:100 dilution with either naïve splenocytes or 1:10 with BMDCs. nFRs (i.e. CD206−, PRG2−, C9orfl35 DCs) were incubated 1:10 with splenocytes or 1:1 with BMDCs as a control. Cells treated with 1 ug/uL LPS were also used as a control. For each sample (except blank and LPS controls) sorted cells were treated with brefeldin A and LPS (1 ug/mL) for 15 mins and washed prior to incubation with naïve cells. N=3.

FIGS. 47A-47B. FRs isolated via antibodies have similar increases in inflammatory cytokines as MP isolated FRs. 10 million splenocytes were incubated with LPS (1 ug/mL) and brefeldin A for either 1 hr or 15 mins, then incubuated with MP-blank for 15 mins, then stained on ice and fixed. Cells were analyzed for intracellular cytokine expression of either INFb and TNFa. (FIG. 47A) Fold increase in INFb over all splenocytes MFI for FRs identified via Abs or MP uptake and for CD11c+ of those groups. (FIG. 47B) TNFa+, INFb+ cells in similar groups to FIG. 47A. N=3

FIGS. 48A-48B. FR targeting peptides. A peptide targeting CD206 as described in a study by Ghebremedhin et al (Ghebremedhin A, Salam AB, Adu-Addai B, et al. Preprint. bioRxiv. 2020;2020.07.27.218115; incorporated herein by reference), having sequence RWKFGGFKWR (SEQ ID NO:2), was synthesized using the same lipid-peptide display technique as shown previously and incorporated into 200 nm liposomes at 1% total lipid. (FIG. 48A) 200K BMDCs were incubated with liposomes at either 1 or 10 uM total lipid concentration for 15 mins, then incubated with MP-TLR4 (200K) for 15 mins, washed and stained. The liposomal RFU is plotted for various formulations. MMR=CD206 targeting lipid at 1% total lipid, DAP12=DAP12 targeting lipid at 1% total lipid, Hep=heparin-lipid conjugate at 10% total lipid. (FIG. 48B) similar to part A, but using 2 million splenocytes and gating on all CD11c+ cells.

### DETAILED DESCRIPTION

The present disclosure is based, at least in part, on the surprising discovery of a rare and temporal dendritic cell state, disclosed herein as first responder dendritic cells (also “first responders” or “FRs”), and methods for isolation and targeting of such cells. As described herein, immune responses (e.g., IgG titers and CD8 responses) can be modulated by targeting upregulated cell surface proteins on FRs, such as DAP12, PRG2, CD206 (MMR), C9orfl35, and others. Vaccine compositions and methods for use in targeting FRs for immune modulation are disclosed.

### I. First Responders and Targeting Agents

Aspects of the present disclosure relate to first responder dendritic cells (FRs), also referred to as “FRs,” “first responders,” and “first responder cells,” which terms are used interchangeably herein. A single first responder dendritic cell may also be referred to here in a “first responder,” a “first responder cell,” or an “FR,” which terms are used interchangeably.

Without wishing to be bound by theory, FRs are understood to be a transcriptionally distinct cellular substate of dendritic cells belonging predominantly to the cDC2 subset. FRs have increased sensitivity to TLR signaling and disseminate global antigen presenting cell (APC) activation via paracrine signaling. FRs exist in a temporally controlled state, with cells maintaining a high activation state for hours before returning to a “non-FR” state. As disclosed herein, FRs are necessary to simulate bulk innate cell TLR-mediated activation. FRs can be isolated using, for example, labeled (e.g., fluorescently labeled, labeled with a retrieval tag, etc.) TLR agonist-conjugated microparticles. As disclosed herein, FRs may be identified and targeted using one or more of a number of protein markers. Example proteins markers that may be used for identifying and/or targeting FRs are provided in Table 1. Any one or more (e.g., 1, 2, 3, 4, or 5) of the proteins of Table 1 may be used in methods of the disclosure for identification, detection, targeting, or otherwise manipulating FRs.

TABLE 1
FR identifying markers
Protein Marker

Rcbtb2
Tyrobp (PRG2)
DAP12
CLC5A
TREM2
Cd300lb
F5
Ephx1
Entpd1
Hnmt
Lpar6
Serhl
Adgre5
Adcy7
Clec4a2
Gm8730
Npm1
Lyz1
Rpl5
Pck2
Gm13394
Msmo1
Fxyd2
Rps6
Rpl18a
Eef1b2
Qpct
Pcna
Cdk1
Cd300ld
Rnf13
Rpl12
Sgpp1
Rpl4
Slc30a5
Eif4b
Ank
Tmco1
Cd72
Rpl27a
Ogt
Kcnj2
Paics
Sesn1
Gm9625
Fcgr3
Slc50a1
Cd200r1
Txnip
Tmem273
Gm15772
Arhgap18
Rgs18
Prrc1
Stmn1
CD37
CD79a
SIGLEC5
VSIG4
CD72.1
FCGR3A (CD16)
C9orf135
CXCR1
HLA-DQA1
CD206 (MMR)

Aspects of the disclosure are directed to compositions and methods comprising FR-targeting agent. An “FR-targeting agent,” as used herein, describes any molecule capable of preferentially binding (also “specifically binding”) to an FR (e.g., a protein on the surface of an FR, an internal protein of an FR, a lipid of an FR, etc.) as compared to other types of immune cells. In some embodiments, an FR-targeting agent binds to an FR with at least, at most, or about 20%, 30%, 40%, 50%, 60%, 70%, 80%, 90%, 100%, 150%, 200%, 300%, 400%, 500%, or 1000% greater affinity, or more, compared to any other immune cell.

In some embodiments, an FR-targeting agent is a molecule capable of specifically binding to a protein preferentially expressed by an FR (e.g., a protein of Table 1, Table 2, or Table 3). In some embodiments, an FR-targeting agent is a molecule capable of specifically binding to PRG2 (i.e., a “PRG2-binding agent”), DAP12 (i.e., a “DAP12-binding agent”), TMEM176A (i.e., a “TMEM176A-binding agent”), TREM2 (i.e., a “TREM2-binding agent”), CLC5A (i.e., a “CLC5A-binding agent”), CD206 (i.e., a “CD206-binding agent”), or C9orfl35 (i.e., a “C9orfl35-binding agent”). In some embodiments, the FR-targeting agent is a PRG2-binding agent. In some embodiments, the FR-targeting agent is a DAP12-binding agent. In some embodiments, the FR-targeting agent is a TMEM176A-binding agent. In some embodiments, the FR-targeting agent is a TREM2-binding agent. In some embodiments, the FR-targeting agent is a CLC5A-binding agent. In some embodiments, the FR-targeting agent is a CD206-binding agent. In some embodiments, the FR-targeting agent is a C9orfl35-binding agent.

In some embodiments, an FR-targeting agent is an antibody or antibody-like molecule configured to bind to a protein preferentially expressed by an FR (e.g., a protein of Table 1). In some embodiments, an FR-targeting agent is an antibody or antibody-like molecule configured to bind to PRG2, DAP12, TMEM176A, TREM2, CLC5A, CD206 (also “mannose receptor” or “MMR”) or C9orfl35.

In some embodiments, an FR-targeting agent is a polypeptide configured to bind to a protein preferentially expressed by an FR (e.g., a protein of Table 1). In some embodiments, an FR-targeting agent is a polypeptide, such as an antibody, antibody-like molecule, or other binding agent, configured to bind to PRG2, DAP12, TMEM176A, TREM2, CLC5A, CD206, or C9orfl35. In some embodiments, the FR-targeting agent is a PRG2-binding polypeptide. In some embodiments, the PRG2-binding polypeptide is heparin. In some embodiments, the FR-targeting agent is a DAP12-binding polypeptide. In some embodiments, the DAP12-binding polypeptide is a peptide having amino acid sequence GFLSKSLVF (SEQ ID NO:1). In some embodiments, the FR-targeting agent is a TMEM176A-binding polypeptide. In some embodiments, the FR-targeting agent is a TREM2-binding polypeptide. In some embodiments, the FR-targeting agent is a CLC5A-binding polypeptide. In some embodiments, the FR-targeting agent is a CD206-binding polypeptide. In some embodiments, the CD206-binding polypeptide is a peptide having amino acid sequence RWKFGGFKWR (SEQ ID NO:2). In some embodiments, the FR-targeting agent is a C9orfl35-binding polypeptide.

FR-targeting agents of the present disclosure may be used to target one or more additional agents to FRs. In some embodiments, an FR-targeting agent is used to deliver an imaging agent to FRs for visualization. For example, an FR-targeting agent may be covalently linked, non-covalently linked, or otherwise associated with an imaging agent and administered to a cellular sample. In some embodiments, an FR-targeting agent is used to deliver a therapeutic agent to FRs. A therapeutic agent may be, for example, a cell killing agent (i.e., an agent capable of killing a cell). Various cell killing agents are recognized in the art. As disclosed herein, delivery of a cell killing agent to FRs may reduce the severity of an immune response (e.g., an adaptive immune response and/or an innate immune response), thereby treating an autoimmune or inflammatory condition. Accordingly, aspects of the present disclosure are directed to methods for treating or preventing an autoimmune or inflammatory condition in a subject comprising administering to the subject a composition comprising an FR-targeting agent and a cell killing agent.

In some aspects, a combination of two or more FR-targeting agents are used to target an FR. Any combination of the proteins of Table 1 may be used for FR targeting. In some aspects, disclosed are compositions comprising a PRG2-targeting agent (e.g., heparin) and a CD206 targeting agent (e.g., a polypeptide having sequence RWKFGGFKWR (SEQ ID NO:2)), as well as methods of use thereof. In some aspects, disclosed are compositions comprising a DAP12-targeting agent (e.g., a polypeptide having SEQ ID NO:1) and a CD206 targeting agent (e.g., a polypeptide having SEQ ID NO:2), as well as methods of use thereof. In some aspects, disclosed are compositions comprising a PRG2-targeting agent (e.g., heparin) and a DAP12 targeting agent (e.g., a polypeptide having SEQ ID NO:1), as well as methods of use thereof.

A therapeutic agent may be, for example, an antigen such as a viral antigen, bacterial antigen, tumor antigen, etc. As disclosed herein, delivery of an antigen to FRs may stimulate an immune response against the antigen. Such an immune response may be, for example, an immune response against a protein of an infectious agent such as a virus or bacteria. Alternatively, such an immune response may be an immune response against a tumor antigen, thereby improving the efficacy of a cancer immunotherapy. Such targeted delivery may, for example, allow for use of less antigen compared to either untargeted delivery or even targeted delivery to other immune cells (e.g., antigen presenting cells). Accordingly, aspects of the disclosure are directed to vaccine compositions comprising an antigen and an FR-targeting agent. The antigen may be covalently or non-covalently linked (i.e., conjugated) to the FR-targeting agent. In other embodiments, the antigen may be conjugated to a liposome or nanoparticle, where the liposome or nanoparticle comprises the FR-targeting agent. Also disclosed are methods for use of such compositions for generating or enhancing an immune response to an antigen.

### II. Proteins

As used herein, a “protein” or “polypeptide” refers to a molecule comprising at least five amino acid residues. In some embodiments, proteins associated with (i.e., preferentially expressed in) first responder dendritic cells (also “FR-associated proteins”) are contemplated. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

As used herein, a “cell surface protein,” (also “surface protein” or “surface marker”) describes a protein which may be expressed on a surface (e.g., cell membrane) of a cell. A cell surface protein may be attached to a membrane of a cell. A cell surface protein may be embedded in a membrane of a cell. A cell surface protein may comprise one or more transmembrane regions. In some embodiments, cell surface proteins associated with (i.e. preferentially expressed in) first responder dendritic cells are contemplated.

A protein of the disclosure may be targeted, e.g., via an antibody or antibody fragment, a peptide configured to bind to the protein, or any other molecule capable of specifically binding to the protein. For example, an FR-associated protein may be targeted using an antibody for delivery of an antigen to FRs expressing the protein.

Examples of proteins which may be targeted using methods and compositions of the present disclosure include those provided in Table 1. In some embodiments, at least or at most 1, 2, 3, 4, 5, 6, 7, 8, 9, 10, 11, 12, 13, 14, 15, 16, 17, 18, 19, or 20 proteins of Table 1 are targeted, or more. Any one or more of the proteins of Table 1 may be excluded from embodiments of the disclosure. In some embodiments, proteins targeted using compositions and methods of the present disclosure include one or more of proteoglycan 2 (PRG2; also “Bone marrow proteoglycan” or “BMPG”), DNAX-activation protein 12 (DAP12; also “TYRO protein tyrosine kinase-binding protein” or “TYOBP”), Transmembrane protein 176A (TMEM176A; also “Hepatocellular carcinoma-associated antigen 112” or “HCA112”), Triggering receptor expressed on myeloid cells 2 (TREM2; also “TREM-2”), C-type lectin domain family 5 member A (CLC5A; also “CLEC5A”), CD206 (also “mannose receptor” or “MMR” or “MR”), and C9orfl35 (also “CFAP95” or “Protein CFAP95” or “Cilia- and flagella-associated protein 95”). Any one or more of these proteins may be excluded from embodiments of the disclosure.

### II. Adjuvants

Aspects of the present disclosure include adjuvants and methods for administering adjuvants to a subject. The immunogenicity of a particular composition can be enhanced by the use of non-specific stimulators of the immune response, known as adjuvants. Adjuvants that may be used in accordance with embodiments include, but are not limited to, IL-1, IL-2, IL-4, IL-7, IL-12, 7-interferon, GM-CSF, BCG, aluminum hydroxide, MDP compounds, such as thur-MDP and nor-MDP, CGP (MTP-PE), lipid A, and monophosphoryl lipid A (MPL). Other example adjuvants may include complete Freund's adjuvant (a non-specific stimulator of the immune response containing killed Mycobacterium tuberculosis), incomplete Freund's adjuvants, and/or aluminum hydroxide adjuvant.

In some embodiments, an adjuvant of the disclosure is a TLR agonist. A TLR agonist may be any molecule that, directly or indirectly, activates a TLR and/or stimulates TLR signaling. In some cases, a TLR agonist is a molecule that binds directly to a TLR. TLR agonists may be formulated into nanoparticles. Use of TLR agonists as adjuvants is described in, for example, Li et al., TLR Agonists as Adjuvants for Cancer Vaccines. Adv Exp Med Biol. 2017; 1024:195-212 (incorporated herein by reference in its entirety).

In some embodiments, the TLR agonist is one known in the art and/or described herein. The TLR agonists may include an agonist to TLR1 (e.g., peptidoglycan or triacyl lipoproteins), TLR2 (e.g., lipoteichoic acid; peptidoglycan from Bacillus subtilis, E. coli 0111:B4, Escherichia coli K12, or Staphylococcus aureus; atypical lipopolysaccharide (LPS) such as Leptospirosis LPS and Porphyromonas gingivalis LPS; a synthetic diacylated lipoprotein such as FSL-1 or Pam₂CSK₄; lipoarabinomannan or lipomannan from M. smegmatis; triacylated lipoproteins such as Pam₃CSK₄; lipoproteins such as MALP-2 and MALP-404 from mycoplasma; Borrelia burgdorferi OspA; Porin from Neisseria meningitidis or Haemophilus influenza; Propionibacterium acnes antigen mixtures; Yersinia LcrV; lipomannan from Mycobacterium or Mycobacterium tuberculosis; Trypanosoma cruzi GPI anchor; Schistosoma mansoni lysophosphatidylserine; Leishmania major lipophosphoglycan (LPG); Plasmodium falciparum glycophosphatidylinositol (GPI); zymosan; antigen mixtures from Aspergillus fumigatus or Candida albicans; and measles hemagglutinin), TLR3 (e.g., double-stranded RNA, polyadenylic-polyuridylic acid (Poly(A:U)); polyinosine-polycytidylic acid (Poly(I:C)); polyinosine-polycytidylic acid high molecular weight (Poly(I:C) HMW); and polyinosine-polycytidylic acid low molecular weight (Poly(I:C) LMW)), TLR4 (e.g., LPS from Escherichia coli and Salmonella species); TLR5 (e.g., Flagellin from B. subtilis, P. aeruginosa, or S. typhimurium), TLR8 (e.g., single stranded RNAs such as ssRNA with 6UUAU repeats, RNA homopolymer (ssPolyU naked), HIV-1 LTR-derived ssRNA (ssRNA40), or ssRNA with 2 GUCCUUCAA repeats (ssRNA-DR)), TLR7 (e.g., imidazoquinoline compound imiquimod, Imiquimod VacciGrade™ Gardiquimod VacciGrade™, or Gardiquimod™; adenine analog CL264; base analog CL307; guanosine analog loxoribine; TLR7/8 (e.g., thiazoquinoline compound CL075; imidazoquinoline compound CL097, 2Bxy, R848, or R848 VacciGrade™) TLR9 (e.g., CpG oligodeoxynucleotides (ODNs)); and TLR11 (e.g., Toxoplasma gondii Profilin).

### IV. Therapeutic Methods

The compositions of the disclosure may be used for in vivo, in vitro, or ex vivo administration. The route of administration of the composition may be, for example, intracutaneous, subcutaneous, intravenous, local, topical, and intraperitoneal administrations.

### A. Cancer Therapy

In some embodiments, the method further comprises administering a cancer therapy to the patient. The cancer therapy may be chosen based on expression level measurements, alone or in combination with a clinical risk score calculated for the patient. In some embodiments, the cancer therapy comprises a local cancer therapy. In some embodiments, the cancer therapy excludes a systemic cancer therapy. In some embodiments, the cancer therapy excludes a local therapy. In some embodiments, the cancer therapy comprises a local cancer therapy without the administration of a system cancer therapy. In some embodiments, the cancer therapy comprises an immunotherapy, which may be an immune checkpoint therapy. Any of these cancer therapies may also be excluded. Combinations of these therapies may also be administered.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. In some embodiments, the cancer is recurrent cancer. In some embodiments, the cancer is Stage I cancer. In some embodiments, the cancer is Stage II cancer. In some embodiments, the cancer is Stage III cancer. In some embodiments, the cancer is Stage IV cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the cancer is a recurrent cancer. In some embodiments, the cancer is Stage I cancer. In some embodiments, the cancer is Stage II cancer. In some embodiments, the cancer is Stage III cancer. In some embodiments, the cancer is Stage IV cancer.

It is contemplated that a cancer treatment may exclude any of the cancer treatments described herein. Furthermore, embodiments of the disclosure include patients that have been previously treated with a therapy described herein, are currently being treated with a therapy described herein, or have not been treated with a therapy described herein. In some embodiments, the patient is one that has been determined to be resistant to a therapy described herein. In some embodiments, the patient is one that has been determined to be sensitive to a therapy described herein.

### 1. Immunotherapy

In some embodiments, the methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immumotherapies are known in the art, and some are described below.

a. Activation of Co-Stimulatory Molecules

In some embodiments, the immunotherapy comprises an agonist of a co-stimulatory molecule. In some embodiments, the agonist comprises an agonist of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Agonists include agonistic antibodies, polypeptides, compounds, and nucleic acids.

b. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

c. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some embodiments, the CAR-T therapy targets CD19.

d. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

e. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death.

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein.

f. Checkpoint Inhibitors

Embodiments of the disclosure may include administration of immune checkpoint inhibitors, examples of which are further described below. As disclosed herein, “checkpoint inhibitor therapy” (also “immune checkpoint blockade therapy”, “immune checkpoint therapy”, “ICT,” “checkpoint blockade immunotherapy,” or “CBI”), refers to cancer therapy comprising providing one or more immune checkpoint inhibitors to a subject suffering from or suspected of having cancer.

### I. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDIO680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

### II. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO 01/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

### II. LAG3

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T cells, natural killer cells, B cells, and plasmacytoid dendritic cells. LAG3's main ligand is MHC class II, and it negatively regulates cellular proliferation, activation, and homeostasis of T cells, in a similar fashion to CTLA-4 and PD-1, and has been reported to play a role in Treg suppressive function. LAG3 also helps maintain CD8+ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

In some embodiments, the immune checkpoint inhibitor is an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or VH and/or VL domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, BI754111, AVA-017, or GSK2831781. The anti-LAG3 antibodies disclosed in: U.S. Pat. No. 9,505,839 (BMS-986016, also known as relatlimab); U.S. Pat. No. 10,711,060 (IMP-701, also known as LAG525); U.S. Pat. No. 9,244,059 (IMP731, also known as H5L7BW); U.S. Pat. No. 10,344,089 (25F7, also known as LAG3.1); WO 2016/028672 (MK-4280, also known as 28G-10); WO 2017/019894 (BAP050); Burova E., et al., J. ImmunoTherapy Cancer, 2016; 4 (Supp. 1):P195 (REGN3767); Yu, X., et al., mAbs, 2019; 11:6 (LBL-007) can be used in the methods disclosed herein. These and other anti-LAG-3 antibodies useful in the claimed invention can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

In some embodiments, the inhibitor comprises the heavy and light chain CDRs or VRs of an anti-LAG3 antibody. Accordingly, in one embodiment, the inhibitor comprises the CDR1, CDR2, and CDR3 domains of the VH region of an anti-LAG3 antibody, and the CDR1, CDR2 and CDR3 domains of the VL region of an anti-LAG3 antibody. In another embodiment, the antibody has at least about 70, 75, 80, 85, 90, 95, 97, or 99% (or any derivable range therein) variable region amino acid sequence identity with the above-mentioned antibodies.

### IV. TIM-3

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM_032782. TIM-3 is found on the surface IFNγ-producing CD4+ Th1 and CD8+ Tcl cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4+Th1-specific cell surface protein that regulates macrophage activation. Inhibitors of the disclosure may block one or more functions of TIM-3 activity.

In some embodiments, the immune checkpoint inhibitor is an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or VH and/or VL domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed invention can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to LAG3 also can be used.

In some embodiments, the inhibitor comprises the heavy and light chain CDRs or VRs of an anti-TIM-3 antibody. Accordingly, in one embodiment, the inhibitor comprises the CDR1, CDR2, and CDR3 domains of the VH region of an anti-TIM-3 antibody, and the CDR1, CDR2 and CDR3 domains of the VL region of an anti-TIM-3 antibody. In another embodiment, the antibody has at least about 70, 75, 80, 85, 90, 95, 97, or 99% (or any derivable range therein) variable region amino acid sequence identity with the above-mentioned antibodies.

### B. Autoimmune or Inflammatory Conditions

Aspects of the present disclosure comprise autoimmune or inflammatory conditions, and methods and compositions for treatment thereof. In some embodiments, the disclosed methods comprise treatment of an autoimmune condition using an FR-targeting agent and one or more therapeutic agents. Also disclosed are compositions comprising an FR-targeting agent and, in some cases, one or more therapeutic agents.

The autoimmune condition or inflammatory condition amenable for treatment may include, but not be limited to conditions such as diabetes (e.g. type 1 diabetes), graft rejection, arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and systemic juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), and adult onset diabetes mellitus (Type II diabetes) and autoimmune diabetes. Also contemplated are immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including polymyalgia rheumatica and gianT cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), Addison's disease, autoimmune neutropenia, pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, autoimmune or immune-mediated thrombocytopenia such as idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), experimental autoimmune encephalomyelitis, myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, gianT cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyl), and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, gianT cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, asperniogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, graft versus host disease, contact hypersensitivity, asthmatic airway hyperreaction, and endometriosis.

### V. Administration of Therapeutic Compositions

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### A. Pharmaceutical Formulations and Routes of Administration

Pharmaceutical compositions are provided herein that comprise an effective amount of one or more substances and/or additional agents dissolved or dispersed in a pharmaceutically acceptable carrier. The phrases “pharmaceutically acceptable” or “pharmacologically acceptable” refer to molecular entities and compositions that do not produce an adverse, allergic or other untoward reaction when administered to an animal, such as, for example, a human, as appropriate. The preparation of a pharmaceutical composition that contains at least one substance or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The compounds of the invention may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostatically, intrapleurally, intratracheally, intranasally, intravitreally, intravaginally, intrarectally, topically, intratumorally, intramuscularly, systemically, subcutaneously, subconjunctival, intravesicularly, mucosally, intrapericardially, intraumbilically, intraocularly, orally, locally, via inhalation (e.g., aerosol inhalation), via injection, via infusion, via continuous infusion, via localized perfusion bathing target cells directly, via a catheter, via a lavage, in cremes, in lipid compositions (e.g., liposomes), or by other method or any combination of the foregoing as would be known to one of ordinary skill in the art (see, for example, Remington's Pharmaceutical Sciences, 1990).

The actual dosage amount of a composition administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of a compound, polypeptide, antibody, or other molecule described herein. In other embodiments, the compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal, or combinations thereof.

The substance may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine, or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. It may be preferable to include isotonic agents, such as, for example, sugars, sodium chloride or combinations thereof.

In other embodiments, one may use eye drops, nasal solutions or sprays, aerosols or inhalants. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in certain embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the substance is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. In certain embodiments, carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar, or both.

Additional formulations which are suitable for other modes of administration include suppositories. Suppositories are solid dosage forms of various weights and shapes, usually medicated, for insertion into the rectum, vagina, or urethra. After insertion, suppositories soften, melt or dissolve in the cavity fluids. In general, for suppositories, traditional carriers may include, for example, polyalkylene glycols, triglycerides, or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, certain methods of preparation may include vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less than 0.5 ng/mg protein.

In particular embodiments, prolonged absorption of an injectable composition can be brought about by the use in the compositions of agents delaying absorption, such as, for example, aluminum monostearate, gelatin, or combinations thereof.

### VI. Kits

Certain aspects of the present invention also concern kits containing compositions of the invention or compositions to implement methods of the invention. In some embodiments, kits can be used to evaluate one or more biomarkers. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, there are kits for evaluating biomarker activity in a cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

In some aspects a kit of the present disclosure includes 1, 2, 3, 4, 5, or more FR-taargeting agents

In certain aspects, negative and/or positive control nucleic acids, probes, and inhibitors are included in some kit embodiments. In addition, a kit may include a sample that is a negative or positive control for methylation of one or more biomarkers.

Any embodiment of the disclosure involving specific biomarker by name is contemplated also to cover embodiments involving biomarkers whose sequences are at least 80, 81, 82, 83, 84, 85, 86, 87, 88, 89, 90, 91, 92, 93, 94, 95, 96, 97, 98, 99% identical to the mature sequence of the specified nucleic acid.

It is contemplated that any method or composition described herein can be implemented with respect to any other method or composition described herein and that different embodiments may be combined. The claims originally filed are contemplated to cover claims that are multiply dependent on any filed claim or combination of filed claims.

### EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1—Identification, Characterization, and Targeting of a Rare and Temporal Dendritic Cell State that Facilitates Adaptive Immune Responses

In this study, the inventors showed that a small population of DCs, disclosed herein as First Responder DCs (FRs; also “First Responders”), are necessary to simulate bulk innate cell TLR-mediated activation. The inventors identified and isolated these cells from heterogenous populations using fluorescently labeled TLR agonist conjugated microparticles (MPs). The inventors observed that a small percentage (set at 5%) accumulated a statistically unlikely percentage (>90%) of TLR bearing particles when the ratio of particles to cells was 1:1. As these cells were highly fluorescent, the inventors isolated FRs, observed unique transcriptional profiles, identified surface markers, and ultimately identified these cells as a subset of cDC2 cells. The inventors conducted experiments showing that the FR phenotype appears to be a transient state that APCs pass through on a 1 hr time-scale rather than a distinct cell subset. The inventors tested whether these cells alone were sufficient to mediate the response of the remaining APCs in a culture—concluding that they could trigger neighboring APCs via paracrine signaling. The inventors examined this further and found that for an adoptive transfer experiment of DCs, isolating only the 5% of FR cells and transferring them resulted in nearly the same level of adaptive immunity as transfer of the whole culture. This study shows how the transient state of FR is an important player in innate immune activation and have potential for therapeutic targeting.

### Results

### TLR Agonist Conjugated MP For Isolation of FRs

The primary hypothesis of these studies was that there exists a rare and toll like receptor (TLR) responsive dendritic cell (DC) state, disclosed herein as first responders (FRs; also “first responder DCs” or “first responder dendritic cells”). These cells assist and amplify innate activation and adaptive responses via paracrine signaling. The first responder state is characterized by (1) rapid accumulation of micron sized TLR agonist coated particles far exceeding statistical probability, (2) rapid, high-level expression of inflammatory cytokines which activate bystander cells, (3) a preliminarily identified set of cell surface markers which identify the transient cell state in naïve populations.

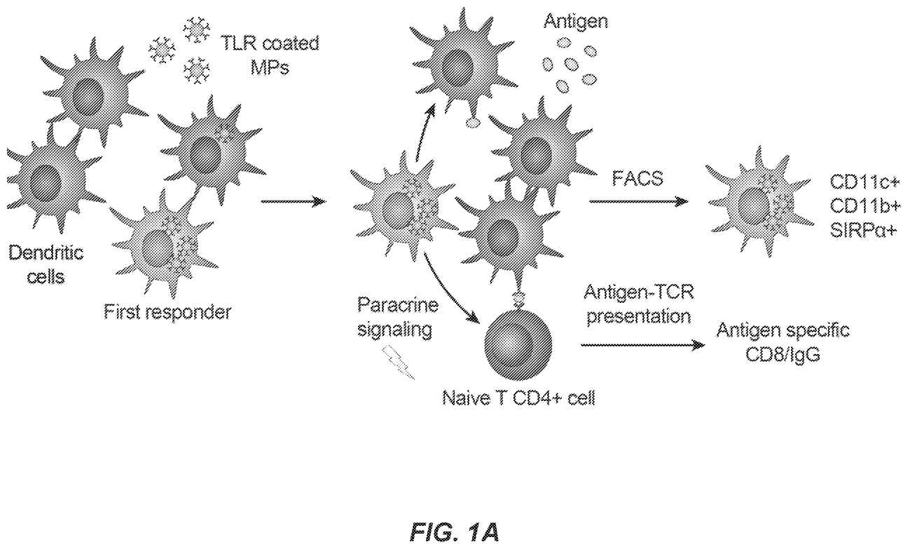

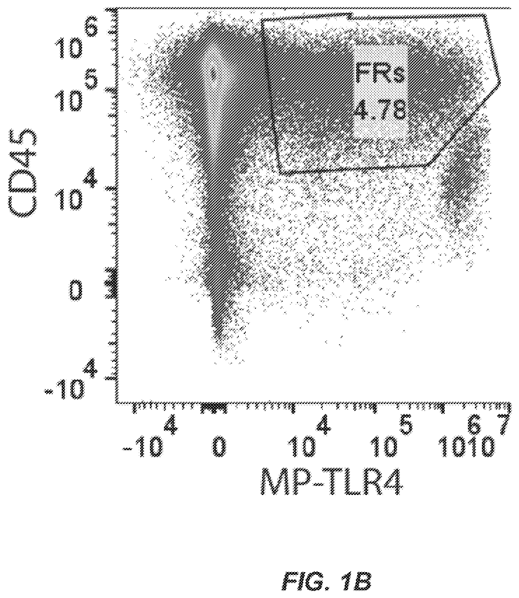

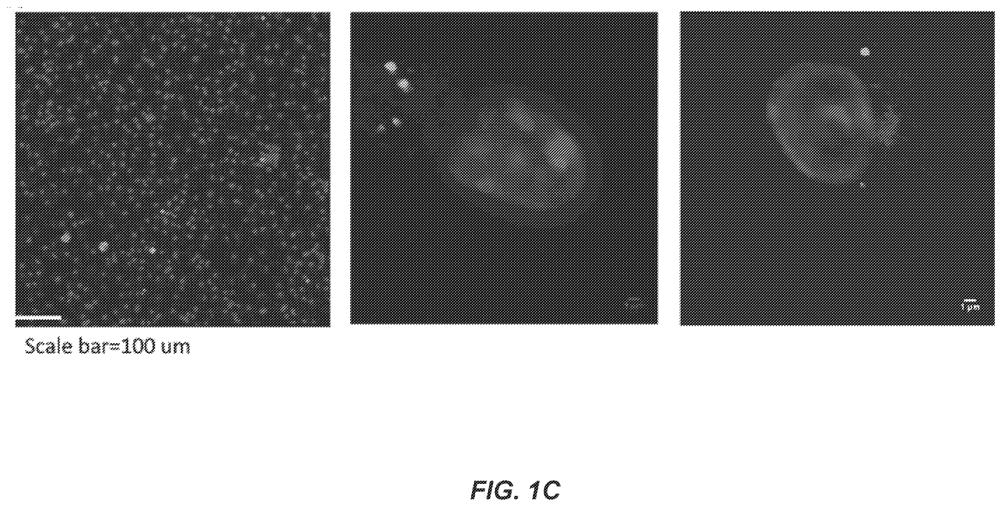

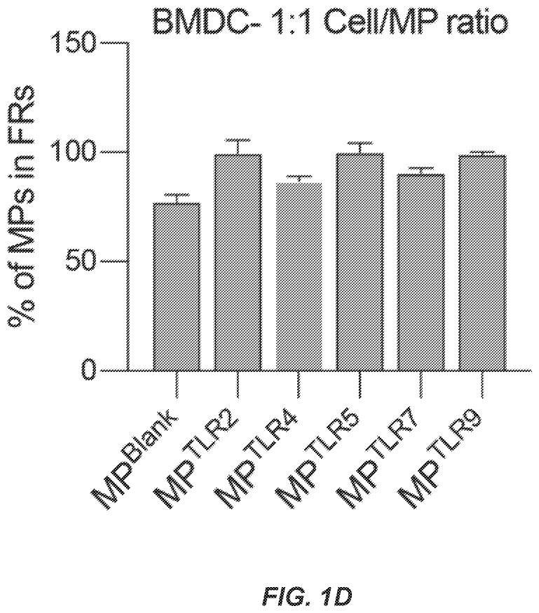

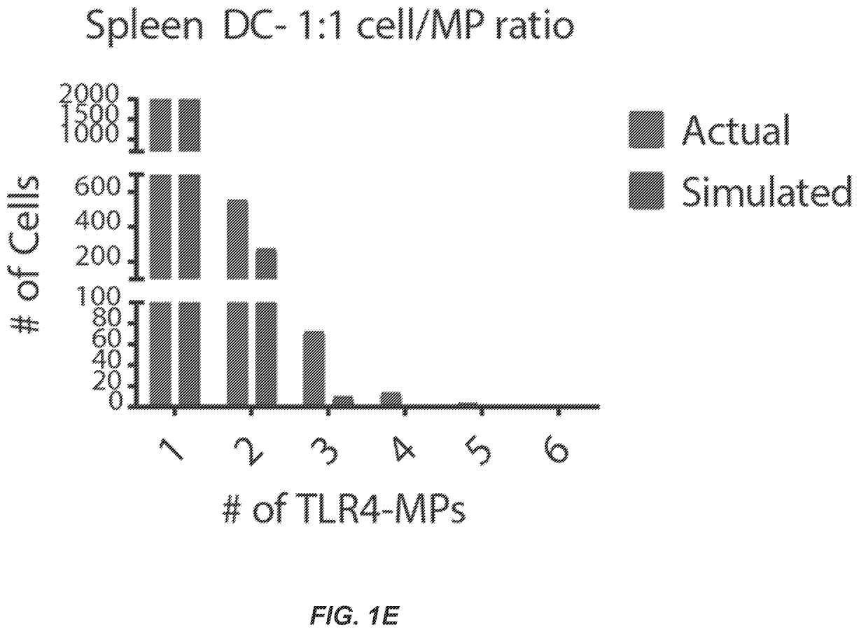

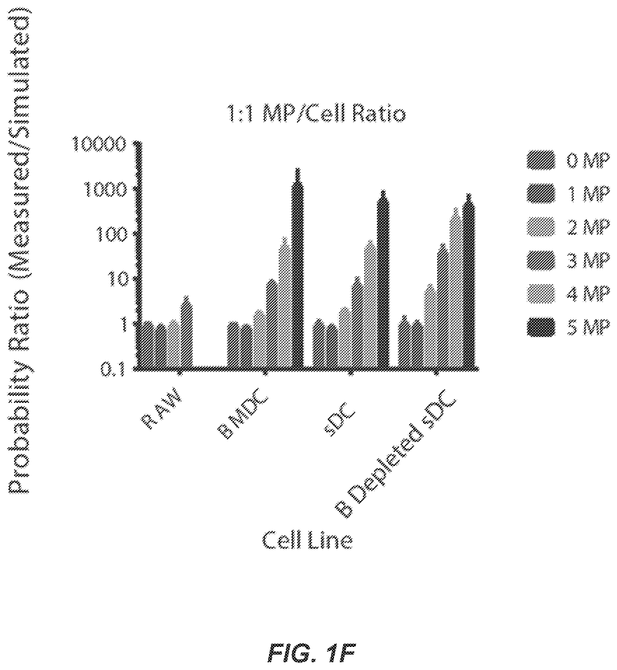

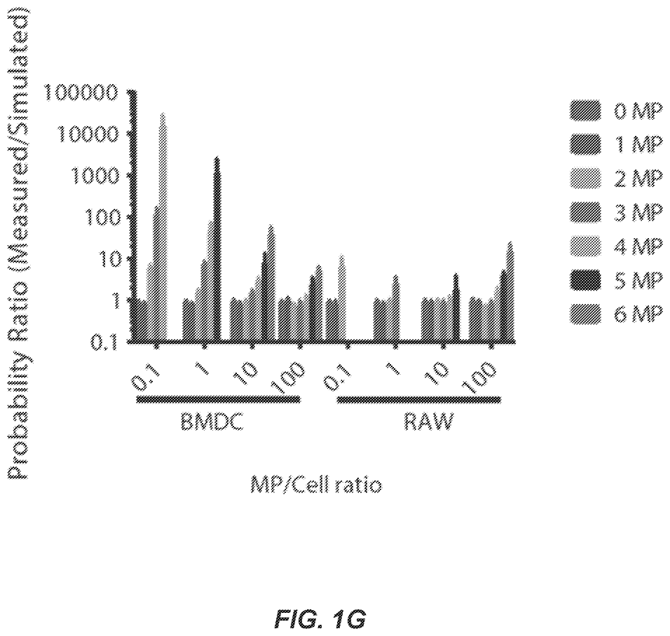

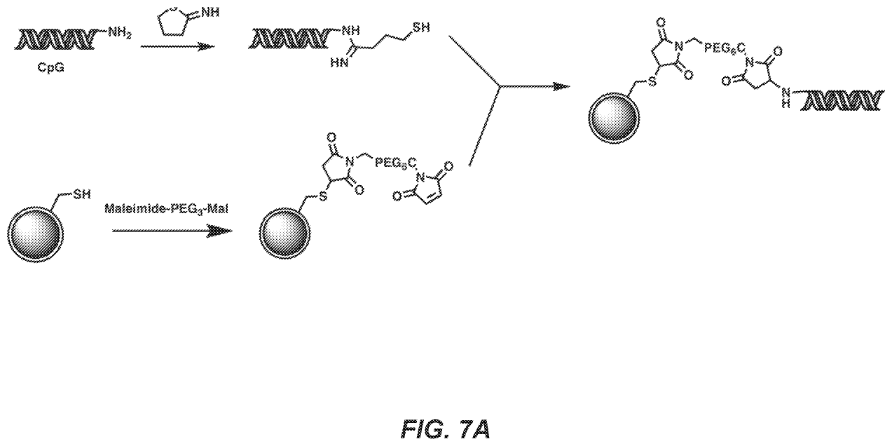

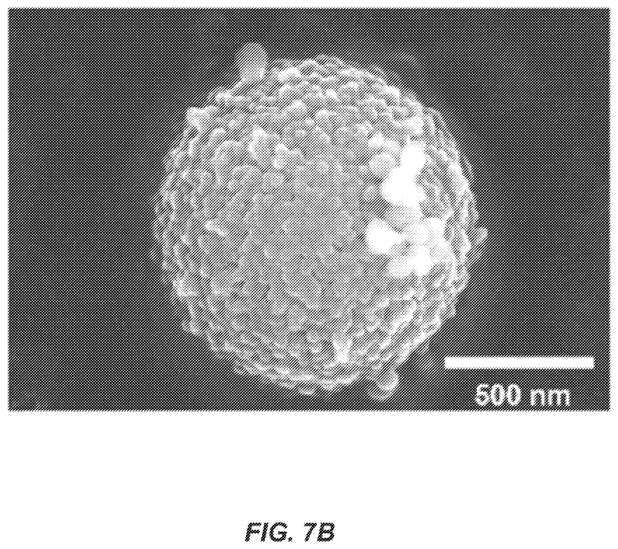

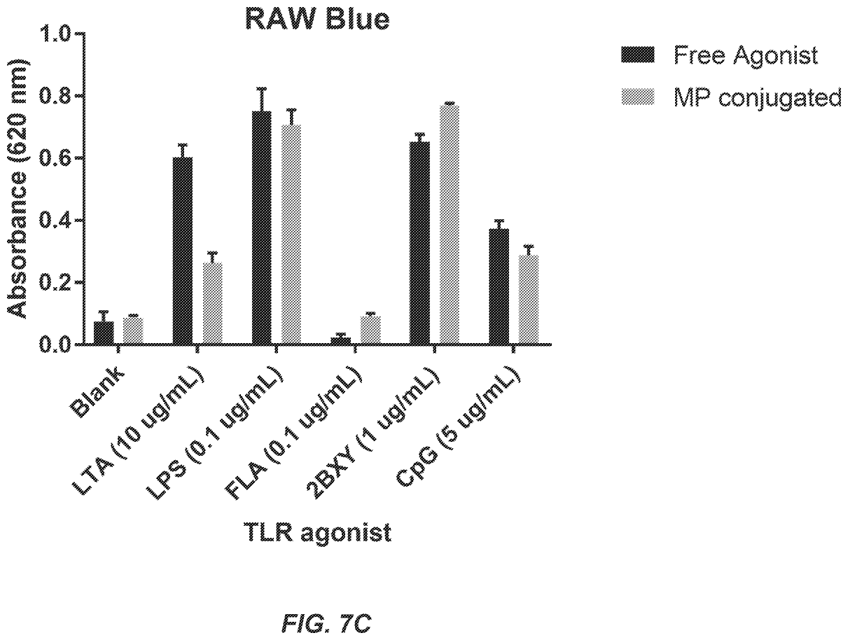

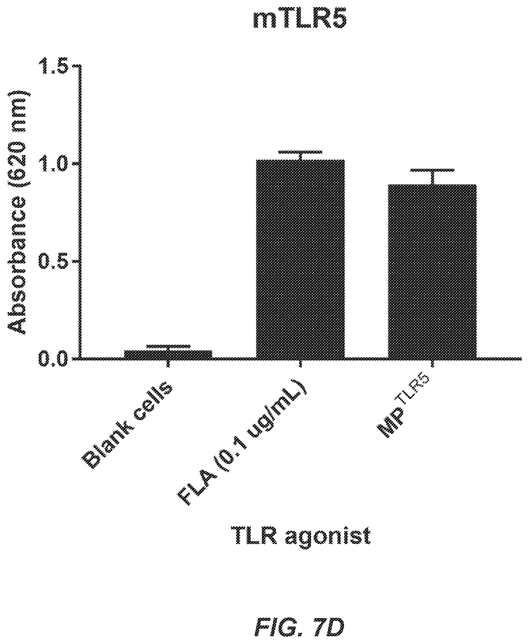

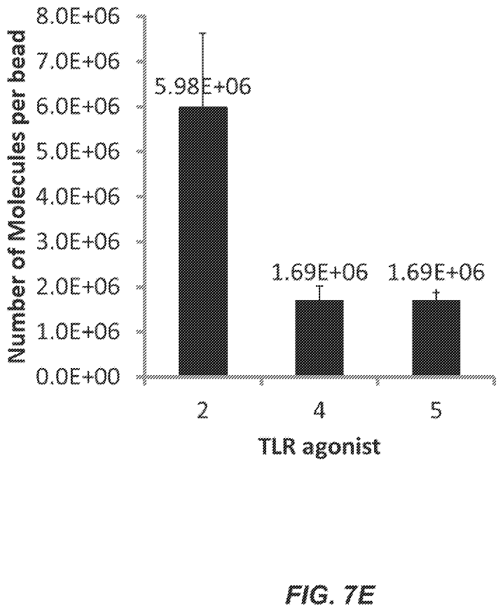

The first objective of this study was to develop a method to reliably isolate FRs from a heterogenous population of DCs. As FRs respond to TLR signaling², the inventors utilized a TLR conjugated micro-particles (MP) system to isolate FRs from other DCs, which the inventors co-opted from a previous study.³ The inventors synthesized 2 μm diameter FITC labeled polystyrene MPs and then coated them with a silicon-siloxane coating to reduce non-specific immune reactivity (FIGS. 7A-7E). The MPs were surface conjugated with one of 5 different TLR agonists (for TLR2, 4, 5, 7 and 9) using maleimide-thiol chemistry (FIGS. 7A-7E). Using previous established methods, the inventors calculated an approximation of the number of TLR agonists per MP using a BCA (bicinchoninic acid assay, quantifying the number of amide bonds) for the three agonists with amide bonds (LTA, LPS and FLA) and calculated approximately 1-10 million TLR agonists per MP (FIG. 7E). The inventors then incubated the MPs with innate immune cells and used particle uptake to isolate FRs from non-FRs (nFRs) (FIG. 1A).¹²

### FRs Uptake Significantly Greater Number of MP

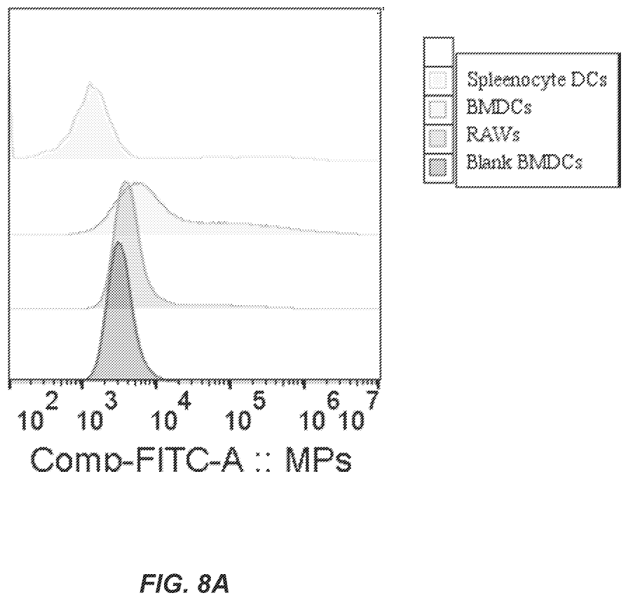

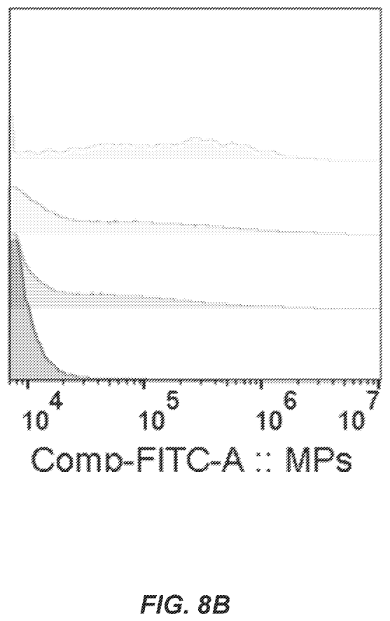

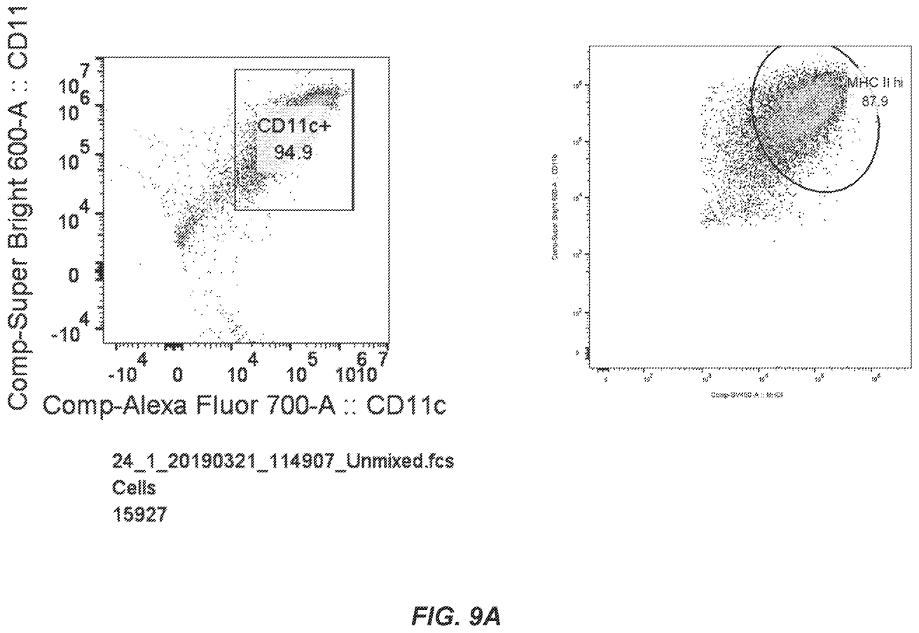

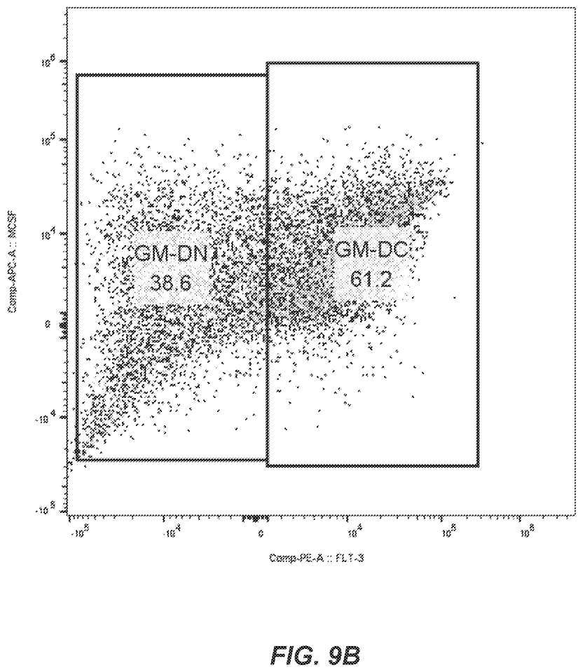

The inventors' initial observations of FRs were somewhat coincidental. In previous work, the inventors were calculating the number of agonists on a particle which activate a cell.¹³ During the course of these experiments, the inventors noticed a very large statistical anomaly
a small percentage of DCs had a disproportionally high MP signal that deviated greatly from a normal (Poisson) distribution. The inventors expected any immune cell would phagocytose MPs and especially MPs conjugated with TLR agonists, but based on a random distribution, it is expected to be a normal distribution of cells per MP assuming all DCs are homogeneous in their MP uptake properties.¹⁴ However, when the inventors incubated a heterogeneous cell line of either bone marrow derived dendritic cells (BMDCs) or spleen derived DCs (sDC) or a homogeneous control macrophage cell line (RAW) with MPs, the inventors observed a skew in higher fluorescence intensity for the heterogeneous cell lines but not for RAW cells (FIG. 1B, FIGS. 8A-8B, FIGS. 9A-9B for characterization of BMDCs). This effect was also seen via confocal microscopy, where MPs aggregate mainly in a select few BMDCs. At the same time, most of the TNF-α secretion was localized to the same few cells (FIG. 1C). To further quantify this shift, the inventors used Image Stream flow cytometry (ISX) to count the number of particles phagocytosed per cell. The inventors observed that >90% of all MPs (an LPS conjugated MP) were phagocytosed by <5% of all BMDCs (FIG. 1D). This cutoff of top 5% of FITC signal was used to define FRs in all following experiments in this study.

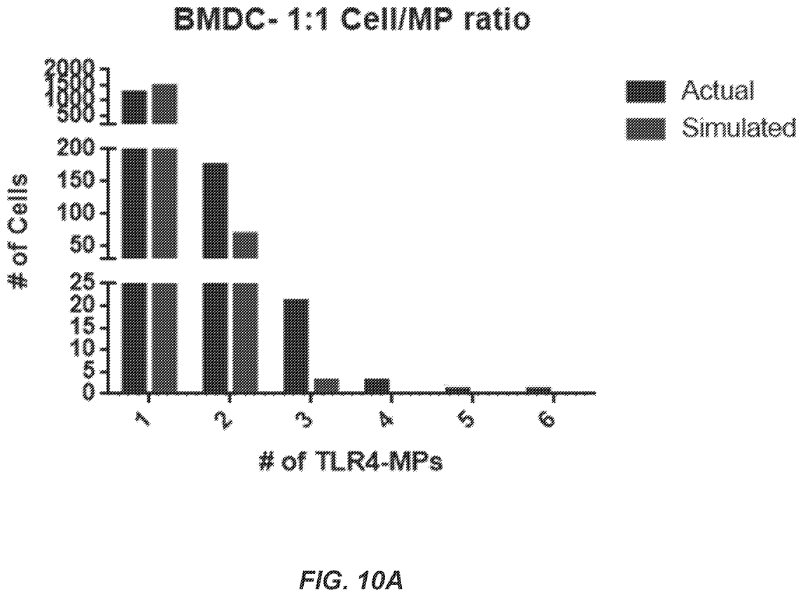

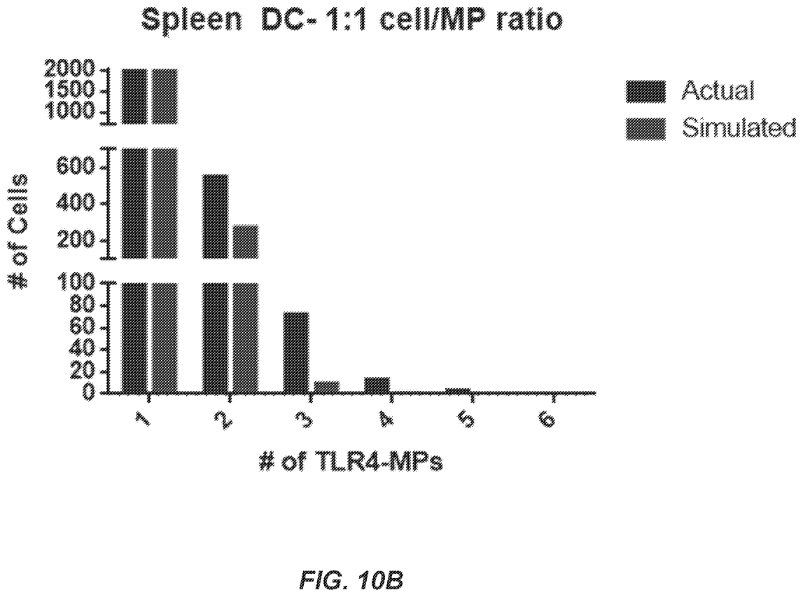

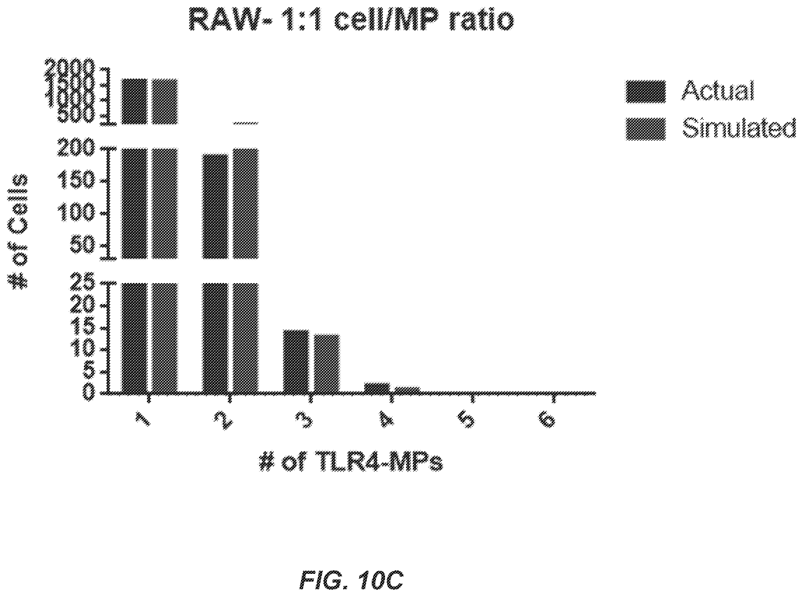

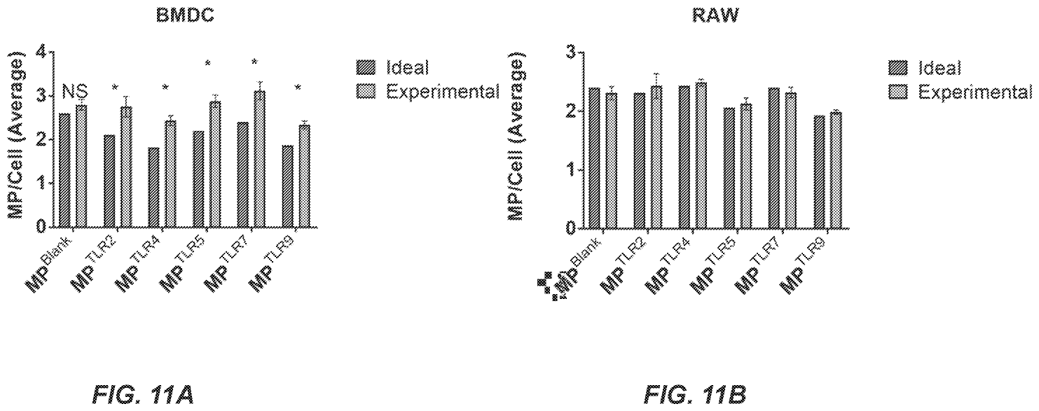

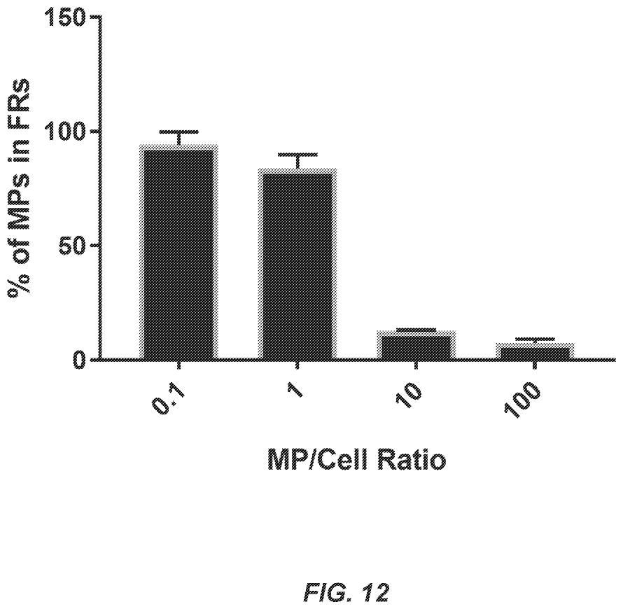

To explore this phenomenon further, the inventors analyzed sDC or BMDCs via ISX and observed an unusually high number of cells with >2 MPs per cell. When compared to a standard “random” Poisson distribution, there was a larger number of cells with >2 MP for sDCs and BMDCs but not for the RAW control cell line (FIG. 1E, FIGS. 10A-10C). The inventors were intrigued by this dramatic shift for a small number of MPs in the heterogeneous cell lines and hypothesized if this increase could be explained by normal statistical variance or if there was something distinctive about this sub-population of cells. To do this, the inventors further compared the standard Poisson distribution to the inventors' measured result, normalizing both the number of cells analyzed and the total number of MPs uptaken for each experimental condition and calculated a ratio of measured probability a cell has uptaken a certain number of MPs over the “simulated” probability based on the Poission distribution (FIG. 1F). For heterogeneous cell mixtures (BMDCs, sDCs or sDCs depleted of B cells), there is an increase in the probability ratio for cells with >2 MPs, but not for RAW cells (FIG. 1F). This result was also observed in all other MP formulations (FIG. 11). This result is unexpected, as similar studies using TLR coated MPs and BMDCs did not observe high concentration of particles in certain cells.¹⁵ The inventors believe this to be an effect of MP to cell ratio during incubation, as previous studies used a MP to cell ratio of greater than 10, whereas the inventors' experiments used a lower 1:1 MP/cell ratio. The inventors compared four different MP to cell ratios in a similar experiment to that shown in FIG. 1F and determined that this increase in MP uptake for FRs is only seen in lower MP/cell ratios, while higher ratios (10, 100 to 1 MP to cell) had >15% of MPs contained in FRs (FIG. 1G, FIG. 12). It is likely that at higher ratios, MPs will “saturate” all cells and obscure the increased uptake of MPs in FRs, which might explain why the FR phenomena was not observed previously.

FRs are Primarily Central Dendritic Cells Type 2 (cDC2) and FRs are Expressed in Human Monocyte derived DCs (moDCs)

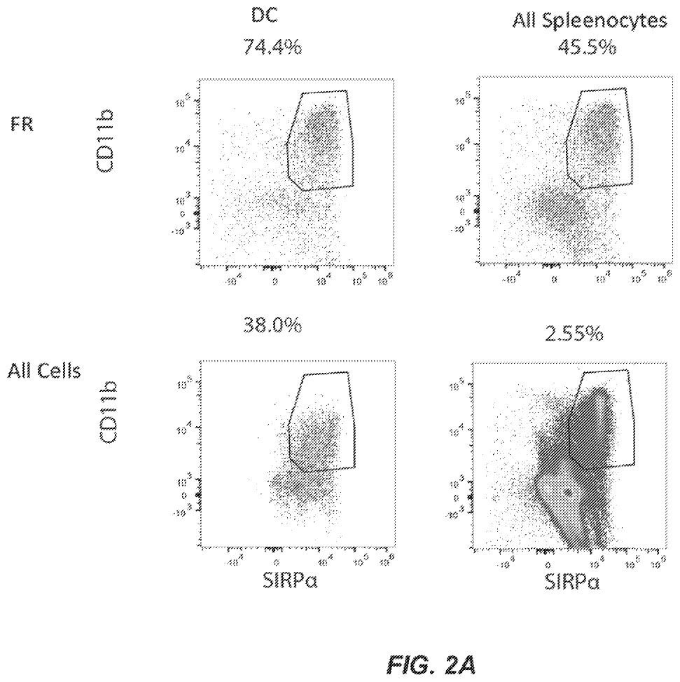

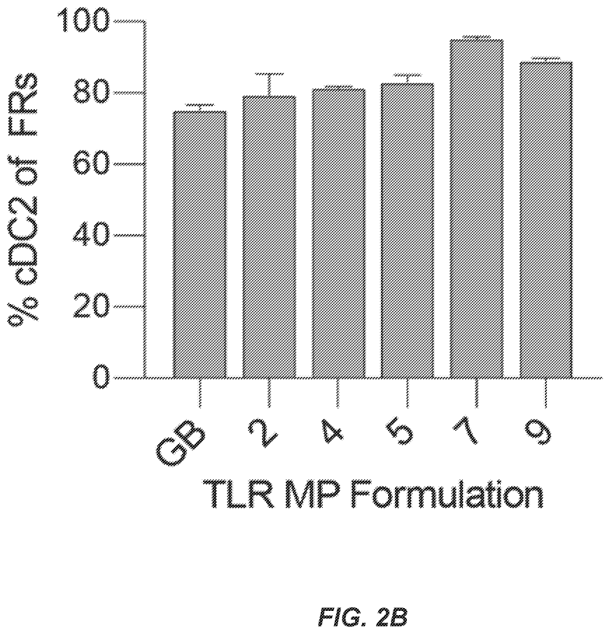

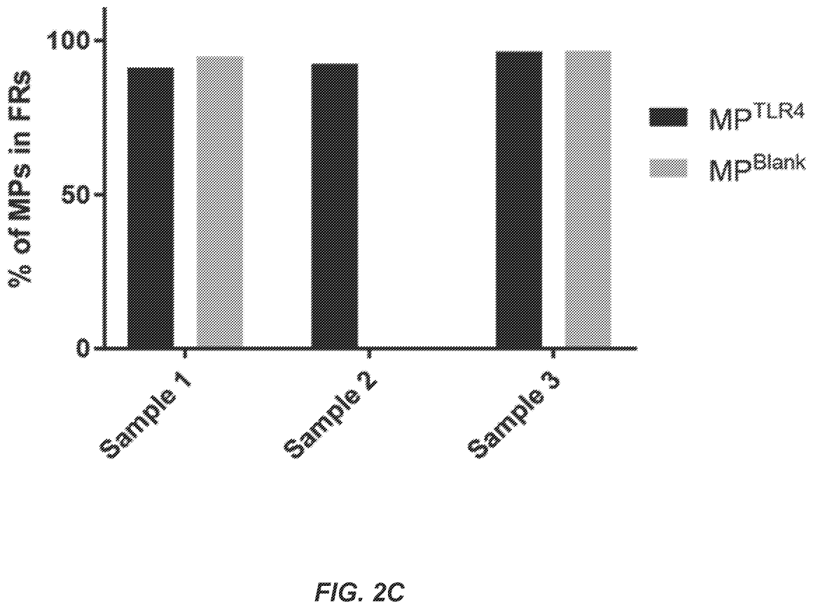

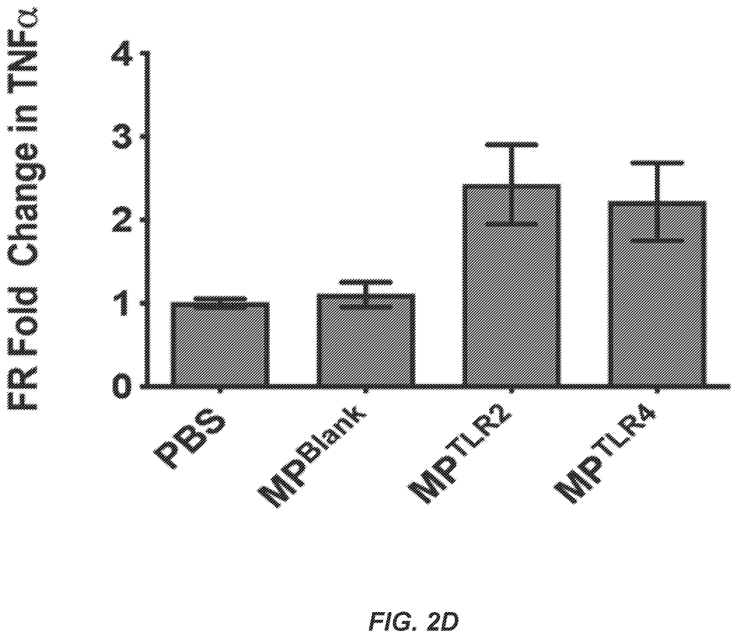

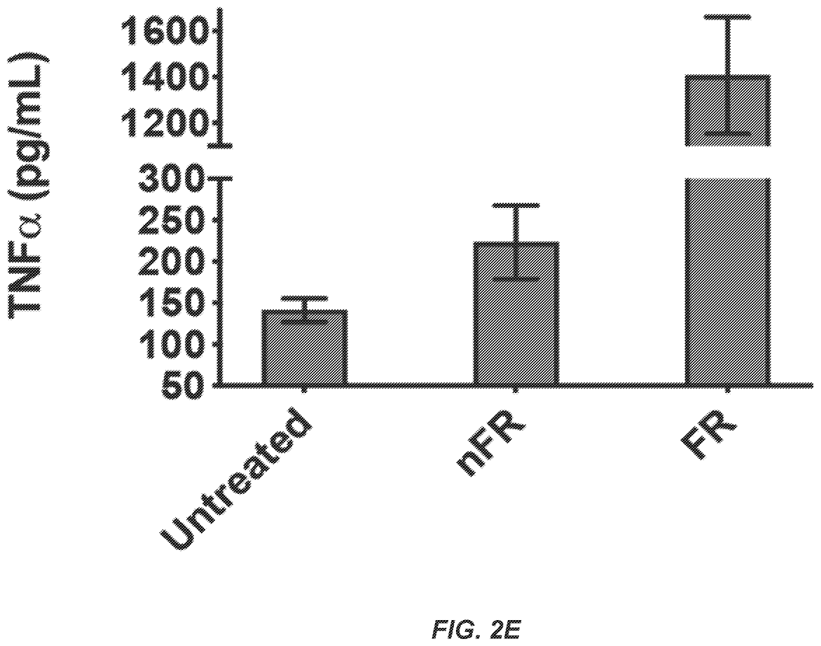

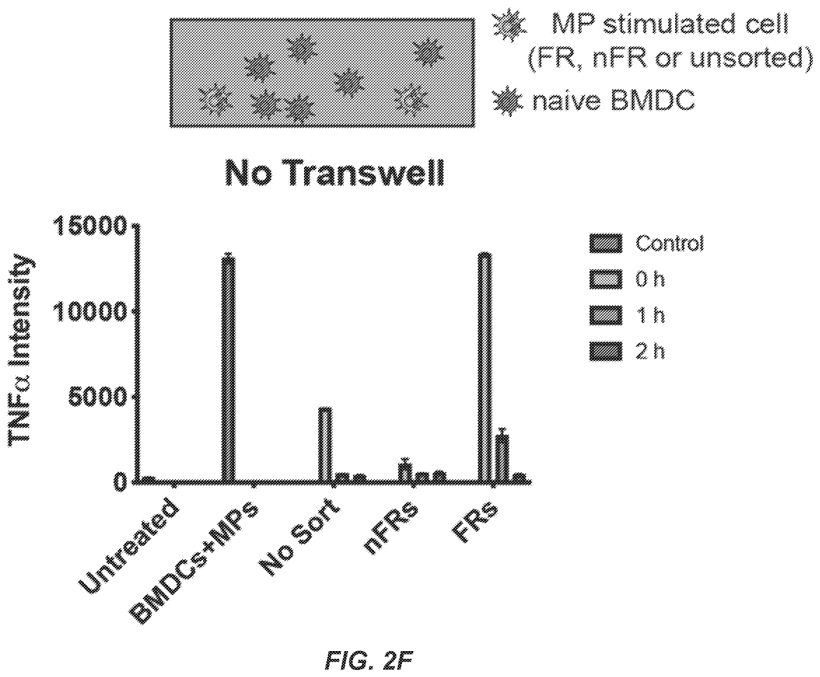

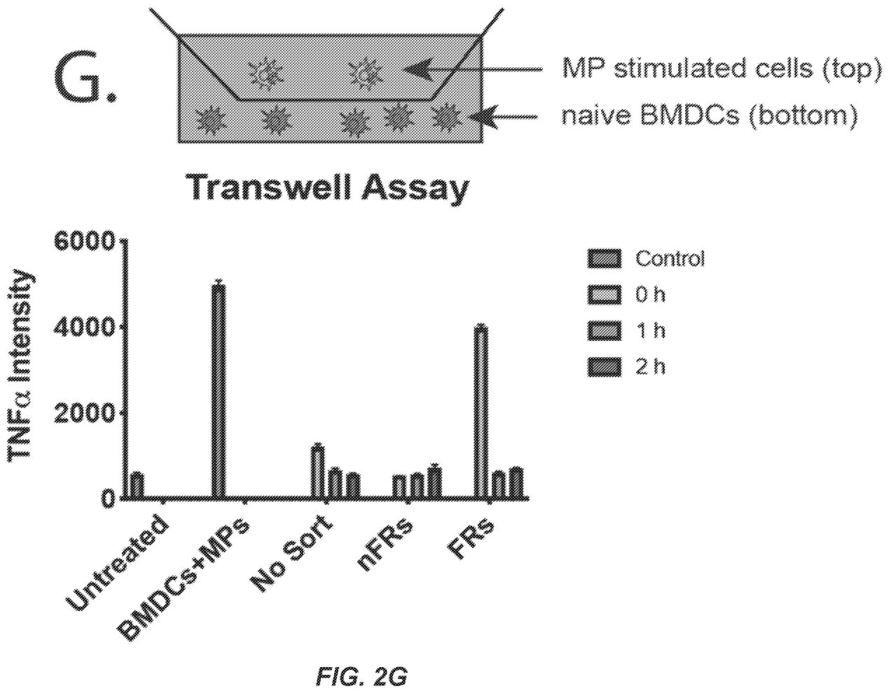

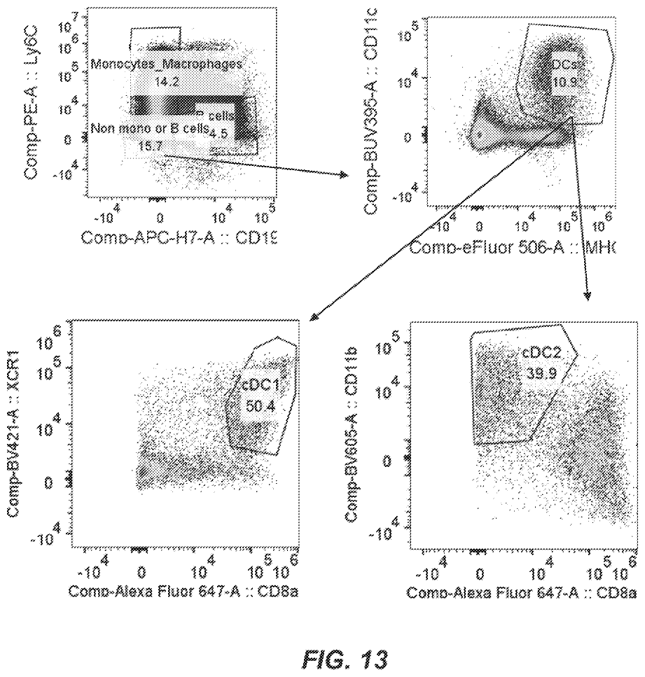

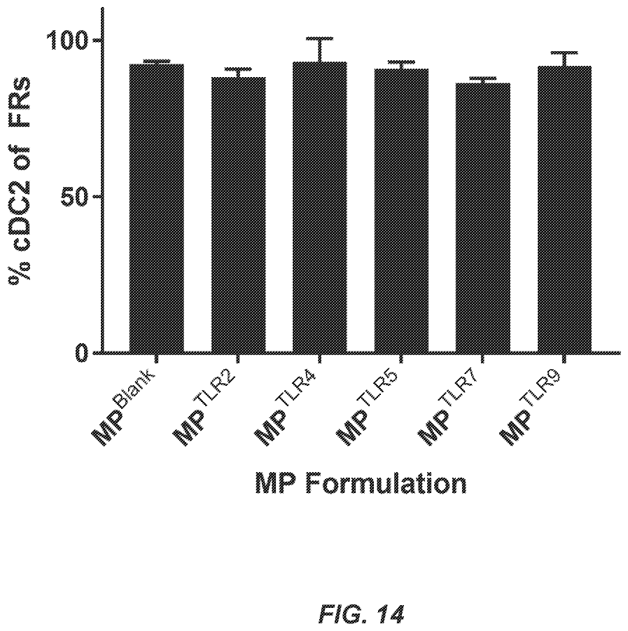

The inventors sought to phenotype FRs to determine if they are an already described phenotype of APCs. The inventors phenotyped spleenocytes incubated with MP^{TLR4} to determine if FRs match one of the major DC subsets, with a focus on cDC1 or cDC2 (see FIG. 13 for gating strategy). When isolating FRs, the inventors observed an increase in the number of cells in the cDC2 compartment (SIRPα+, CD1 lb+) for FRs compared to non-FRs (FIG. 2A). When gated on the DC compartment (CD11c+, MHCII hi), over 70% of FRs were also cDC2, while only 38% of DCs are naturally cDC2. The increase in cDC2 percentages were also seen when gated on all CD45+ cells and for BMDCs (FIG. 2A). The inventors observed a similar increase in cDC2 incidence for FR populations with spleenocytes incubated with other TLR agonist conjugated MPs, indicating that the FR state is primarily a part of the cDC2 populations (FIG. 2B) This same result in seen in resident skin DCs, where mouse footpads injected with MPs primarily are uptaken by resident cDC2 cells (FIG. 14).

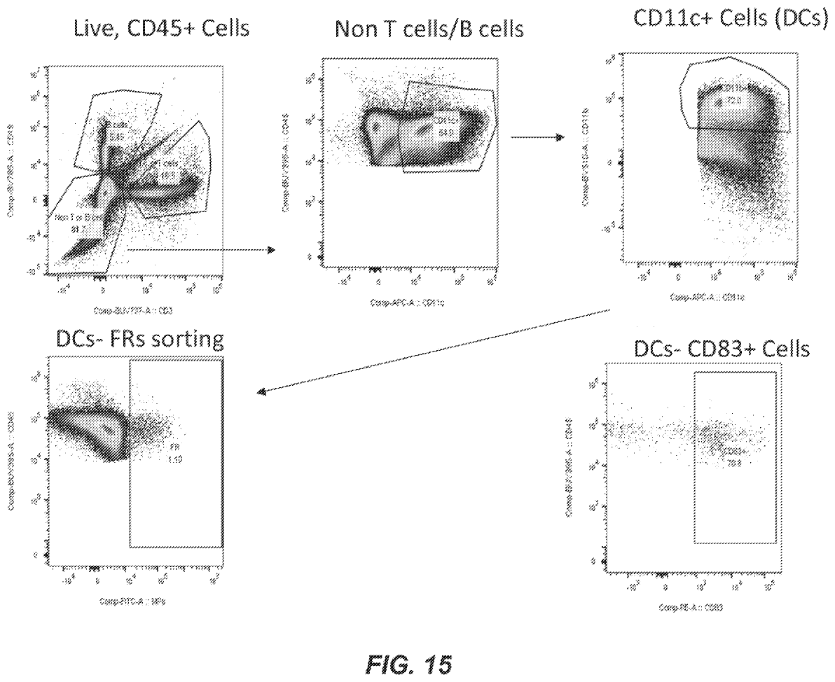

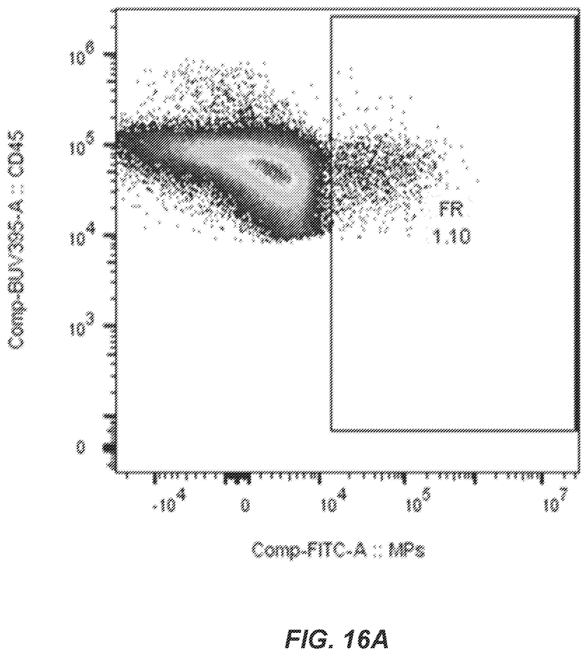

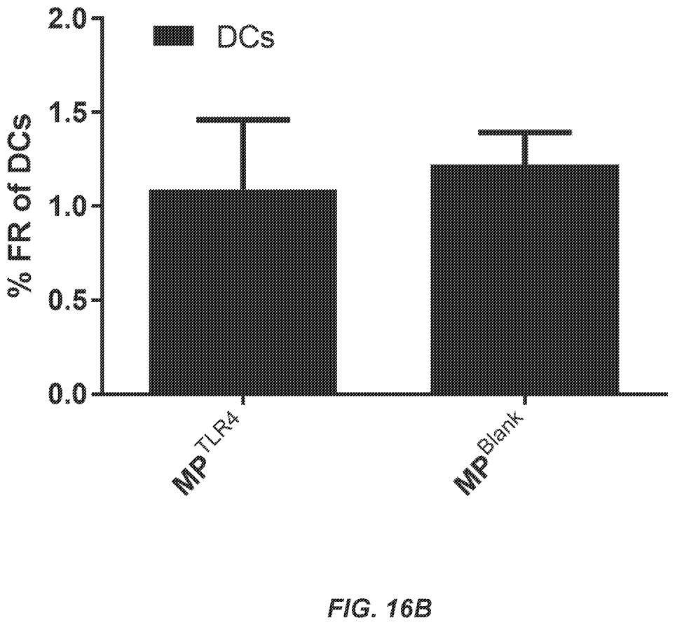

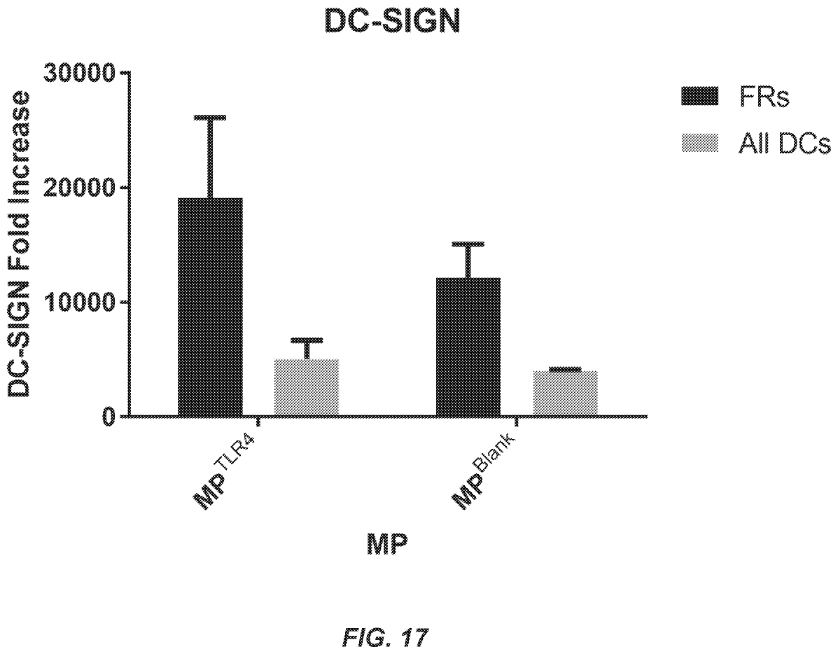

The inventors also observed FRs populations in human monocyte derived DCs. PBMCs (peripheral blood mononuclear cells) were purchased and derived into moDCs using IL-4 and GM-CSF treatment.¹⁶ The inventors observed that moDCs show similar patterns of MP uptake, but with the majority of MP signal in approximately 1% of DCs (CD11c+, CD45+ cells) phagocytosing over 90% of MPs (FIG. 2C, FIG. 15, FIGS. 16A-16B). The inventors also observed that human FRs overexpress the immaturity DC marker, DC-SIGN (FIG. 17).

FRs have Increased TLR Activation, Coordinate Local APC Activation Via Paracrine Signaling and are Temporally Controlled

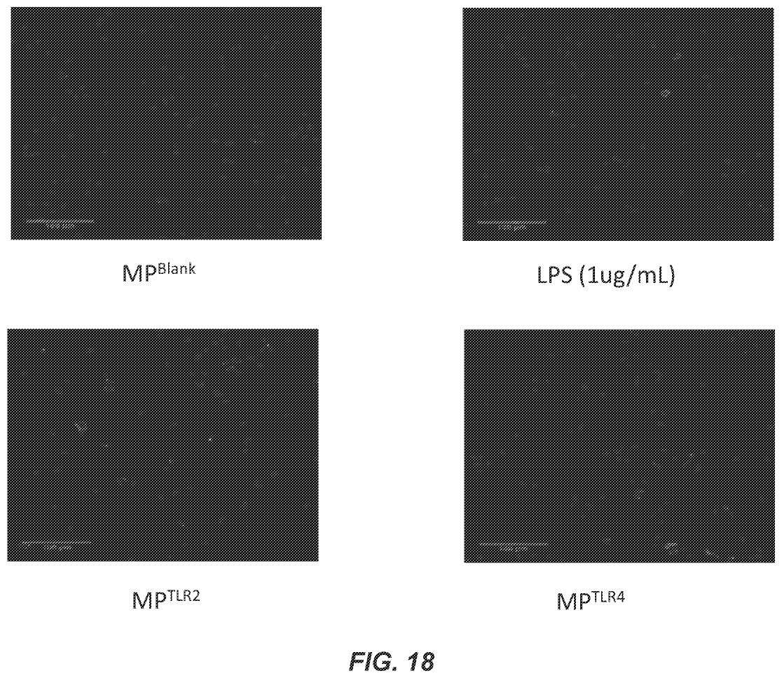

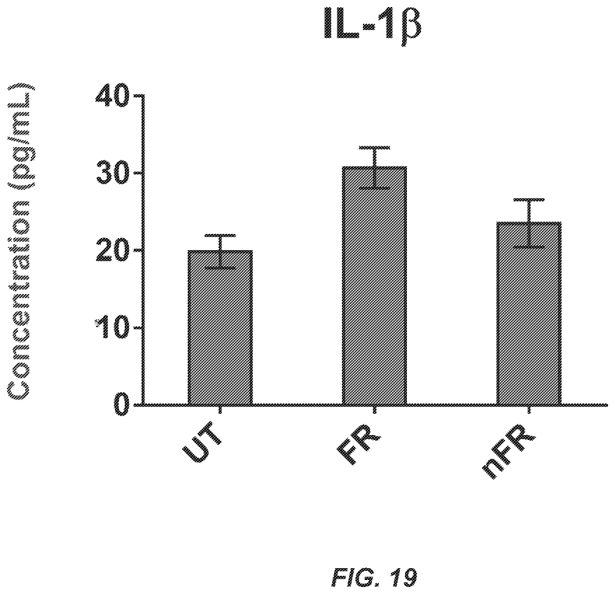

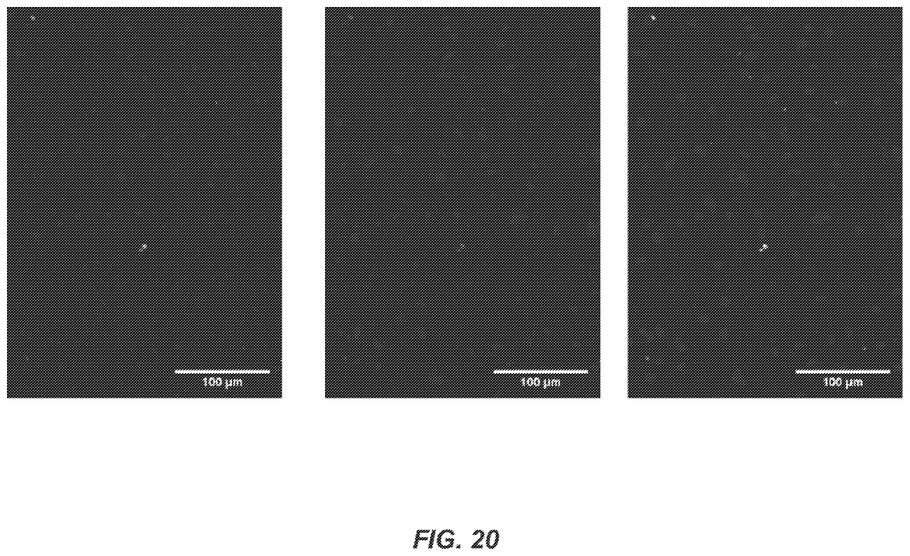

After phenotyping the FRs and determining that they are primarily part of the cDC2 population, the inventors were interested in studying their cytokine profiles and kinetics of activation. Since the FRs are the first cells to phagocytose MPs with PRRs, the inventors hypothesized that the FRs could also play a large role in initiating the innate immune response. As such, the inventors wanted to gain insight into the nature of their immune response. There were two major questions that the inventors wanted to answer: 1) How does the immune response of FRs differ from that of all the other cells which the inventors refer to as non-first responders (nFRs)? 2) What is the kinetics of the FR immune response and how do these kinetics influence bulk immune activation in vitro? The inventors sought to validate these questions in vitro and then to explore them in vivo. To probe the first question, the inventors investigated the cytokine expression of the FRs compared to the nFRs. Because the inventors had observed that FRs produce high levels of cytokines, the inventors limited intercellular cytokine signaling, by preincubated naïve BMDCs with Brefeldin A. Then the inventors stimulated 100 k BMDCs with MP^{TLR}-X (x=blank, 2, 4) in a 1:1 ratio for 1 hr, washed and incubated with Brefeldin A for 16 hrs and analyzed the cells with Imagestream flow cytometry (ISX) to determine TNFα expression. TNFα is a strong proinflammatory cytokine, and the FRs had a two-fold increase in TNFα relative to nFRs indicating that FRs produce higher levels of proinflammatory cytokines than the nFRs.¹⁷ (FIG. 2D). The inventors also observed this increase via confocal microscopy (FIG. 18). To further probe the cytokine profile of the FRs, the inventors looked at TNFα release in FRs and nFRs populations after stimulating naïve BMDCs with MP^{TLR-4} at 1:1 ratio. The FRs and nFRs were isolated via FACS, washed, and resuspended in media at a concentration of 1 million cells per mL. After 1 h, the supernatant was collected and the TNFα was measured via cytokine bead array (BD Biosciences). The supernatant was collected after 1 h and the inventors observed that a small percent of cells, the FRs, are responsible for significantly more TNFα secretion than the nFRs. in the nFR cell population the inventors observe that the TNFα secretion has relatively small increase (˜50%) relative to baseline. However, the FR population secretes significantly more TNFα than the nFR population. The measured TNFα levels secreted by the FRs is 1406 pg/mL, almost a 10-fold increase from the untreated population (FIG. 2E). This result indicates that the FRs, on average, secretes over 6 times the amount of TNFα per cell compared to the nFRs. From these data, the inventors hypothesized that the FRs could be key for stimulating immune activation in neighboring cells. The inventors also noted that IL-1β was not increased in FR populations or in any cell population, indicating that the inventors' MPs do not activate inflammasome activity (FIG. 19).¹⁵ The inventors further confirmed this lack of inflammasome activity by demonstrating that MPs do not escape lysosomes in FRs (FIG. 20).

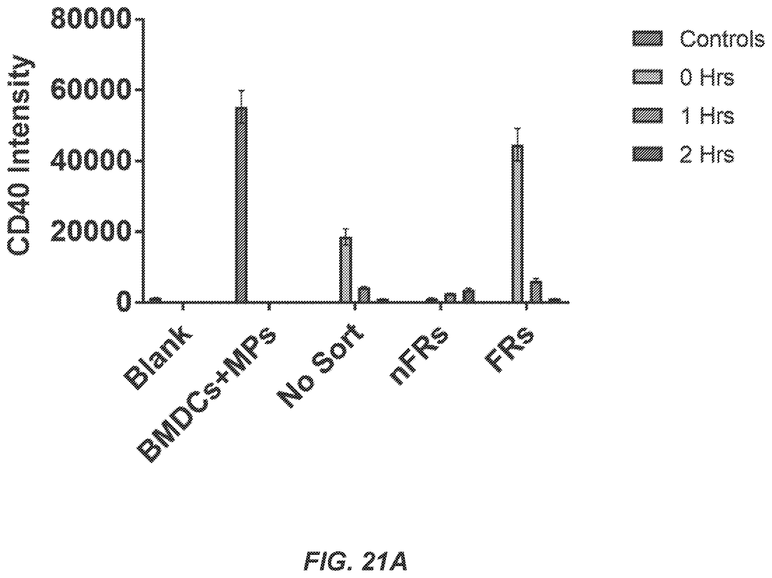

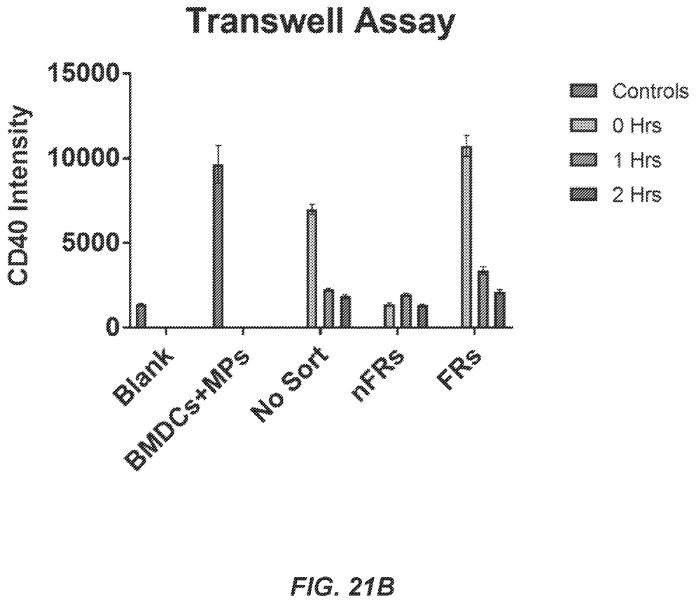

After observing the high TNFα levels produced by FRs, the inventors sought to determine if FRs can induce a pro-inflammatory response in an in vitro population of naïve cells and also the timeline of when FRs signal to neighboring cells. To test these questions, the inventors activated naïve BMDCs with M^{TLR4} and isolated the FRs and nFRs via FACS. The inventors isolated cells immediately after sorting—designated as t=0, 1, or 2 h after incubation. FRs (top 5% of MP signal), nFRs (bottom 90% of MP signal), or unsorted BMDCs were washed after sorting and then added back to a fresh-culture of 1 million naïve BMDCs from the same pool of cells in a 1:10 ratio. After 16 h, immune activation was determined by measuring the intensity of TNFα and CD40, a costimulatory molecule and marker of immune activation.¹⁸ The inventors only observed high activation in BMDCs that were subjected to FRs at 0 h. From this, the inventors concluded that the FRs are necessary for bulk in vitro activation of BMDCs. Interestingly, the inventors measured only background expression of CD40 and TNFα when the FRs were incubated for 1 or 2 h before addition to the naïve BMDCs, indicating that the FR response very short-lived. Furthermore, in the cultures stimulated with nFRs, only background levels of TNFα and CD40 were measured, indicating that nFRs are not sufficient to stimulate in vitro immune response (FIGS. 2F-2G, FIGS. 21A-B).

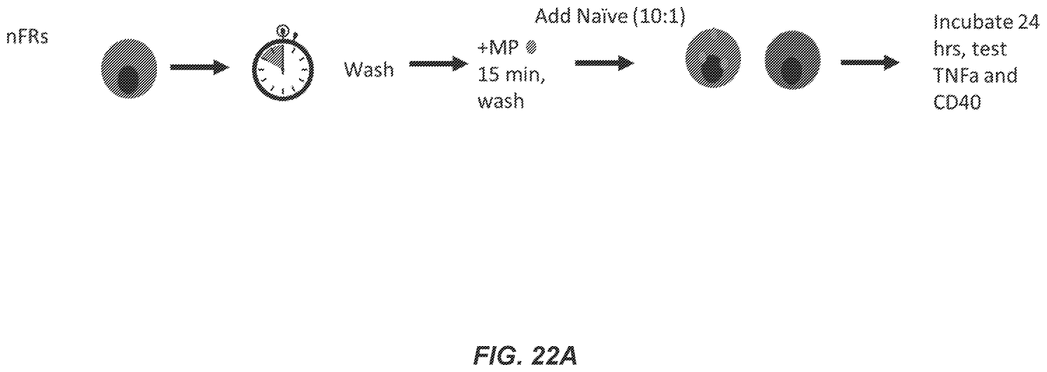

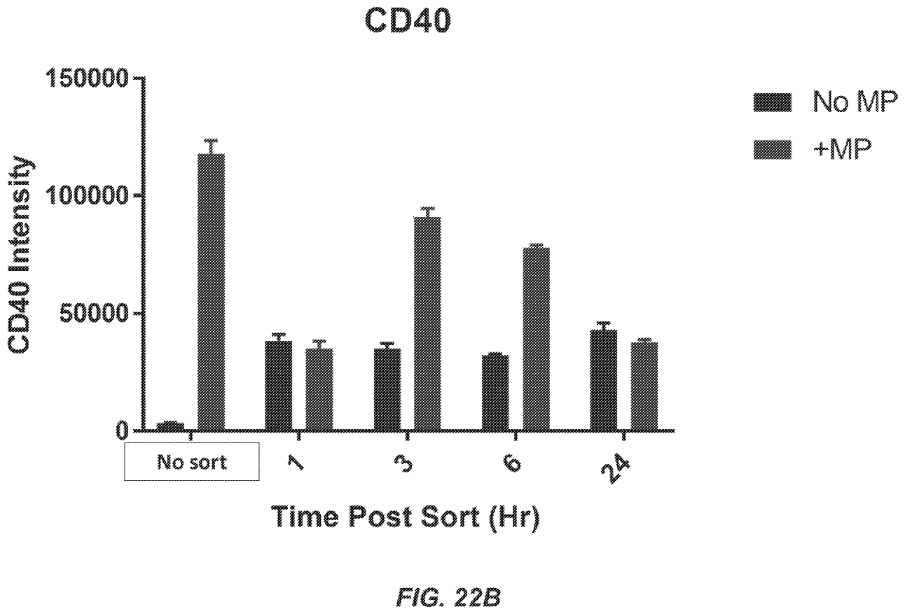

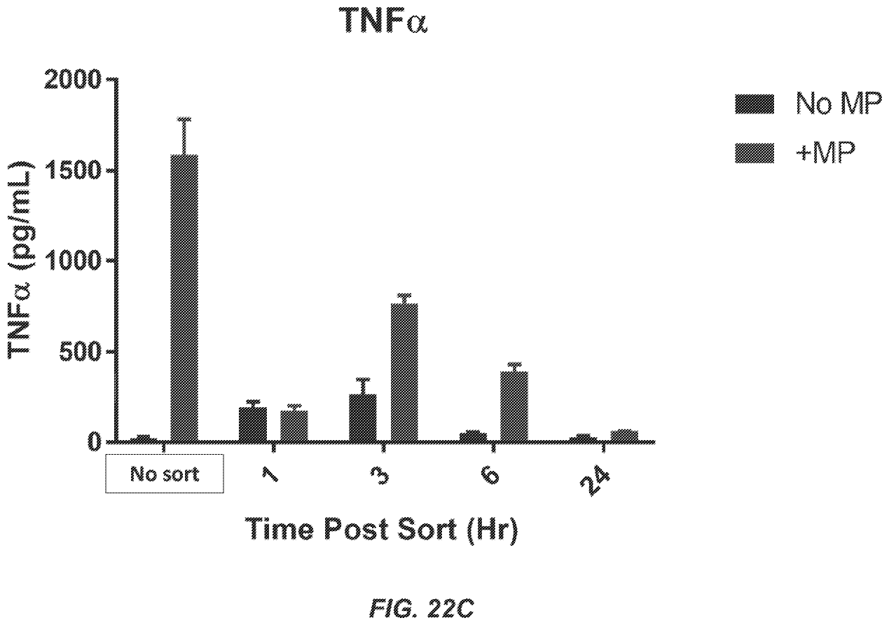

After determining that FRs are a critical component of obtaining an in vitro response, the inventors wanted to investigate whether the activation relied only on soluble factors. To test this, the inventors repeated this experiment using a transwell membrane where the FRs, nFRs, and unsorted BMDCs were plated on top of the membrane and the naïve cells were plated below the membrane. Similarly, the inventors observed higher levels of CD40 and TNFα at 0 h, only when FRs were added to naïve cells (FIG. 2F, FIGS. 21A-B). These data indicate that secreted proteins working via paracrine signaling, driven by FRs, play an important role in the bulk activation of BMDCs, but only on a short, <1 h timescale after initial FR stimulation. It is important to note that this activation only occurs when the FRs are immediately added to the naïve BMDCs. If the FRs sit for >1 or 2 h, the responses of the whole in vitro culture give responses similar to unstimulated cultures. Most intriguing, the inventors observed that the FR state is periodic and renewable. When the inventors isolated nFRs, upon further culture, these isolated cells would then product FR-like cells which could be used isolated, stimulated and restimulate a naïve culture of BMDCs in an identical manner to initial FRs. However, this phenomenon only occurs after nFR rest for several hours after isolating the first set of FR (FIGS. 22A-22C) indicating that the FR state renews on a multi-hour period. From these experiments, the inventors conclude that FRs are both sufficient and necessary to initiate immune activation in vitro in BMDCs but can only perform this bulk stimulation on a short timescale after their initial activation.

### FRs are Necessary and Sufficient for Generating Antigen Specific Responses Via BMDC Adoptive Transfer

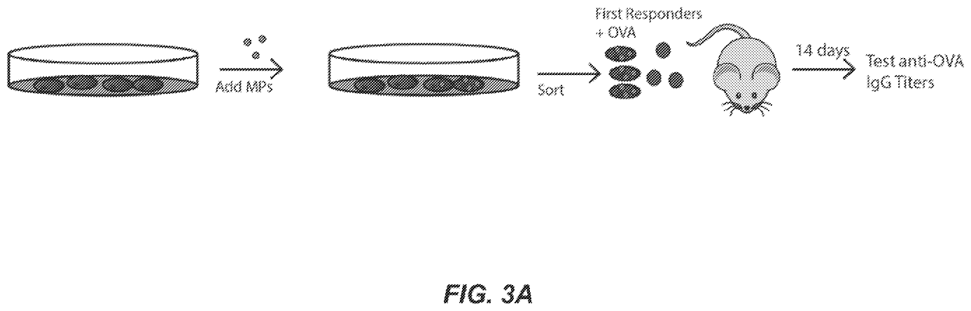

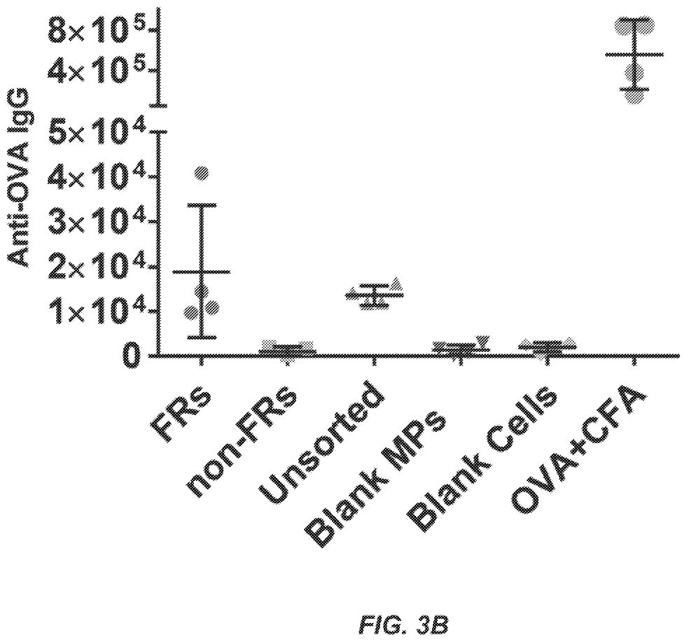

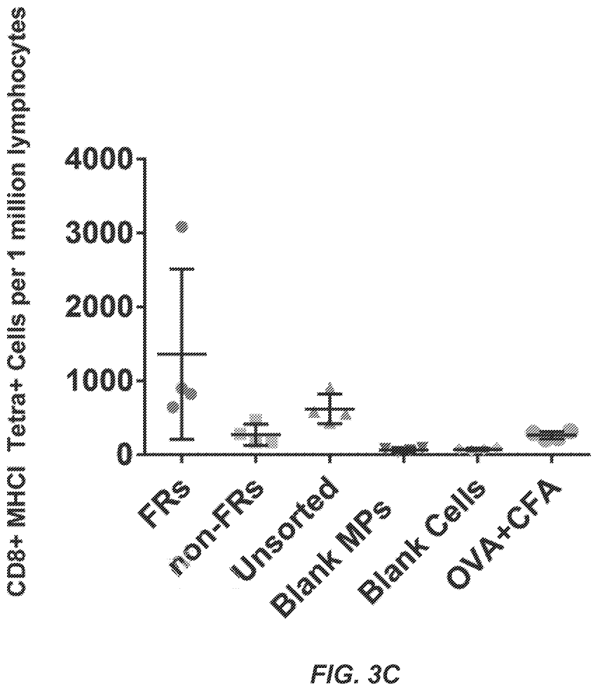

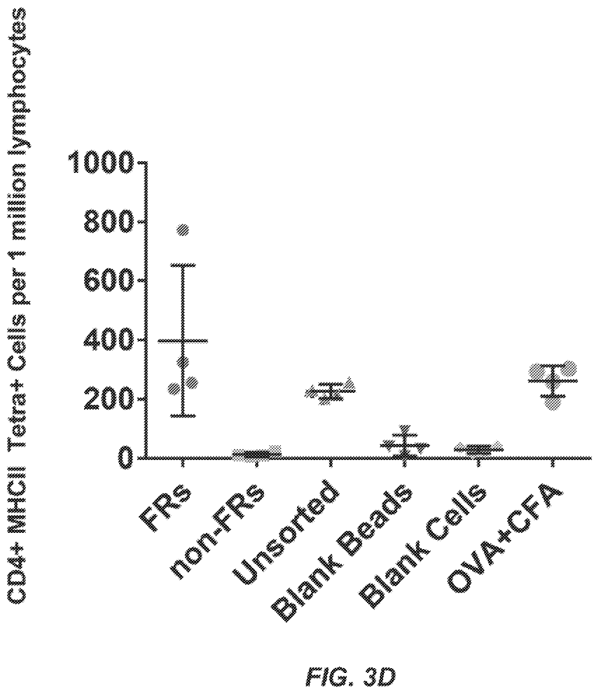

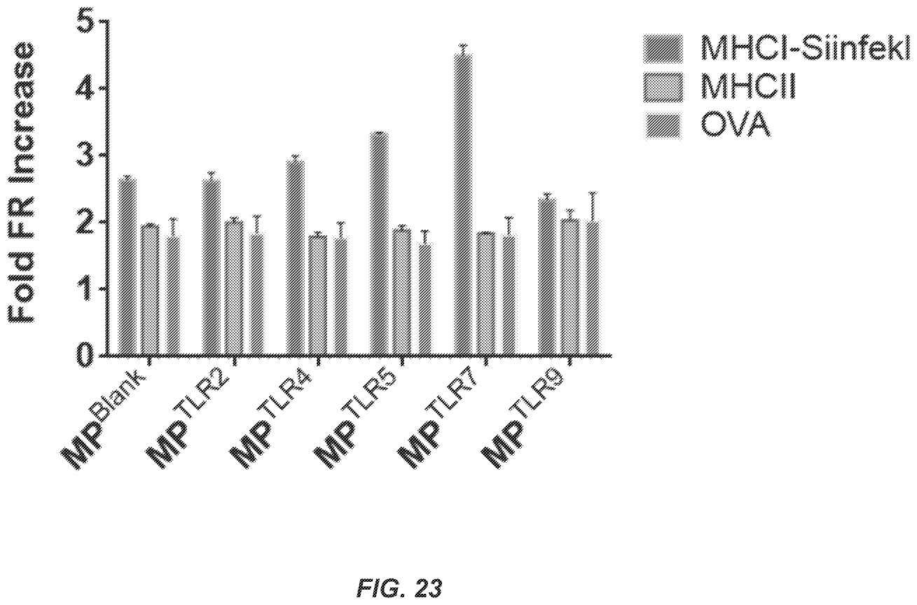

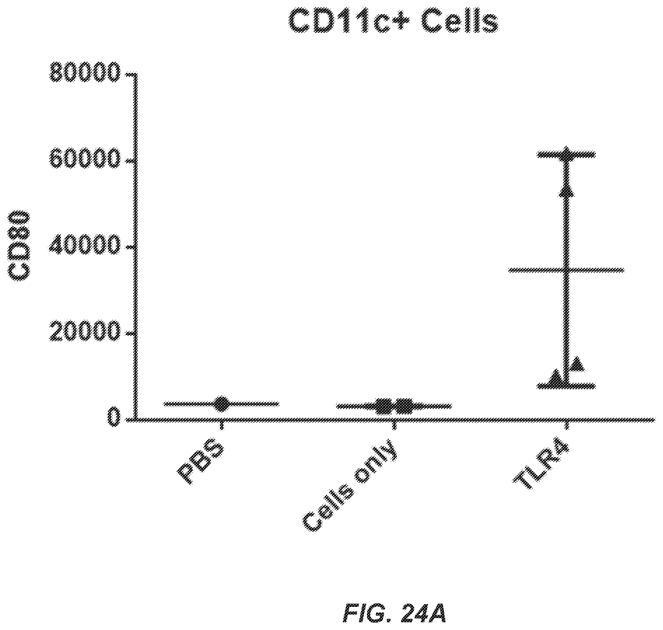

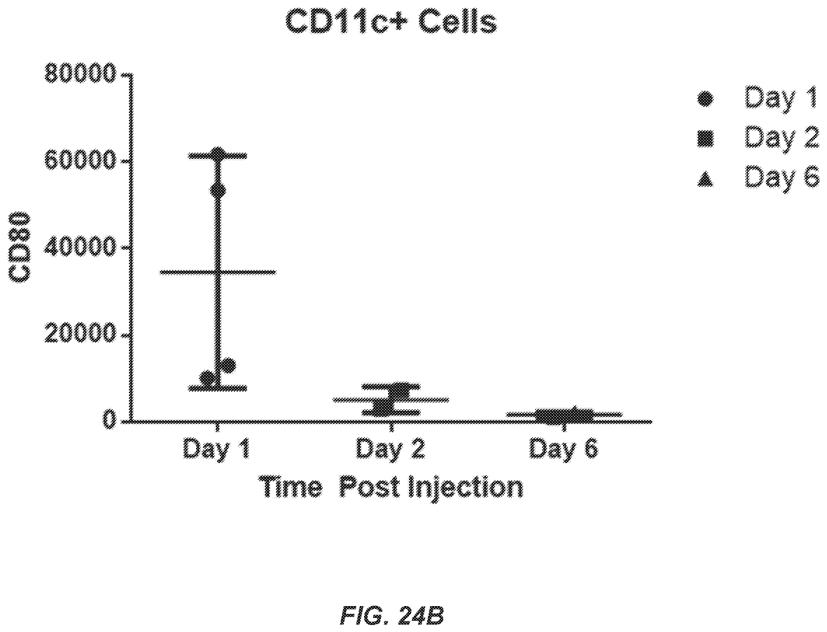

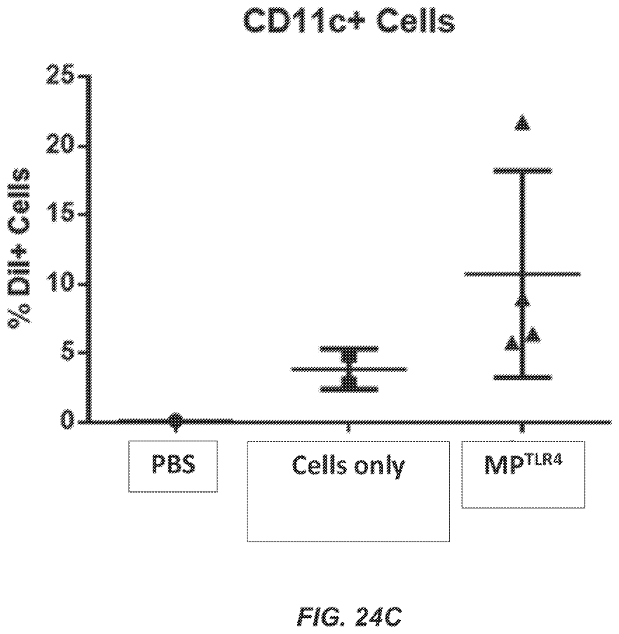

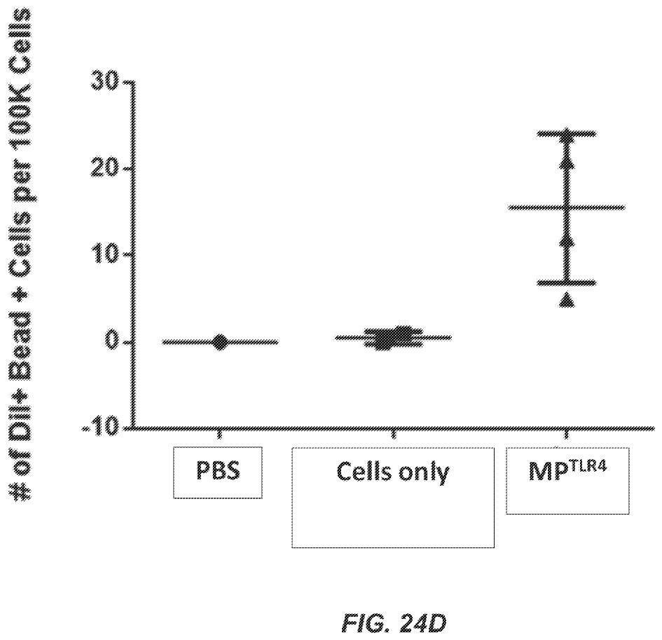

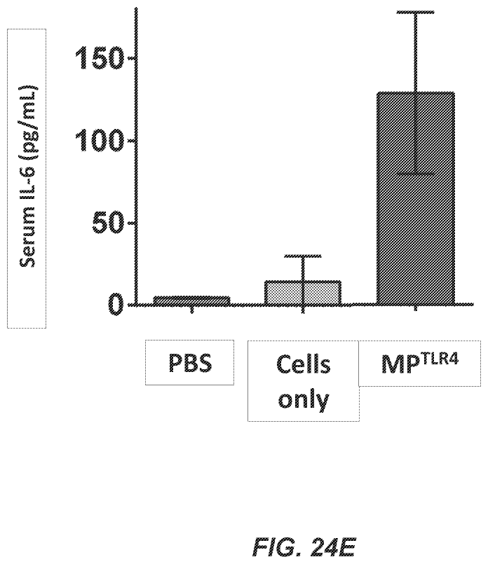

After determining how FRs coordinate innate immune activation and their kinetics, the inventors wanted to observe the impact of FRs on adaptive immune responses. To understand this further in vivo, the inventors performed an adoptive transfer of MP treated BMDCs with C57BL/6 mice in combination with the antigen ovalbumin (OVA). The inventors had observed in vitro that BMDC FRs have increased expression of MHCI and II and present more OVA, so the inventors hypothesized that they might also play a larger role in antigen presentation and formation of adaptive immune responses (FIG. 23) To make a preliminary assessment, the inventors compared if BMDC FRs could recapitulate part or all of the stimulation of an adaptive response of a larger collection of BMDCs in an adoptive transfer experiment. The inventors hypothesized that if the FRs were controlling a larger portion of the response, then removing the other cells would have a minimal impact on the final adaptive response. BMDCs were incubated with MP^{TLR4} on a 1:1 cell/MP basis for 30 minutes, washed and then concentrated at 30 million cells/mL (or 3 million in the case of FRs) with 10 μg/mL of OVA. To accomplish this goal, FR isolated and washed from a larger population of BMDCs were injected into both mouse footpads at 1 million cells per footpad for unsorted group or nFRs and at 100 k per footpad for FR groups (FIG. 3A). All experimental groups were treated with OVA except for PBS controls. The inventors previously determined that BMDCs injected into the footpad migrate to the popliteal lymph nodes and confirmed that these cells initiate immune responses (FIGS. 24A-24E). After 14 days, adoptively transferred mice were sacrificed and their sera was sampled for OVA specific IgGs. Popliteal lymph nodes were disaggregated and cells analyzed.

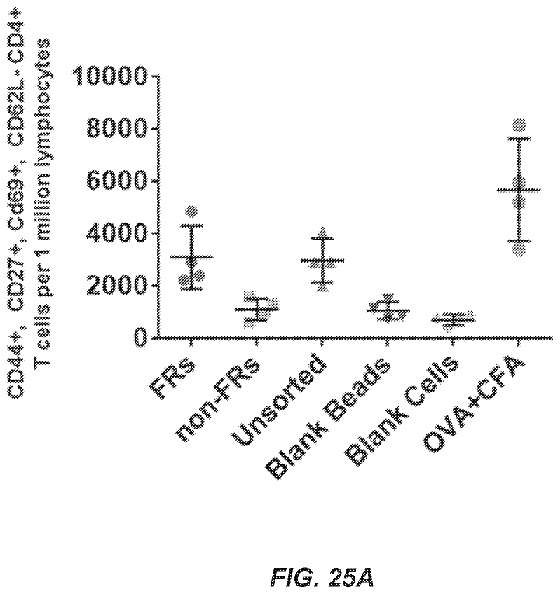

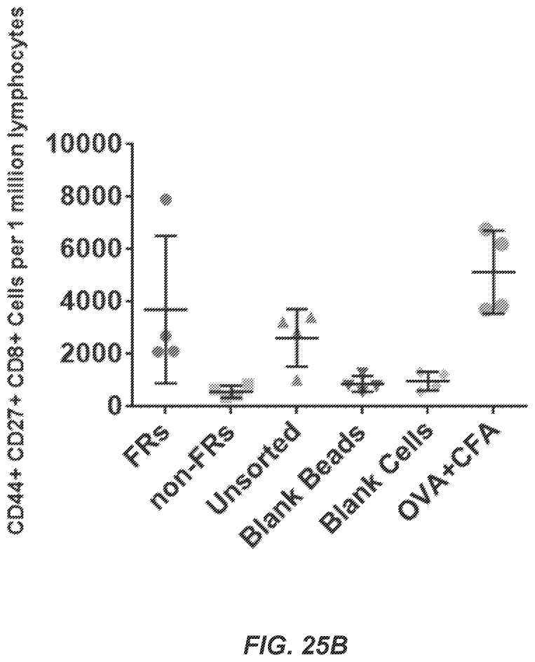

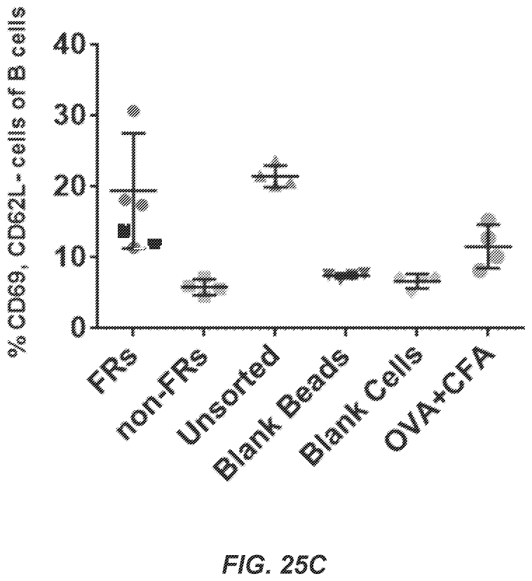

Mice injected with isolated FRs had a >10-time increase in anti-OVA IgG titers over mice injected with BMDCs not treated with MPs (Blank Cells) (FIG. 3B, p<0.05). This increase is particularly striking because FRs were injected with 10-fold fewer cells than the blank cell group and blank MP control group. Furthermore, when 1 million non-FRs were transferred in place of the 100 k FRs, very little OVA-specific IgG was measured. Adding to this, when the unsorted mixture of 1 million FR and nFR was transferred a response nearly equivalent to the FRs was measured, indicating that FRs are necessary to trigger adaptive immune responses in an adoptive transfer. To ensure that particulates did not influence antibody responses, identical controls were performed with MPs containing no TLR agonists prior to transfer resulting in no IgG production. Similar trends between treatment groups were also seen in the production of OVA specific CD4 T cells for both the major MHCI epitope (OVA 257-264) and the MHCII epitope (OVA 323-339) and for CD8 T cell responses (FIGS. 3C-3D, FIGS. 25A-25C).¹⁹ It is important to note that while FRs seem to be critical for BMDC mediated activation via adoptive transfer, the restriction of the APCs used to activate adaptive immunity may have reduced overall immune response. In contrast, a positive control using mice injected with OVA and Complete Freud's adjuvant (CFA) which stimulated only native APCs resulted in 10-fold higher IgG titers. From this data, the inventors concluded that in adoptive transfer experiments FR contributed an out-sized proportion of the response in antigen presentation and DC mediated induction of adaptive immunity given their very small population numbers pointing to the possibility of a large role for in vivo responses.

FRs have a Unique Temporal Transcription Profile

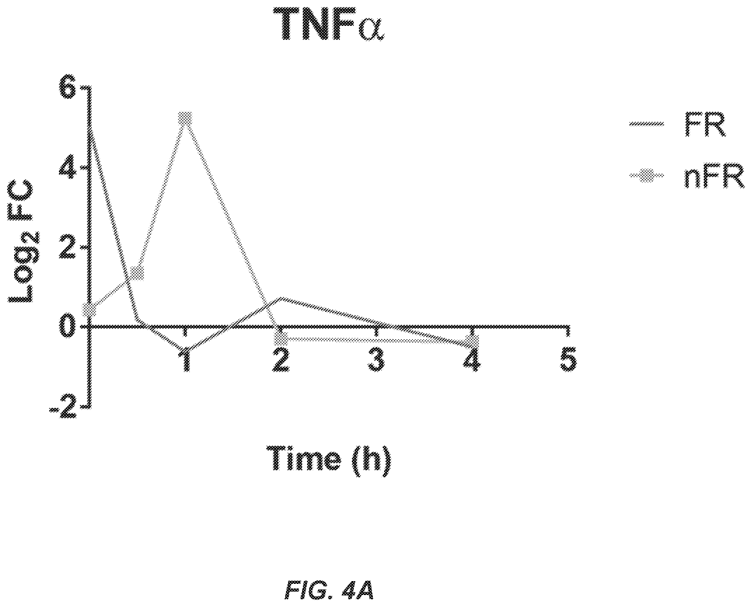

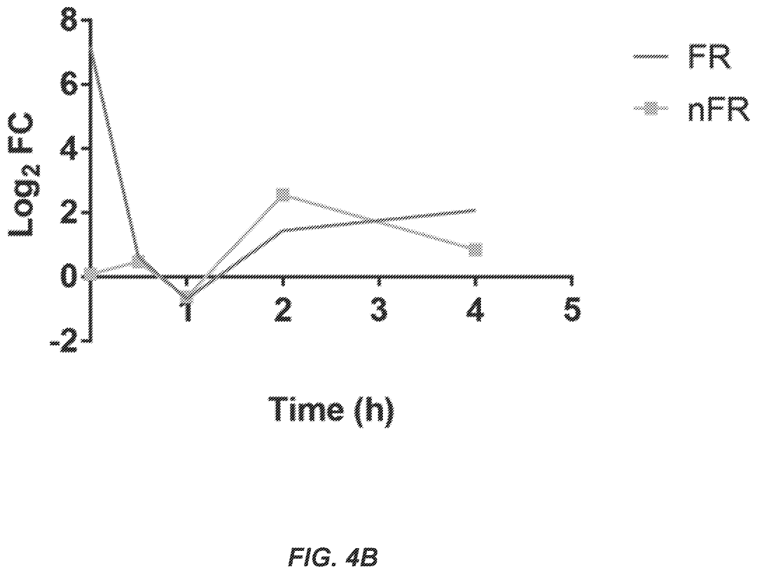

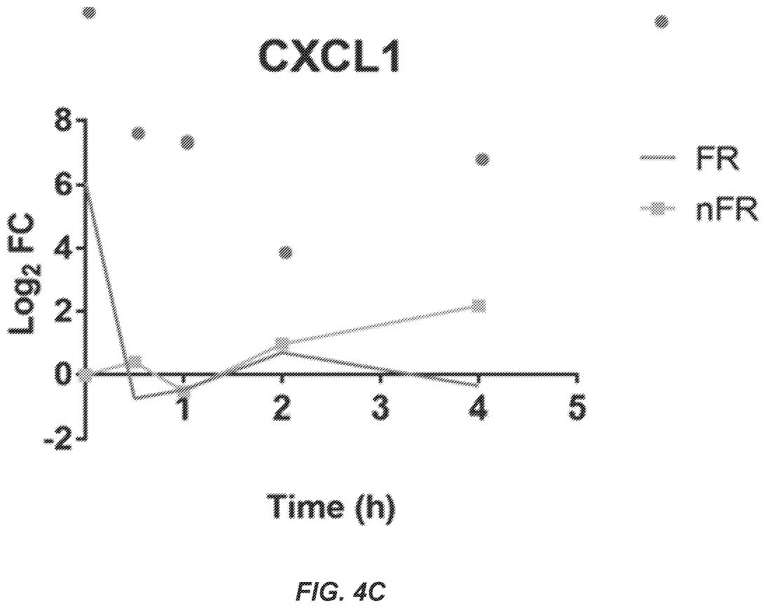

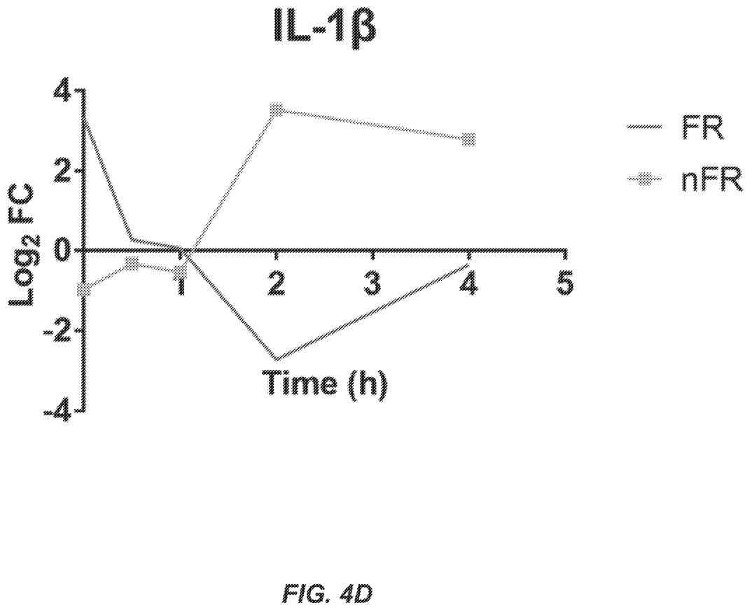

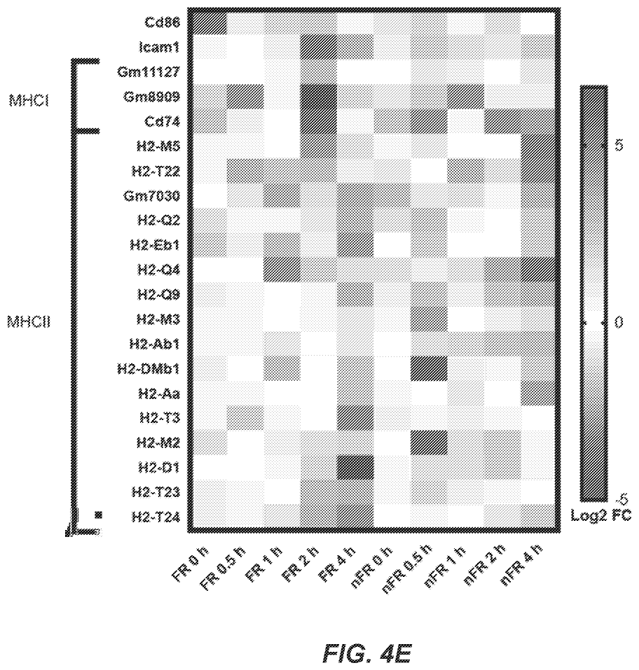

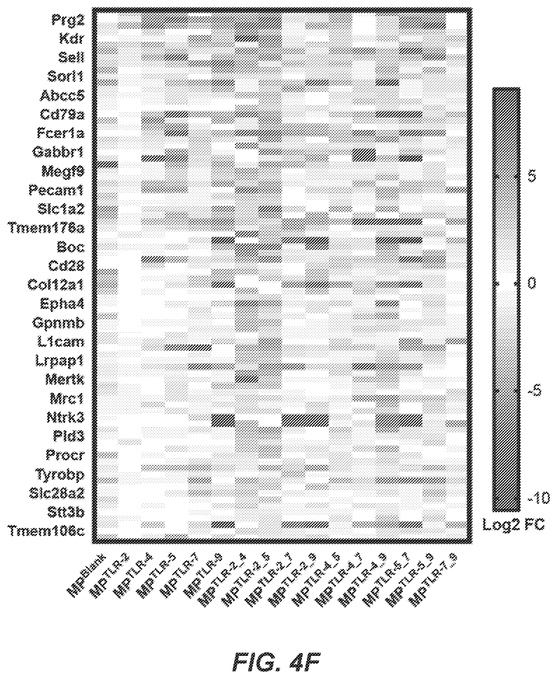

Given the importance of FRs for developing adaptive immune responses, the inventors sought to further characterize FRs activity via whole transcriptome sequencing. The inventors incubated BMDCs 1:1 with MP^{TLR4} for 15 minutes, washed, isolated FRs (the top 5% of MP signal) from the nFRs (bottom 90%) using FACS, and incubated for either 0 (immediately after sorting), 0.5, 1, 2 and 4 hrs and then isolated mRNA using a commercially available kit (Illumina) and sequenced the mRNA with the aid of the University of Chicago genomics core. The mRNA sequences were aligned to the mouse transcriptome and two-fold mRNA upregulation compared to an untreated BMDC control sample. The inventors noticed that at the 0 h timepoint, transcription levels for several inflammatory cytokines, such as TNFα, INFβ, CXCL1 and IL1β were highly upregulated in FRs but not upregulated in nFRs (FIGS. 4A-4D). In later timepoints, these cytokines mRNA levels decreased for FRs but increased for nFRs. For example, TNFα mRNA levels dropped to below baseline after 1 h for FRs but peaked for nFRs at 1 h (FIG. 4A). This further confirms the inventors' hypothesis that FRs have a burst release of inflammatory paracrine signaling cytokines immediately following TLR engagement, which simulates neighboring cells at later timepoints. It is surprising that this TLR-mediated immunity is then amplified so widely by the nFRs in such a short time. Also, of interest is that the timing of transcription response for the nFR more closely match the responses normally associated with a response to TLR stimulation for a population of BMDCs.²⁰

In addition to the cytokine expression, the inventors wanted to determine whether the primary function of the FRs immune response is limited to the quick cytokine burst, or if it extended to other aspects of a typical immune response, such as antigen presentation. A list of antigen presenting genes were acquired through the Mouse Genome Informatics database, and genes with 2-fold change in expression and pval <0.05 for at least one time point were analyzed. Overall, the inventors observed more upregulation for MHCI and MHCII genes in the FRs than in the nFRs (FIG. 4E). FRs upregulated antigen presenting genes at 2 and 4 hr while nFRs did not demonstrate upregulation. Likewise, the MHC co-stimulatory molecule CD86 is upregulated in 0 hr time point in FRs whereas no upregulation is seen in nFRs. Furthermore, as highlighted previously, the inventors saw a 2 fold increase in MHCI and MHCII protein expression in FRs 15 mins after sorting (FIG. 23). From this data, the inventors expect FRs to not only be responsible for bulk immune stimulation, but also to be important in the initial antigen presentation as well.

In order to find unique identifying proteins of the FR state, the inventors analyzed the transcriptional response for upregulated transcriptions of FR cells corresponding to potential surface proteins. One unique challenge for identifying surface protein upregulation in such a time-sensitive and dynamic cellular state as FRs is determining the appropriate timepoint for observing mRNA expression.²¹ In light of this, the inventors preliminarily incubated BMDCs with varying MPs for 16 hrs in Brefeldin A then sorted FR and nFRs and isolated mRNA. This timepoint was chosen for two reasons: (1) brefeldin A has been shown to cause mRNA accumulation over time and (2) while it is unlikely that mRNA for already expressed proteins would be upregulated at the time of sorting, the inventors hoped to observe upregulation on the next FR “cycle”.²² Additionally, the inventors did not want the unique identifying proteins to depend on the TLR agonist. Thus, the inventors incubated BMDCs with MP^{TLR-X}, where x is various single or dual TLR agonist(s) attached to the MP for 16 h and isolated the FRs and nFRs for sequencing via FACS (e.g., x=2 indicates a TLR2 agonist, x=2_7 indicates a TLR2/7 agonist). To find unique proteins, the inventors looked for the genes that were most consistently upregulated amongst these dosing conditions (2-fold increase in expression and pval <0.05) when comparing the transcriptional profiles of the FRs to the nFRs. The data showed upregulation of several surface proteins of interest and while further study would be necessary to identify an optimal timepoint, this provided a list of potential targets of over 25 potential proteins for FR identifying markers (FIG. 4F). These proteins are provided in Table 2.

TABLE 2
FR identifying markers
Protein Marker

Rcbtb2
Tyrobp (PRG2)
DAP12
CLC5A
TREM2
Cd300lb
F5
Ephx1
Entpd1
Hnmt
Lpar6
Serhl
Adgre5
Adcy7
Clec4a2
Gm8730
Npm1
Lyz1
Rpl5
Pck2
Gm13394
Msmo1
Fxyd2
Rps6
Rpl18a
Eef1b2
Qpct
Pcna
Cdk1
Cd300ld
Rnf13
Rpl12
Sgpp1
Rpl4
Slc30a5
Eif4b
Ank
Tmco1
Cd72
Rpl27a
Ogt
Kcnj2
Paics
Sesn1
Gm9625
Fcgr3
Slc50a1
Cd200r1
Txnip
Tmem273
Gm15772
Arhgap18
Rgs18
Prrc1
Stmn1

### FRs Express Unique and Identifiable Surface Proteins

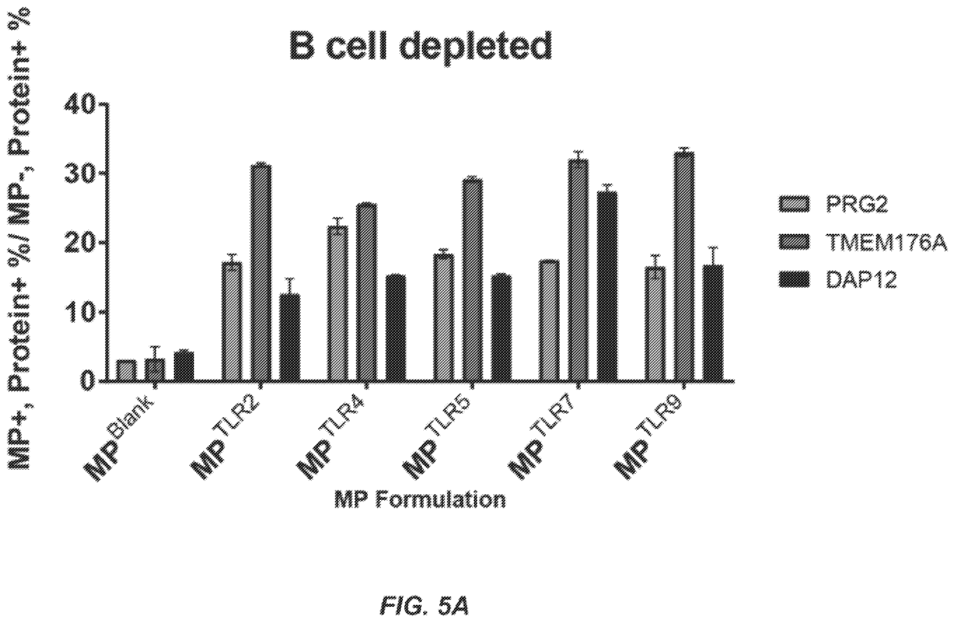

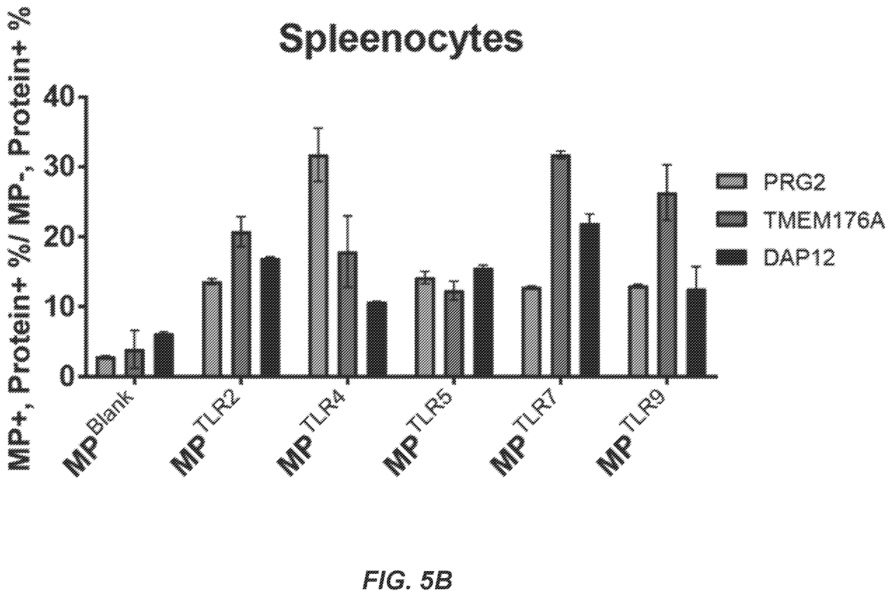

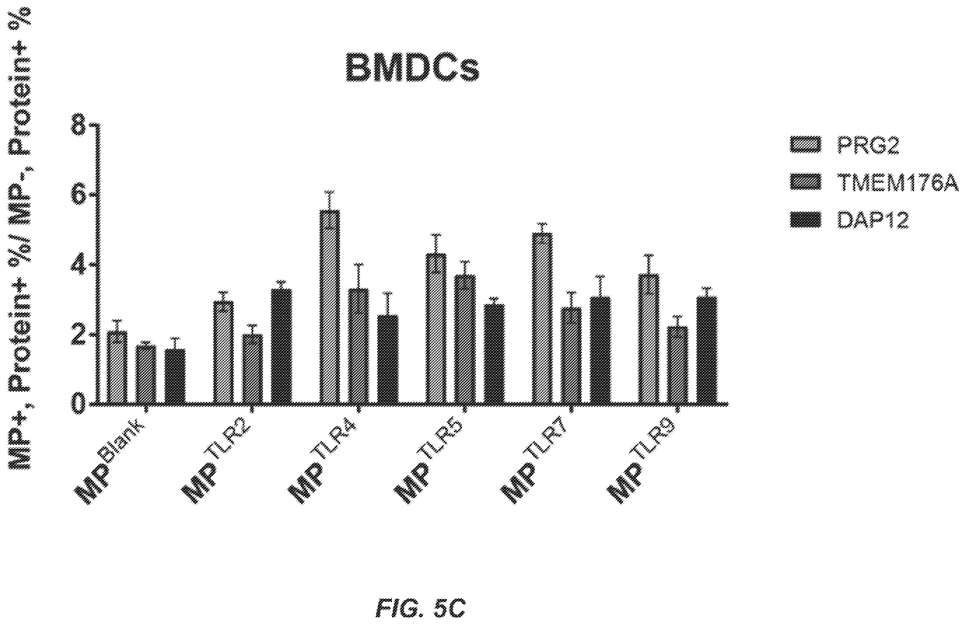

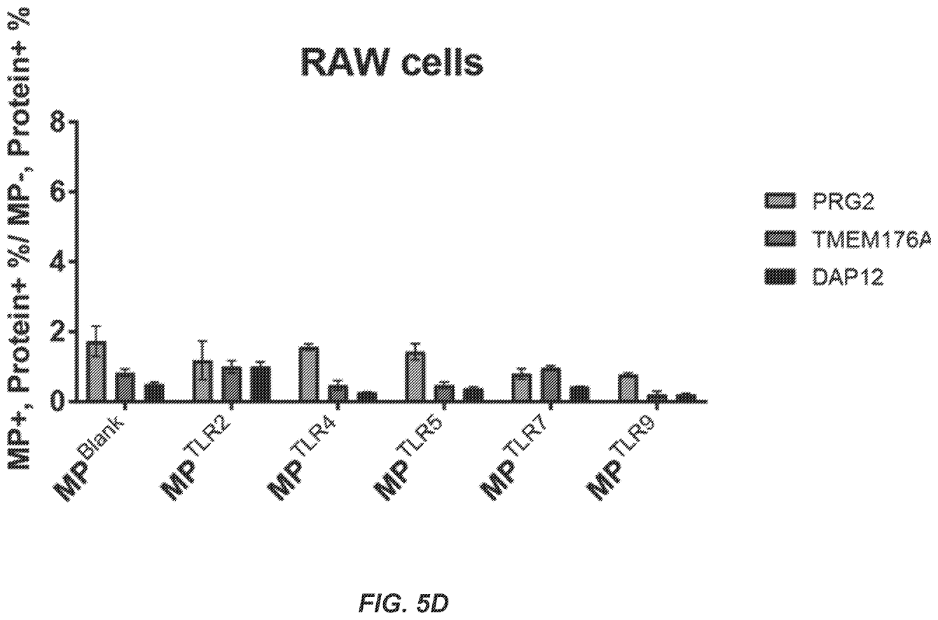

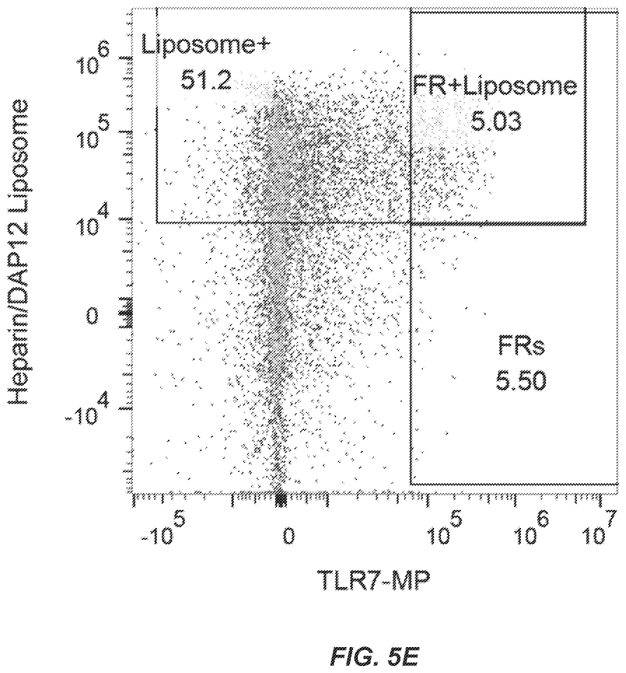

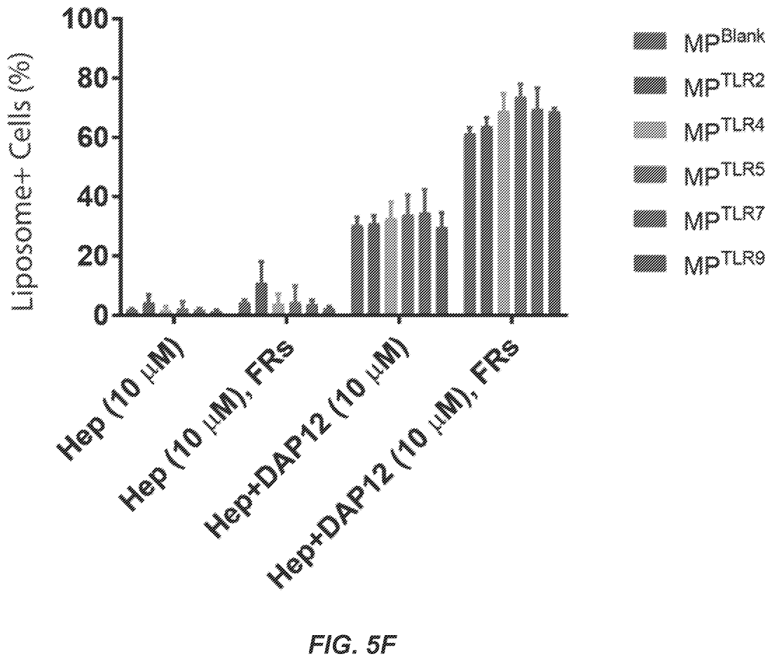

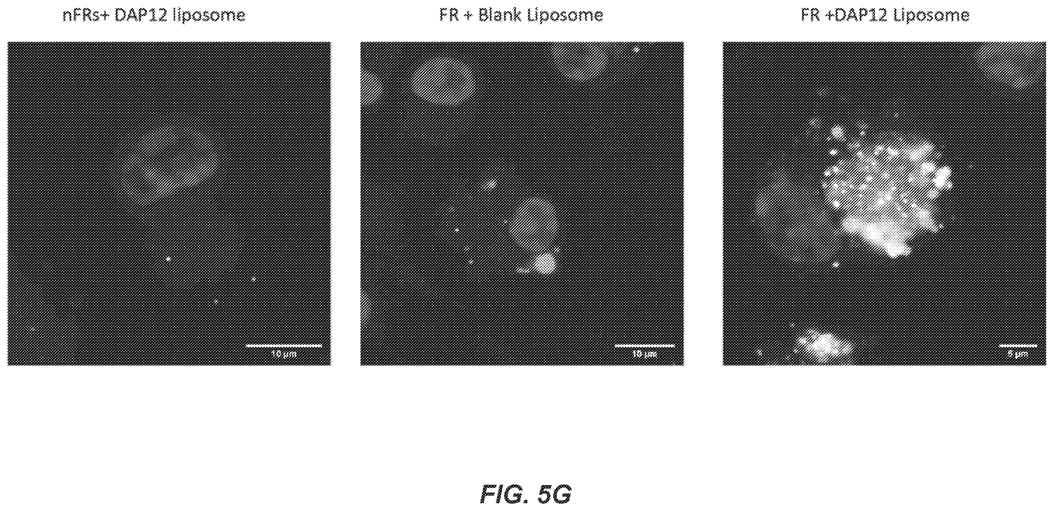

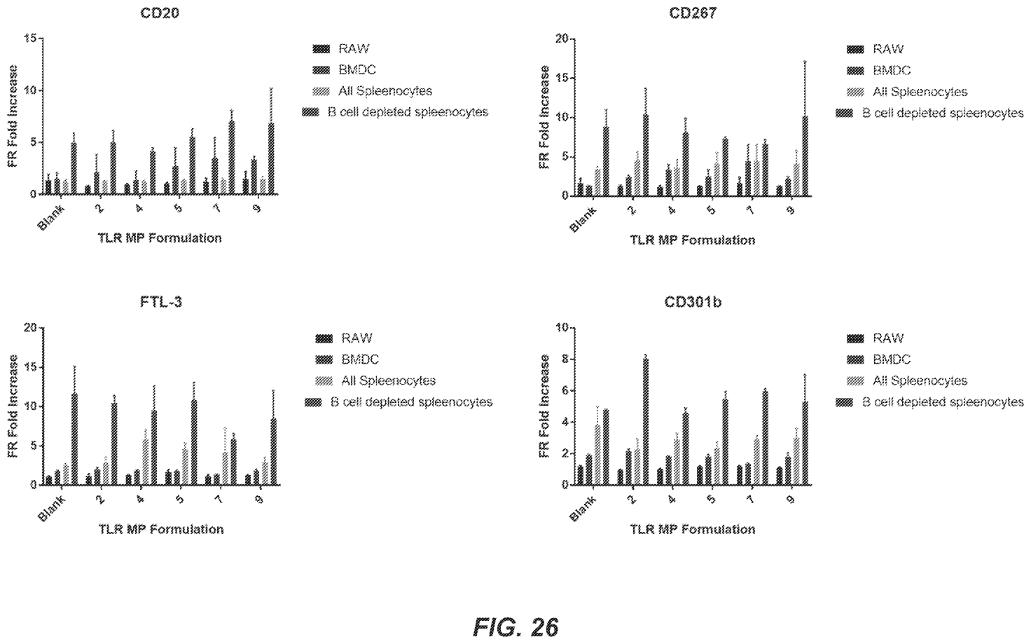

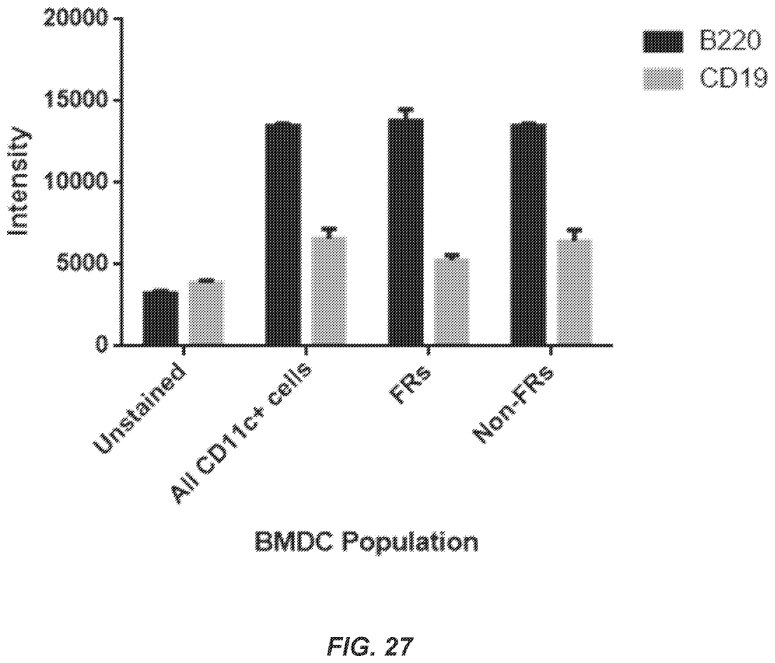

One initial limitation in the inventors' identification of FRs was the lack of identifying cell-surface markers—allowing them to be isolated from other DC cells. This problem is compounded by their transience and low-percentage within the cDC2 and DC population. To identify candidate protein for identifying FRs via a flow cytometry panel, the inventors selected proteins: (1) whose mRNA was upregulated in FRs compared to nFRs using all or most of the MP formulations, (2) were surface expressed and had commercially available high affinity antibodies, (3) had published low expression in other cells types and (4) were well characterized in the literature. The inventors tested potential candidates using flow cytometry, incubating with MPs for 15 mins to isolate FR using the inventors' established method then washing and staining for candidate markers to see test if the candidate markers identified the same cells. Using BMDCs and the inventors' mRNA analysis, the inventors narrowed the many candidates that were highly upregulated to just a few by applying a second set of conditions including eliminating non-surface markers or markers that are present in many types of immune cells such as CD20 or FTL-3 (FIG. 26). Given the presence of some mRNA corresponding to what are canonically considered B cell markers, the inventors also validated that FRs do not express actually common B cell markers such as CD19 and B220 (FIG. 21). From this selection, the inventors identified three markers of interest, DAP12, PRG2 and TMEM176A, that were upregulated 3-7 times in FRs vs nFRs in BMDCs (FIG. 5A).

Furthermore, they were even more highly upregulated in mouse spleenocyte CD11c+ cells and in B cell depleted spleenocyte CD11c+ cells (FIGS. 5B-5C). Importantly, there was not a high fold change in control samples using a homogeneous cell line (RAW), confirming that the observed increase of expression is not an artifact of the inventors' analysis but rather due to the heterogeneity of the cell population themselves (FIG. 5D).

Cells in the FR state were also more heterogeneous and dynamic that the inventors first anticipated. There appears to be increasing levels of upregulation of DAP12, PRG2 and/or TMEM176A depending on the TLR agonists conjugation to the MP. Unsurprisingly MP_{Blank} had the lowest fold increase, but there were very high increases for PRG2 with MP^{TLR4} and DAP12/TMEM176A with MP^{TLR}7. This result suggests there may be different subpopulations of the FR state which correlate with different TLRs or TLR agonists, although further research would be required to validate this hypothesis.

FRs can be Targeted by their Surface Proteins

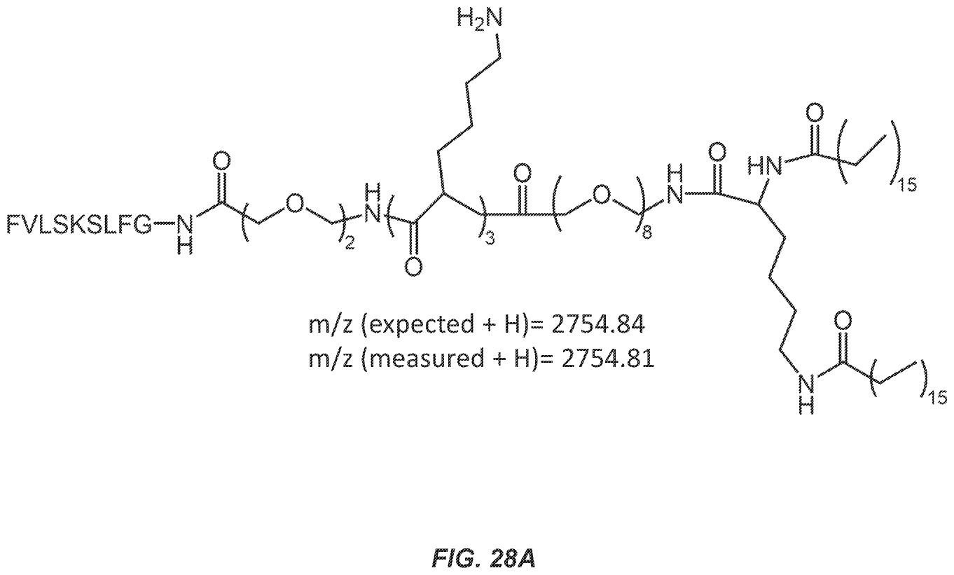

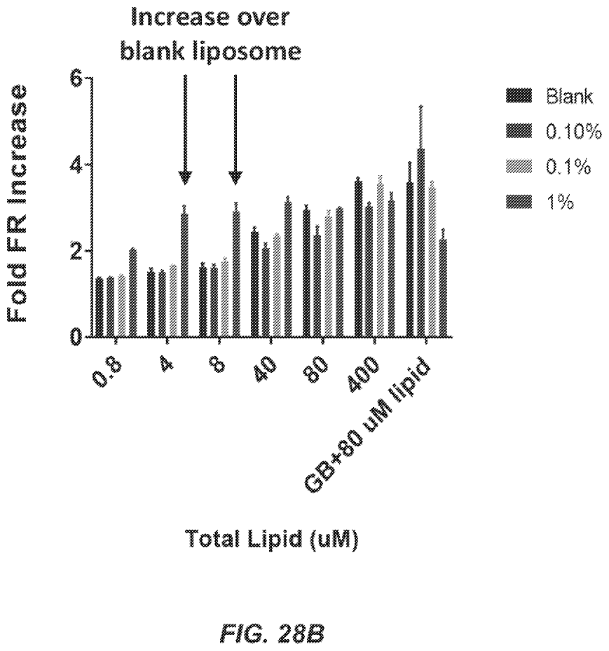

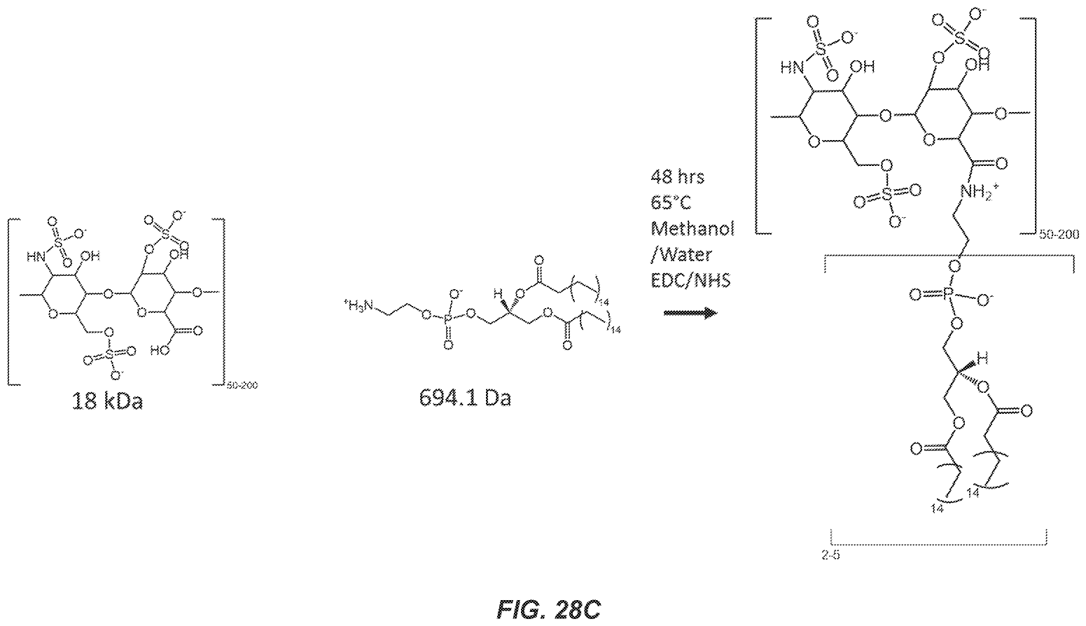

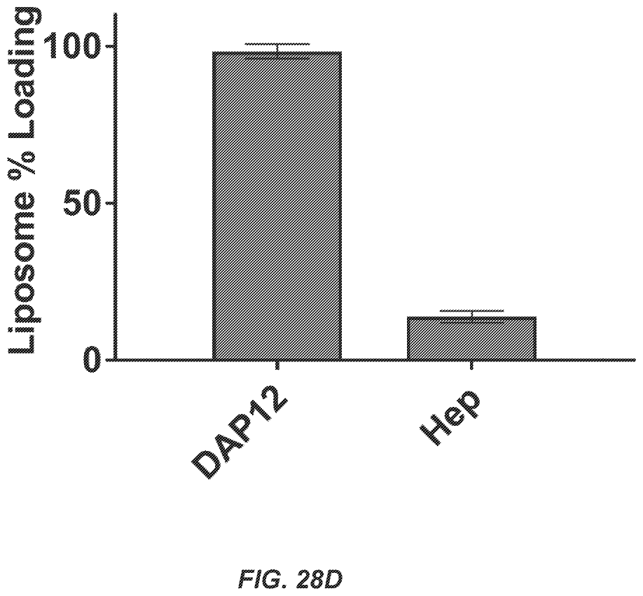

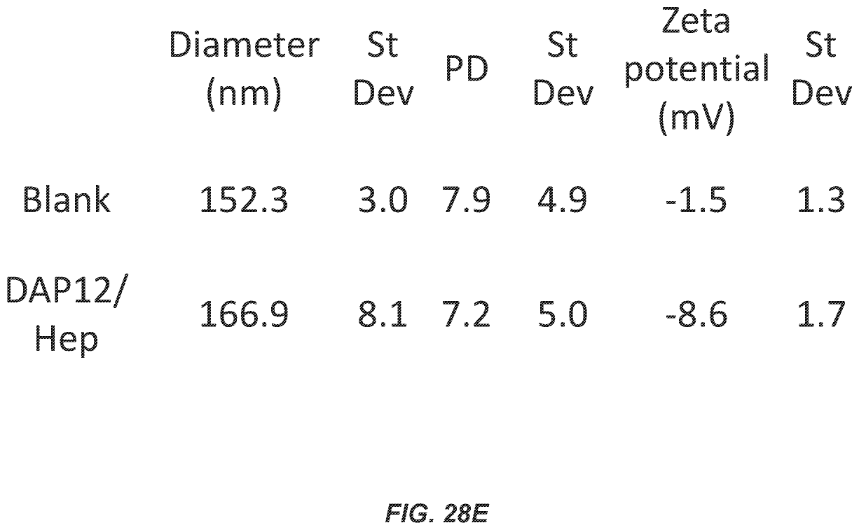

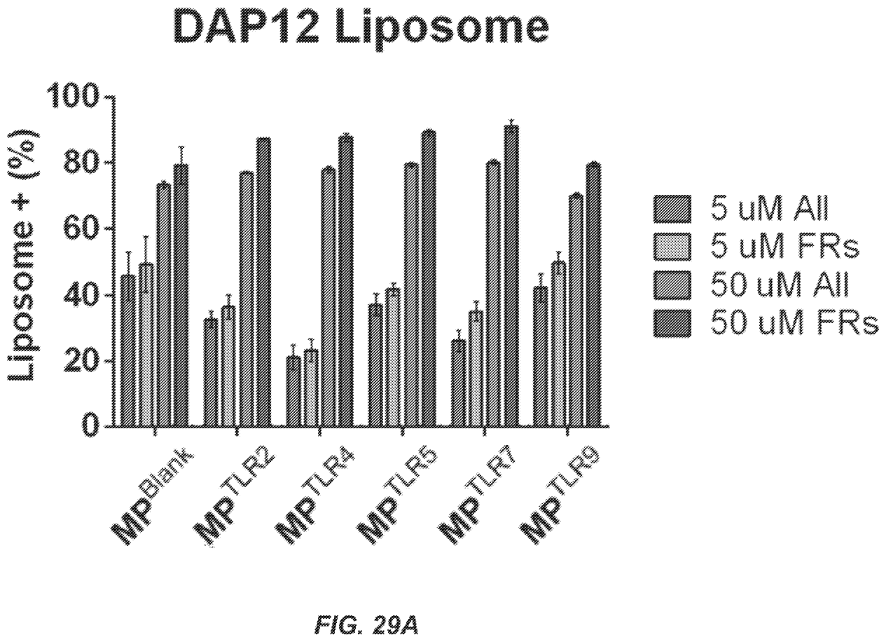

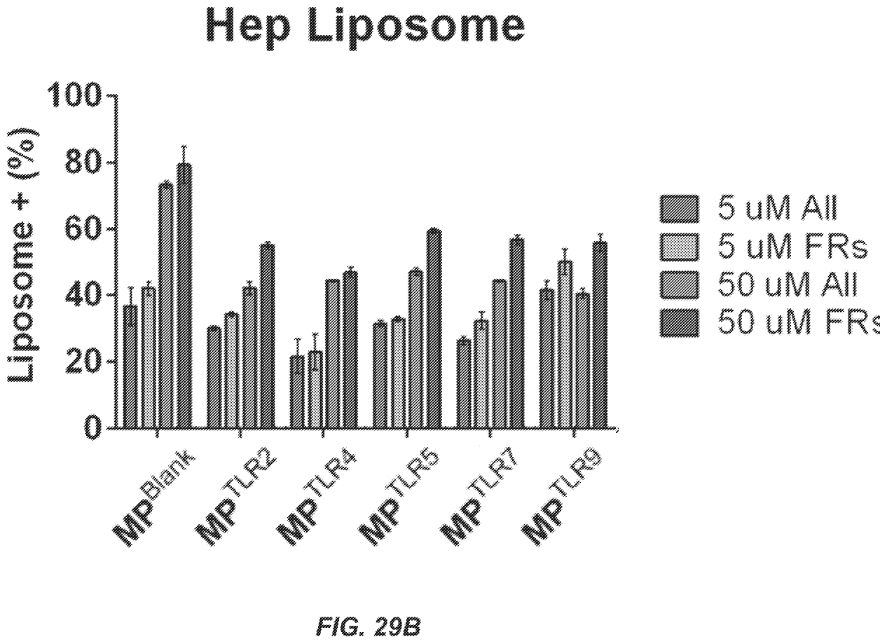

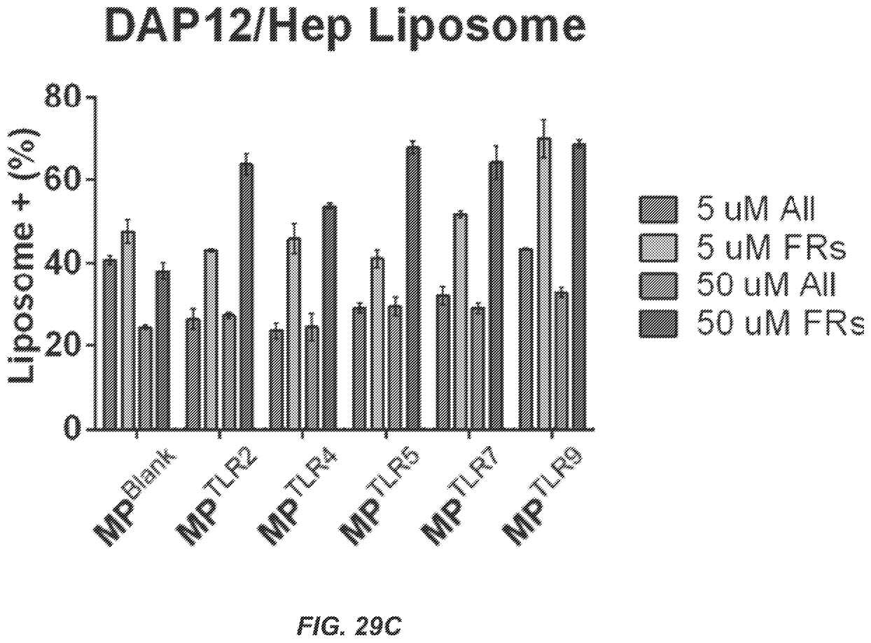

After confirming that the FRs both had unique uptake, markers, and contributed to a large degree of an adoptively transferred BMDC stimulation, the inventors sought to examine how much of an in vivo response was mediated by FR state. To accomplish this, the inventors would need a method to target and ablate the FR state during the short period when each cell was transitioning through it. To target the FRs, the inventors used a multivalent approach, combining ligands for several of the markers the inventors had identified through flow cytometry. To bind DAP12, the inventors searched the literature and found a selectively binding nonamer peptide, GFLSKSLVF. To bind PRG2, the inventors identified heparin which has moderate affinity.^{26,27} In order to test if these targeting elements would identify FRs, the inventors used a well characterized liposomal system to insert both a DAP12-binding peptide and Heparin conjugated lipid into 200 nm DSPC liposomes. The inventors synthesized and purified lipid conjugated versions of the DAP12 and heparin polymer (FIG. 28).^{28,29} The inventors then generated liposomes using the membrane extrusion technique (200 nm filters, see methods for additional details on liposome synthesis and composition). By inserting the targeting elements during formulation, the inventors generated several ratios and compositions of the two targeting elements. The inventors determined that the optimal ratio of targeting elements in the liposome was 10% heparin-lipid and 1% DAP12-peptide lipid (FIG. 28). The inventors then coincubated a fluorescently labeled version of the heparin/DAP12 liposome with BMDCs or spleenocytes for 15 mins, and washed to remove excess liposomes. After staining cells with potential FR-identify liposomes, the inventors incubated cells with TLR conjugated MPs for 30 minutes, washed and observed uptake via flow cytometry for MPs and liposomes. (FIGS. 5E-5F). Only when the liposomes contained both heparin and the DAP12 targeting peptide GFLSKSLVF (SEQ ID NO:1) did the inventors observe a significantly higher percentage of liposome positive cells for the FR population, indicating that targeting both receptors was necessary (FIG. 29). This was also observed via confocal microscopy, where BMDCs that had high MP signal also had high liposome signal only in the targeted formulations but not for BMDCs with no MP uptake or BMDCs incubated with blank (non-targeted) liposomes (FIG. 5G). This data indicates that FR cells can be reliably targeted with the heparin/DAP12 peptide liposome formulation and that the cells targeted with this formulation match the population the inventors previously identified via the inventors' microparticle experiments.

### In Vivo Validation of FR Ablation and Targeting

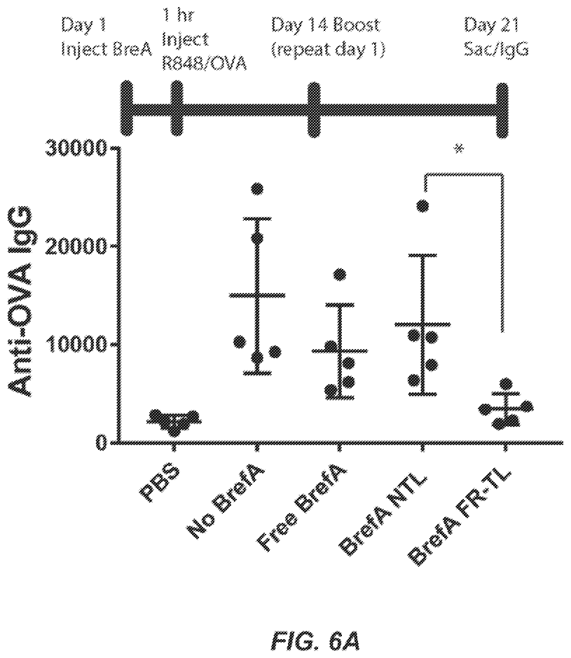

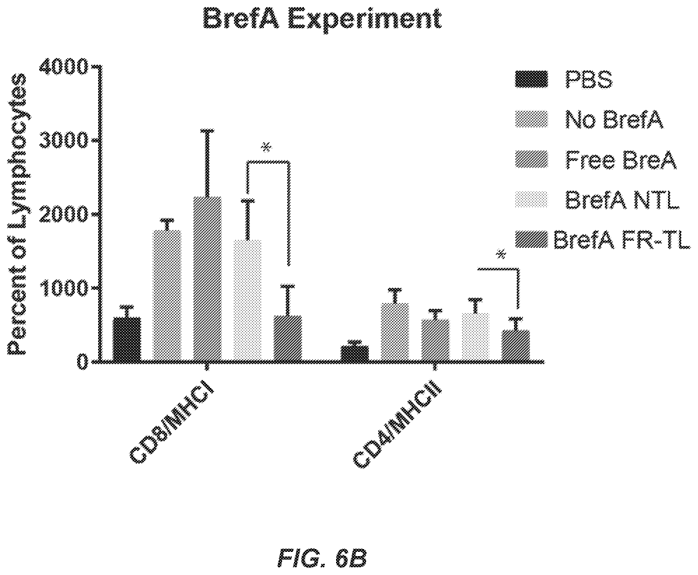

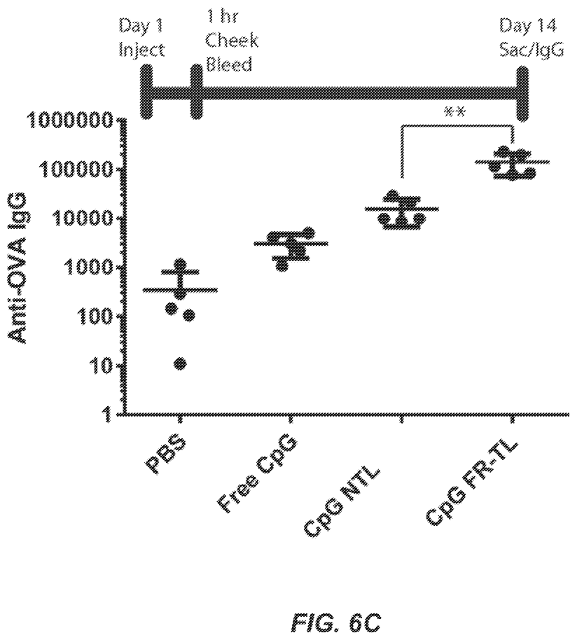

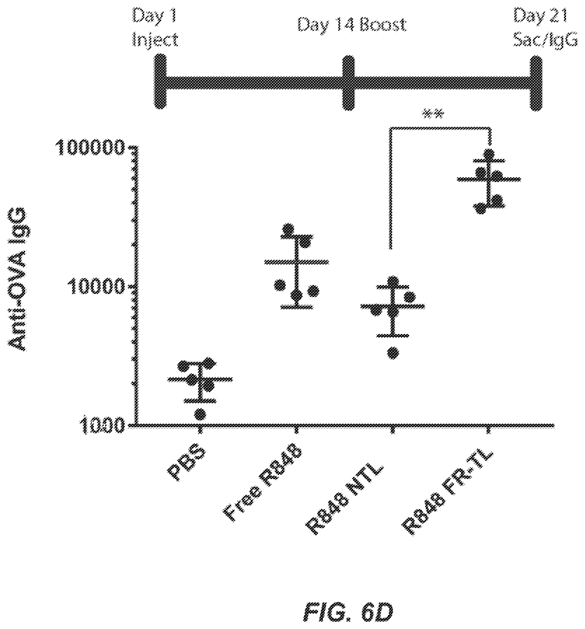

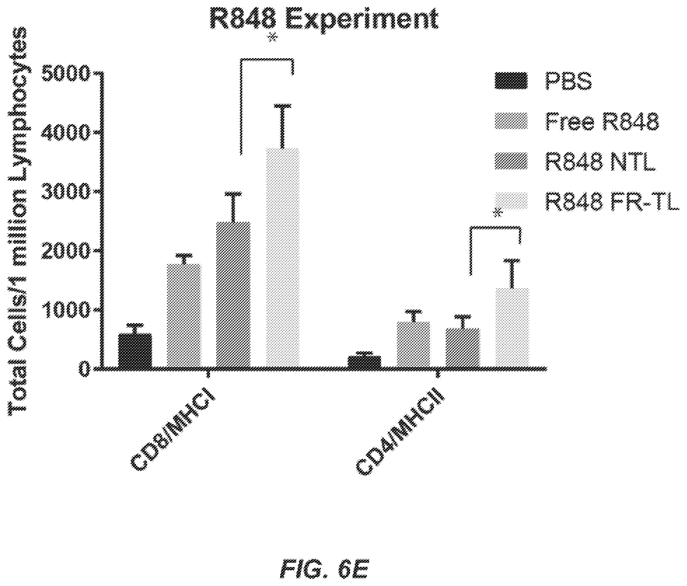

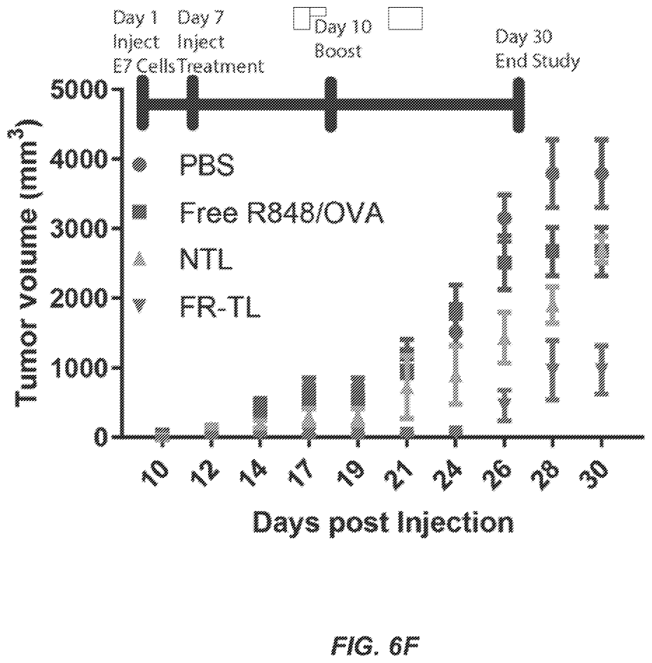

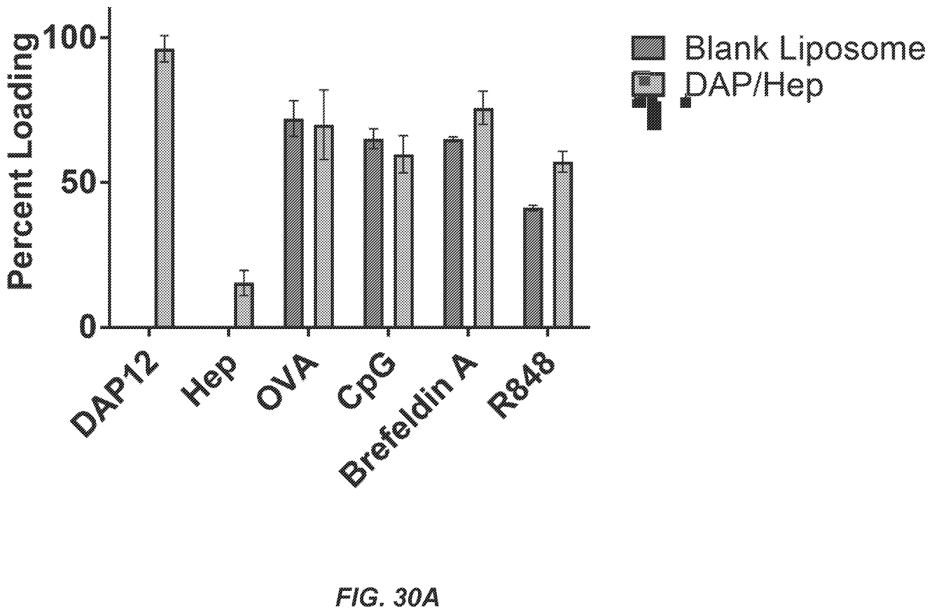

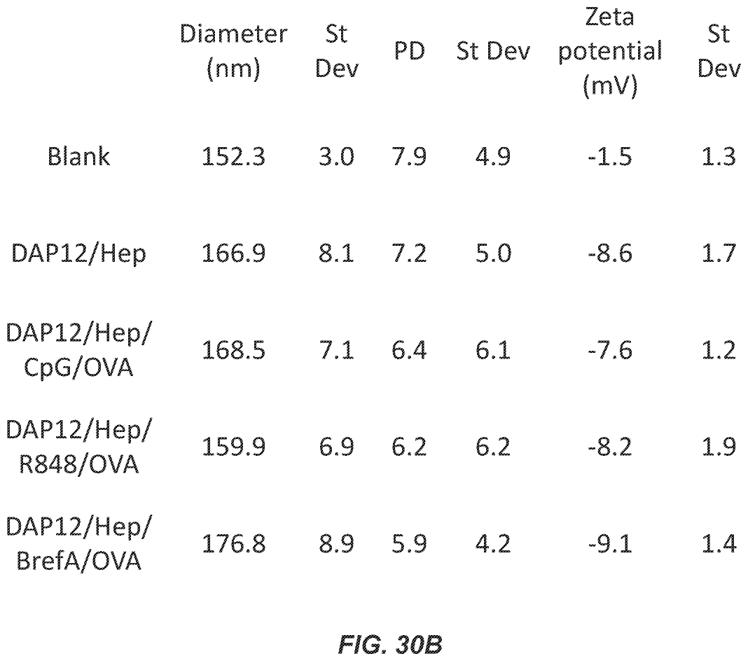

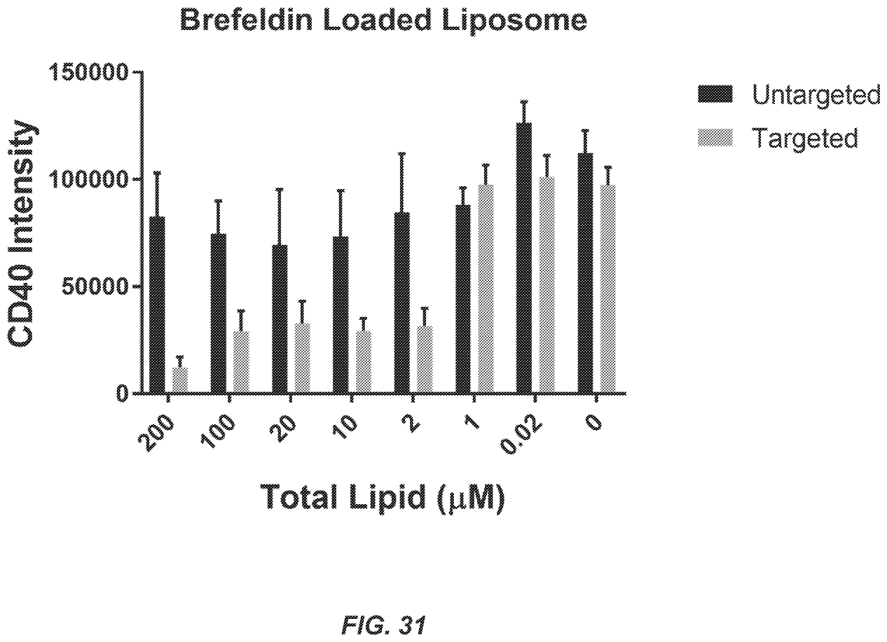

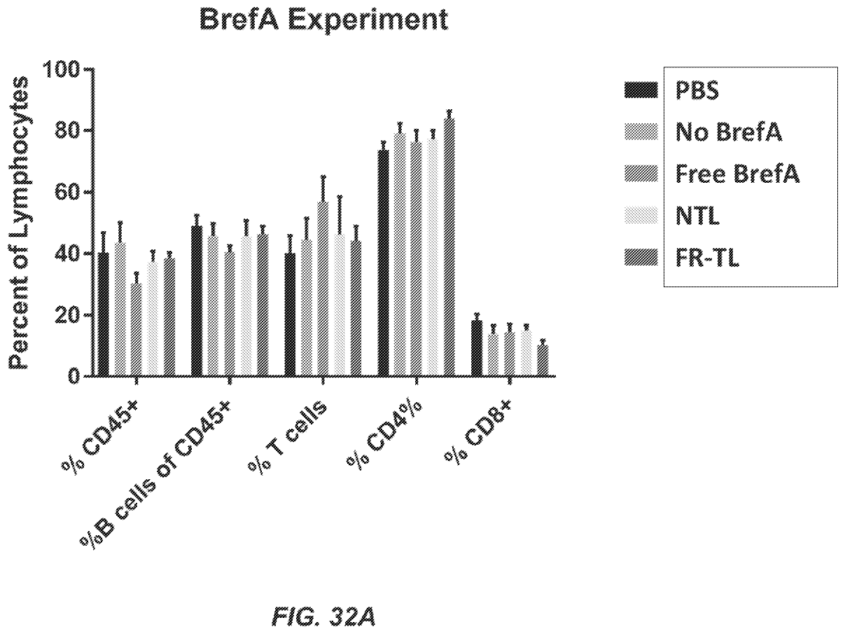

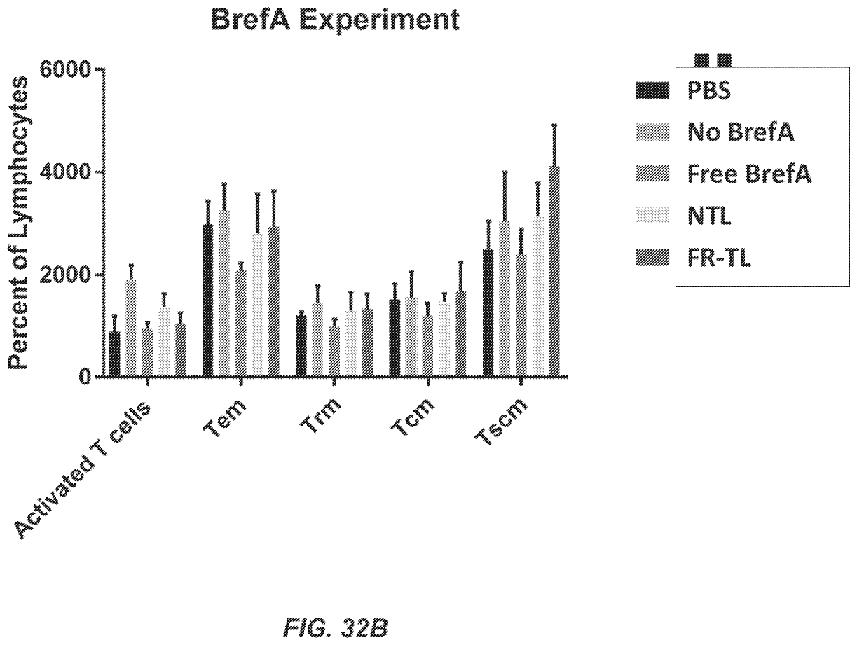

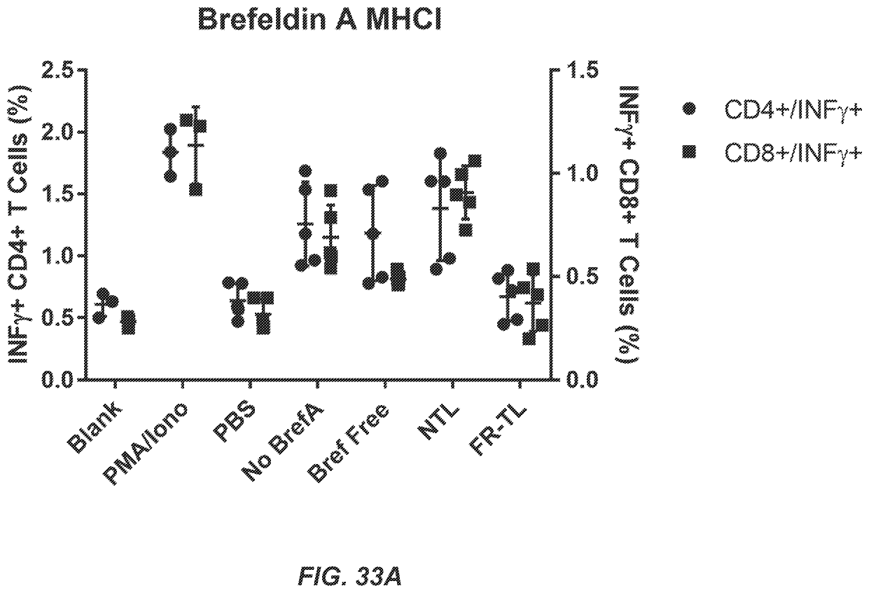

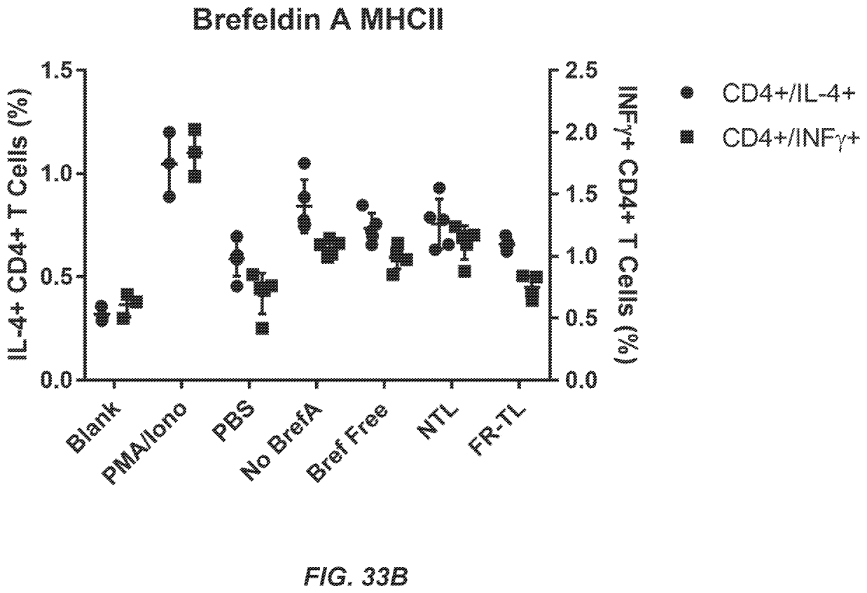

After demonstrating that FRs can be reliably targeted via DAP12/Hep loaded liposomes, the inventors sought to evaluate how this targeting effects adaptive immune responses. The inventors wanted to confirm that ablating the activity of the FR state would lower the adaptive immune response. However, as the FR state is transient and only exists, by the inventors' estimate, for 30 mins, this proved a difficult task. The inventors designed an experiment which would temporarily suppress the FR responses by delivery the protein trafficking inhibitor, BrefA (brefeldin A) the inventors had validated in vitro, with the liposomes—temporarily preventing FRs from signaling via paracrine signaling. The inventors loaded brefA at 1 mg/ml into 200 nm liposomes and observed an approximately 50% loading in both FR-targeted liposomes (FR-TLs) and non targeted liposome (NTL) and validated that these liposomes suppress BMDC activation in vitro (FIGS. 30 and 31). After validation, the inventors injected these brefA formulations into mice (100 μg per mouse, i.p.) and then 1 hr later mice were injected i.p with 10 μg of R848 and 100 μg of OVA (free formulation). Controls included liposomes without BrefA (No BrefA), an equivalent free quantity of BrefA (Free), and liposomes with BrefA, but no targeting agent (NTLs). On day 14, mice were boosted using identical conditions. Mice were then sacrificed on day 21 and their anti-OVA titers were analyzed. The inventors observed significant decrease in anti-OVA titers for the FR-TLs compared to NTL (FIG. 6A). Furthermore, the FR-TL mice also had decreases in their in CD8/MHCI+ and CD4/MHCII+ cells when compared to untargeted lipo (FIG. 6B). However, there was no significant change in CD45+ cell populations or any other lymphocyte cell population (FIG. 32). There was a significant decrease in INFγ+CD4 and CD8 T cells in the ICS analysis in response to the MHCI epitope but not to the MHCII epitope (FIG. 33). This data indicated that by inhibiting the FR population in vivo, the adaptive immune responses can be selectively diminished.

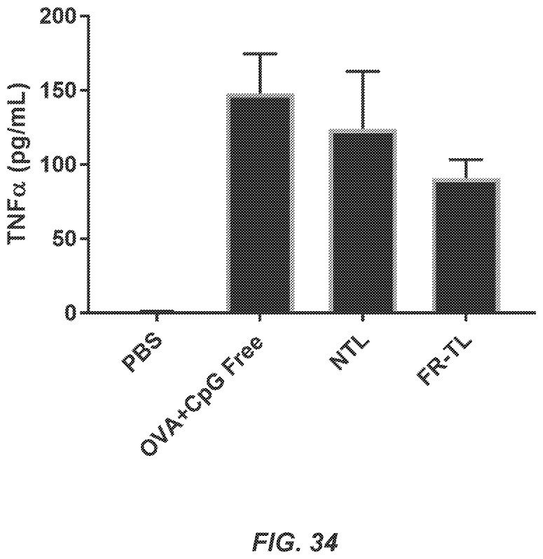

After demonstrating that FRs played a critical role in vivo, the inventors were intrigued and wondered if the FR-targeted liposomes could be used to enhance conventional responses to adjuvants. The inventors decided to observe the effect of FR targeting on the model antigen ovalbumin (OVA) in three separate experiments. First, the inventors loaded OVA (1 mg/mL) and CpG (a TLR 9 agonist, 200 μg/mL) into FR-TL or NTL (FIGS. 33A-33B).³⁰ The inventors injected these liposomes or an equivalent amount of free CpG/OVA i.p. into C57BL/6 mice (100 μg OVA, 10 μg CpG per mouse). On day 14, there was a nearly 10-fold increase in anti-OVA IgG titers CpG FR-TLs) compared to NTL (FIG. 6C). Interestingly, when the mice serum cytokine levels were measured at 1 hr post injection, the inventors did not observe an increase in TNFα levels, indicating that the FR targeting did not increase the systemic cytokine release (FIG. 34).

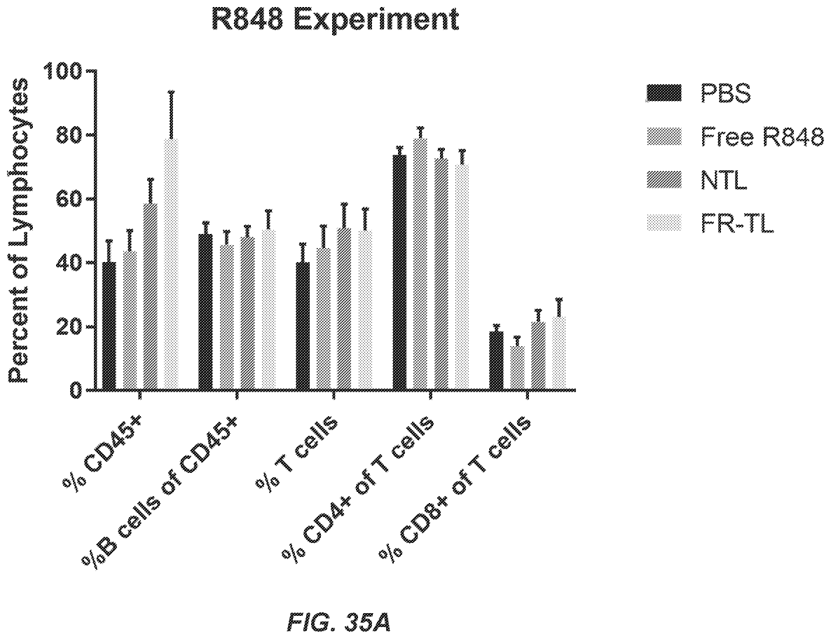

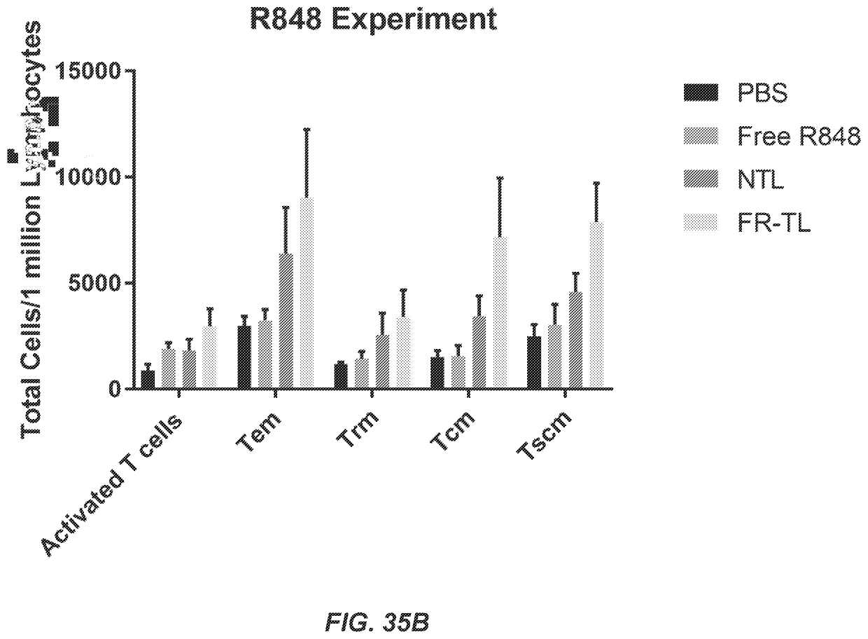

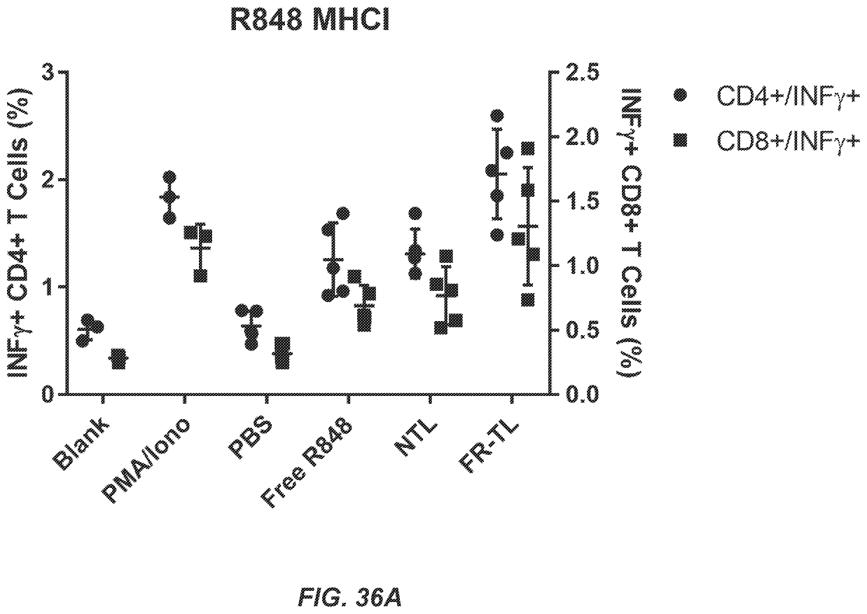

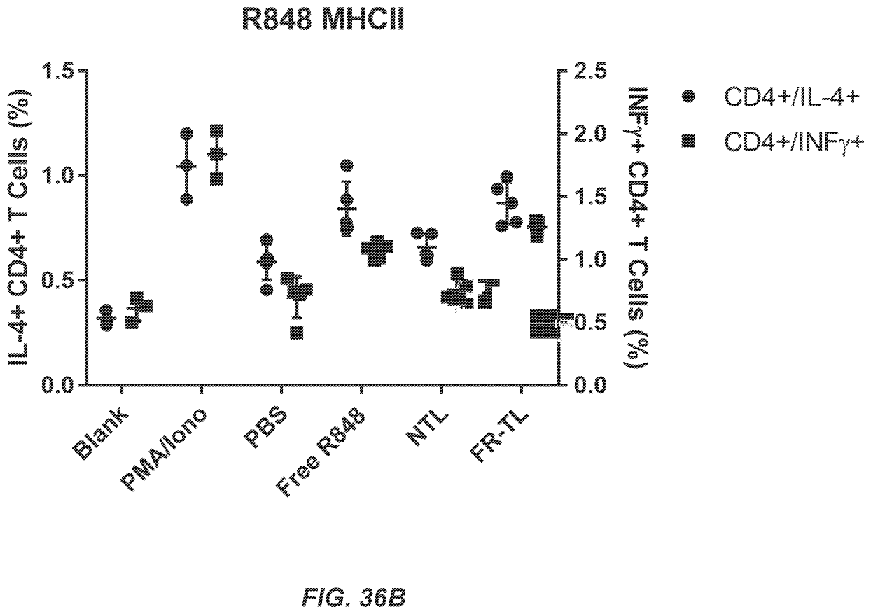

As the FR state had been responsive to multiple TLRs previously, the inventors explored if the FR-targeted liposomes changed the TLR agonist from CpG to R848, a TLR 7 agonist (FIGS. 28A-28E).³¹ C57BL/6 mice were injected with R848/OVA formulations on day 0 and 14 and then sacrificed on day 21. The data shows that FR targeting significantly improves anti-OVA IgG levels on day 21 even when changing the TLR agonist and the experimental design (FIG. 6D). Furthermore, popliteal and ingunial lymph nodes were removed from both sides of the mouse on day 21, homogenized, stained for various immune cell markers and tetramers to the major MHCI and MHCII epitopes and analyzed via spectral flow cytometry. As shown in FIG. 6E, targeting FRs significantly increases the number of both MHCI tetramer positive CD8+ T cells and MHCII tetramer positive CD4+ T cells. This increase seemed to be a result of an overall increase in CD45+ cells into the lymph nodes and not a disproportionate increase in T cells (FIG. 35A). Furthermore, there was an increase in various T cell subtypes in the FR-targeted group, indicating that FR targeting increases overall cell proliferation instead of a particular cell subtype (FIG. 35B). The spleenocytes from these mice were also analyzed via intracellular staining (ICS) to observe the responses of T cells ex vivo when stimulated by exogenous OVA peptides. There were significant increases in INFγ expressing CD4 and CD8 T cells in responses to the major MHCI OVA peptide and an increase in both INFγ and IL-4 expressing CD4 cell in responses to the major MHCII OVA peptide in the FR targeted group when compared to untargeted liposomes, indicating a broad increase in CD4 and CD8 as well as Th1 and Th2 responses, although the increase in Th1 responses to the MHCI epitope was more dramatic (FIGS. 36A-36B).

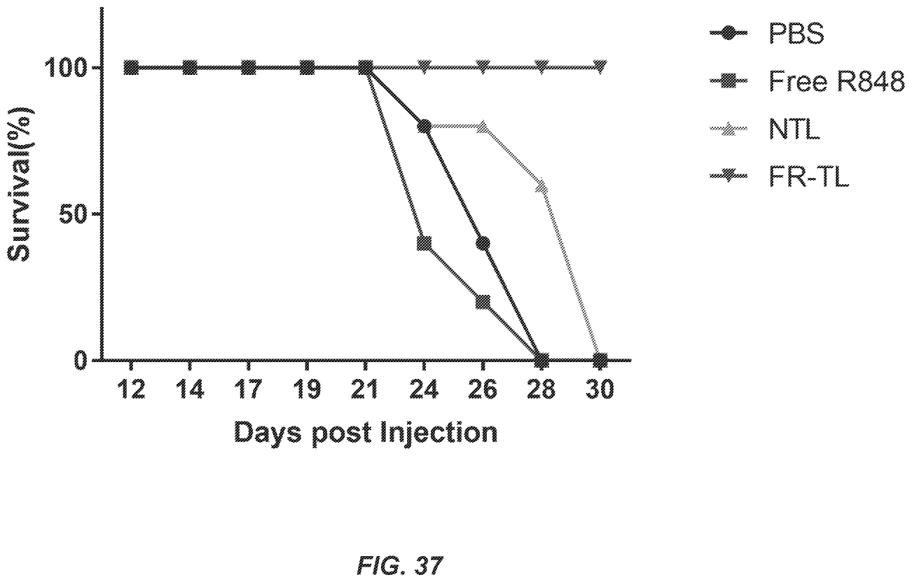

Finally, as the inventors saw increases in many parts of the immune repsonse, the inventors wanted to determine if the antigen-specific responses elicited by FR-targeting could be translated into therapeutic models. To test this, the inventors used a OVA expressing tumor model (EGF7.OVA) to show that FR targeting alters immune responses in a disease model.³² The inventors hypothesized that due to the increase in MHCI CD8 T cell responses from FR targeting, a stronger anti-tumor response would be observed. The inventors injected C57BL/6 mice (n=5) with 2×10⁵ E7-OVA cells on day 1 followed by injections of an R848/OVA vaccine formulations or a PBS blank on day 7 and day 10. Tumor volume was measured over a 30 day period during which the mice sacrificed when tumor reached >20 mm in any direction in accordance with the inventors' protocol. There was a significant reduction in tumor volume for both liposomal formulation when compared to PBS controls beginning at day 14, but there was a further decrease in tumor volume for the targeted formulation compared to the non-targeted which continued for the remainder of the study (FIG. 6F, p<0.05). For example, on day 24, the FR-TL group had an average tumor volume of 70±37 mm³, while all other groups had an average tumor volume >800 mm³. On day 30, all mice except for the FR-TL group were sacrificed due to tumor size (FIG. 37). Taken together, these experiments indicated that FR targeting improved overall immune responses in vivo by increasing CD8 T cell and IgG responses.

### Materials and Methods

### Materials

All chemicals were purchased from Sigma unless noted. Lipopolysaccaride from E. coli (LPS), Flagellin from Bacillus subtilis (FLA), Lipoteichoic acid from S. aureus (LTA), R848, CpG 1826, Ovalbumin (OVA) were purchased from Invivogen. The TLR7 agonist 2BXY was synthesized in house according to published protocols.¹ Thiol functionalized CpG 1826 (GpG-SH) was purchased as a custom order from Integrated DNA technologies. BCA assay kit was purchased from Thermo. Cytoperm/CytoFix Kit plus GolgiPlug was purchased from BD Biosciences. All antibodies were purchased from BD Biosciences. All peptide synthesis reagents were purchased from EMD Millipore. Quanti Blue assay solution was purchased from Invivogen. Anti-OVA IgG ELISA kit was purchased from Alpha Diagnostic International.

### Methods

Silica-silane coated Polystyrene Microparticle (MP) synthesis: PS MPs were synthesized and coated with a silica coating using a procedure from Moser et al.² Briefly, uniform, spherical, 2 μm diameter polystyrene microparticles were synthesized via controlled styrene polymerization. 2 g of polyvinylpyrrolidone, MW 40,000 and styrene (20 g), washed with NaOH and dried with MgSO4, was dissolved in EtOH (250 mL) and purged with nitrogen. AIBN (0.2 g) was added, the mixture stirred at 70° C. and 200 rpm for 24 hrs. Mixture was purified by centrifugation (5000 RPM for 5 minutes, followed by washing 3× in 30 mL of EtOH to remove residual monomer, initiator, and stabilizer). The surfaces of MPs were modified with reactive thiol groups via Pickering emulsion reaction. A mixture of Cyclohexane (45 mL), n-hexanol (10.8 mL), endotoxin free water (2 mL) and Triton X-114 (10.8 mL) were placed in a round-bottom flask and sonicated for 20 min. Particles (0.2 g) were added and the suspension was sonicated for 40 min. TEOS (400 μL) was added dropwise followed by 14 M aqueous ammonia (1.2 mL). The resulting solution was stirred for 30 min RT. Subsequently, 3-Mercaptosilane (200 μL) was added dropwise and stirred for 6 h. TEOS-mercaptosilane copolymer coated particles were then pelleted at 3400 rpm for 30 min and washed 3× with EtOH. Particles were dried at 70° C. and stored at 4° C.

TLR agonist Surface Functionalization: Thiol bearing MPs were functionalized with MPLA and Pam2 using thiol-maleimide chemistry. First MPs (5 mg) were swelled in ACN (500 uL) for 30 mins under sonication, then 1 mg of FITC in 500 uL of ACN was added for a final concentration of 1 mg/mL for 30 mins. MPs were then centrifuged for 1 min (5000 rcf), supernatant removed and washed 3× with PBS. For MPs conjugated with agonists to TLR2, 5, 7 and 9: FITC labeled MPs were dissolved in 500 uL of PBS, then 5 mg of Bismaleimide-PEG3 was added and allowed to sonicate for 30 minutes, then washed 3× in PBS. During this LTA, FLA, 2BXY or CpG-SH (0.1 mg/mL) was incubated for 5 mins with Trauts reagent (1 mg/mL) in 500 uL of PBS. After washing, maleimide bearing MPs were incubated with thiol functionalized with either Trauts+LTA, FLA, 2BXY or CpG-SH (500 uL) and sonicated for 30 mins, then washed 10 times, 3× with PBS, 4× with PBS with 0.1 tween 20, and 3× with PBS. For TLR9 MPs, 1 mg of LPS was dissolved in 400 uL of DMF and 10 ug of p maleimidophenyl isocyanate added and allowed to stir overnight under argon. Then PBS (400 uL) was added followed by 2,2′-(ethylenedioxy)diethanethiol (4 mg) and allowed to stir for 12 hrs. 100 uL of this mixture was added to the MP with 400 uL of PBS for 30 mins and similarly washed. After conjugation, all MPs were diluted in PBS+0.05% wt tween20 at 20, 50 and 100× and then number of MPs in 10 uL counted using flow cytometry to determine a concentration. MP solutions were diluted to a concentration of 1 million MPs per uL of PBS and stored at 4° C. until use.

Particle Concentration Determination via Flow Cytometry—The concentration of particle in a given volume was determined using flow cytometry (NovoCyte flow cytometer, ACEA Biosciences, Inc). After the final wash, MPs were rehydrated in 500 μL of PBS, then diluted down in PBS +0.01% wt tween 20 by 50, 100 and 200 fold. 10 μL of these dilutions were analyzed via flow cytometry in triplicate, gating on FITC signal to remove noise. The MP concentration in the stock was then calculated using linear regression from the three dilutions. MP stocks were then diluted down with PBS to a final concentration of 1 million MPs/μL.

Synthesis and Purification of DAP12 targeting peptide lipid The peptide-lipid for liposome encapsulation was adapted from a paper by Stefanick et al.³ Synthesis was performed using a Liberty Blue™ automated peptide synthesizer using the schematic in Figure S-17. Rink amide resin (100-200 mesh, 0.55 mmole/g, 0.05 mg) was weighed out into a solid-phase peptide synthesizer reaction vessel. The peptide (FVLSKSLFG) was constructed with standard Fmoc protected peptides (0.2M in DMF) from the C terminus to the N terminus. Then a Fmoc-EG2-COOH was conjugated followed by three lysines, EG8 linker, a Fmoc-Lys(Fmoc)-OH and palmitic acid. Deprotection was performed using 20% piperidine in DMF. Coupling was performed after activation with diisopropylcarbodiimide (DIC) (0.5M in DMF) in the presence of Ethyl cyanohydroxyiminoacetate (oxyma) (1M in DMF). All other couplings were done at 90° C. for 5 min. All reactions and subsequent washes were performed in DMF. After the synthesis was completed, the resin was transferred into a Bio-Rad Poly-Prep chromatography column.

Global deprotection was achieved by agitating the resin in trifluoroacetic acid (TFA)/3,6-dioxa-1,8-octanedithiol (DODT)/triisopropylsilane (TIPS)/H2O (8.5:0.5:0.5:0.5) for 2 h. The peptide was precipitated by adding the cleavage cocktail filtrate to 30 mL diethyl ether in a 50 mL centrifuge tube pre-cooled to −78° C. The precipitate was collected by centrifuge (4000 XG for 5 min). The precipitate was dissolved in 50% CH₃CN in 0.1% TFA) and filtered through a 0.45 μm syringe filter. Purification was performed using reversed-phase HPLC C8 column (gradient elution with 30-90% CH₃CN/0.1% TFA over 20 min). Pure fractions were pooled together and the peptide was recovered through lyophilization and redissolved in MeOH. LC-MS (m/z) 2754.84 [M+H]+

Synthesis and Purification of Heparin Sulfate Lipid This procedure was adapted from a paper by Kim et al.⁴ 300 mg Heparin Sulfate (18 kDa) was dissolved in water (10 mL) and mixed with DPPE (60 mg) dissolved in IPA (10 mL). 138 mg EDC and 69 mg of NHS were added. The mixture was stirred constantly at 65° C. for 20 hrs. After reaction, the mixture was rotoevaporated to remove excess IPA, lyophilized, redissolved in 5 mL of water, filtered and dialyzed against water in a 3500 Da cutoff dialysis cassette for 48 hrs. Then the purified Heparin-lipid was analyzed via LC-MS, confirming between 2-5 conjugation of DPPE per heparin chain, lyophilized again and redissolved at 10 mg/mL in MeOH.

Liposome Synthesis Liposomes were synthesized via membrane extrusion method using a setup from Avanti polar lipids and 200 nm extrusion filters. DSPC, PEG2000 PE, Cholesterol, and/or Did, Heparin-lipid, DAP12 peptide lipid and any TLR agonists/Brefeldin A were combined in 1 mL methanol added, dried via lyophilization, and rehydrated in PBS to make a 10 mM total lipid, 200 uL solutions. OVA was added during rehydration. Solutions were gently rotated at 67° C. and then passed through a 70° C. 200 nm filter 5 times. The liposome solution was dialyzed against PBS with a 3500 Da filter for 24 hrs.

LC-MS Analysis of Liposome Loading Liposomes loaded with DiD, DAP12 peptide, Heparin, brefeldin A, CpG, R848, OVA or any combinations of these were tested for loading via LC-MS analysis. Stock solutions of liposomes were diluted down to 1 mM total lipid and then 5 uL of this solution was injected on a C8 analytical HPLC column with a gradient of 10-90% ACN in 20 mins and the effluent observed by an Agilent 6135BAR LCMS XT mass spectrometer and a diode array detector at 220 nm. Signal from various compounds in liposome formulations were compared to standard curves at 220 nm.

Dynamic Light Scattering Analysis of Liposomes Hydrodynamic diameter of liposomes was confirmed using a Wyatt Mobiu(DLS/ELS at 100 uM total liposome concentration.

SEM of Functionalized Particles: Scanning electron microscopy (SEM) and energy-dispersive spectroscopy (EDS) of the particles was performed using an FEI Quanta 3D FEG dual beam (SEM/FIB) equipped with Inca EDS (Oxford Instruments). High-resolution images were taken with an FEI Magellan 400 XHR SEM particle samples were dried under vacuum for 24 h, mounted on carbon tape, and sputter coated (South Bay Technologies) with approximately 2-4 nm of Au/Pd 60:40 or Ir.

Cell Culture RAW 264.7 Cells were cultured at 1 million cells per mL in DMEM+Penicillin/Streptomycin+10% HIFBS and split every 2-3 days.

BMDC Cell Culture BMDCs were cultured according to a previously published protocol. Cells were used between 5-7 days after isolation.⁵

Human moDC Culture—Frozen PBMC (BuyPBMCs.com) were purchased commercially and the vial of 10 million cells were warmed at 37° C. Then, PBMCs from the cryo-vial are transferred into the new tube contains warmed media (RPMI1640). Then centrifuge the cells at 400×G for 10 minutes. The supernatant was discarded, resuspended and centrifuged. CD14+CD16− monocytes were isolated by using the positive selection kit from StemCell Technologies (EasySep™ Human Monocyte Isolation Kit) Isolated Monocytes were resuspended at a density of 1×10⁶ cells/mL in culture media (RPMI 1640 containing 10% serum 1% pen/strep 2 mM L-glutamin) supplemented with 500 U/mL IL-4 and 500 U/mL GM-CSF. The cell culture was plated in 6-well tissue culture plates and incubated for 6 days. One day 3, half of fresh media supplemented with cytokines were added.

Animals 6 week C57BL/6 female mice were purchased from Jackson Laboratory and housed and treated according to the inventors' approved IACUC protocol.

Spleeyocyte Lymph Node Footpad extraction Spleeyocyte/Lymph Node/Footpad were dissected into 1 mm sized portions and placed in disassociation media (0.5 mg/mL collagenase D, 0.1 mg/mL DNAase I in RPMI) for 30 mins at room temp, then incubated at 37° C. for 30 mins, then passed through a 70 um filter. Footpads were incubated for 2 hrs rather than 30 mins. Spleenocytes were treated with RBC lysis buffer (Invitrogen) prior to final wash.

RAW Blue NF-κB Assay: RAW-Blue NF-κB cells (Invivogen) were passaged and plated in a 96 well plate at 100 k cells/well in 180 μL DMEM containing 10% HIFBS. Cells were incubated at 37° C. and 5% CO2 for 24 h. 100 ul of cells were incubated with varying ratios of MPs at 37° C. and 5% CO2 for 18 h. After 18 h, 20 μL of the cell supernatant was placed in 180 μL freshly prepared QuantiBlue (Invivogen) solution and incubated at 37° C./5% CO2 for up to 2 h. The plate was analyzed every hour using a Multiskan FC plate reader (Thermo Scientific) and absorbance was measured at 620 nm BCA Assay—This was performed according to manufacturer's instruction (Thermo Fischer) with some modifications. 100 million beads were incubated with BCA solution and reacted for 30 mins at 60° C. then analyzed every hour using a Multiskan FC plate reader (Thermo Scientific) and absorbance was measured at 562 nm and compared to a standard curve of modified MPLA or Pam2 after subtracting a background of maleimide modified MP.

Flow Cytometry Most flow cytometry in this study was performed on a ACEA NovaCyte Flow cytometer (6 channels, 2 laser). 1 million cells per sample were treated with liposomes/MPs/antibodies, washed and placed in HBSS+2% HIFBS+0.1 mM EDTA. Samples were gated on FCS and SCC for live and single cells. Samples were compensated based on sample with single stains and calculated using FlowJo.

Aurora SpectralFlow Analysis A 5 laser Aurora spectral flow cytometer was used for phenotyping DCs and T cells. Single stained compensation controls were run first and then unmixed using the Aurora spectral flow software. Data was further analyzed in FlowJo.

Image Stream MP Uptake Analysis for TNFa Expression: 1 million BMDCs, RAWs or Spleenocytess were incubated with 1 million MPs in 1 mL of cell culture media (DMEM supplanted with 10% HIFBS) for 1 hour. After 1 hr, protein export was inhibited using a GolgiPlug Kit (BD Biosciences) with Brefeldin A according to manufacturer's instructions for 16 hrs at 37° C. under 5% C02. Cells were then washed and fixed and permeabilized with a BD Cytofix/Cytoperm Plus Kit (BD Biosciences). Cells were stained with a solution of anti-TNFα (1:500 dilution) and their nuclei stained using Hoechst 33342 Solution (2 μM final concentration) in permeabilization buffer for 1 hr. Cells were washed 3× in PBS with 2% HIFBS, concentrated into a 20 μL volume and analyzed with ImageStream Flow Cytometry. Each MP condition was performed in triplicate, analyzing >100,000 cells per run. MPs were identified using the “Particle Count” wizard in the IDEAS software and compared to TNFα intensity per cell.

Image Stream MP Uptake Analysis for NF-κB Expression: 1 million BMDCs, RAWs or Spleenocytes were incubated with 1 million MPs in 1 mL of cell culture media (DMEM supplanted with 10% HIFBS) for 15 minutes. Immediately cells were fixed using ice cold Cytofixation Buffer (BD Biosciences) for 15 minutes. Cells were then fixed in PBS +0.04% triton X for 3 minutes, immediately spun down (400 RCF, 5 minutes), supernatant removed and washed with CytoPerm Buffer (BD Biosciences) 2 times (200 μL per wash). Cells were then stained with Rabbit anti-NF-κB p⁶⁵ (1:500 dilution) for 1 hr on ice, washed 3×, then stained with a secondary goat anti-rabbit AF647 (1:1000 dilution) for 1 hr on ice, washed, concentrated into a 20 μL volume of PBS with 2% HIFBS and analyzed with ImageStream Flow Cytometry. Each MP condition was performed in triplicate, analyzing >100,000 cells per run. MPs were identified using the “Particle Count” wizard in the IDEAS software and compared to NF-κB nuclear colocalization using the “Colocalization” wizard in the IDEAS software.

Imagestream Data Analysis: ImageStream data was first analyzed in the IDEAS software (Amnis) for nuclear colocalization and particle counting using built-in analysis wizards. Single Cell data was then exported into Graphpad Prism 6 software for further analysis. Cell data was divided into the following categories: 0 MP, 1 MP, 2 MP, 3 MP, 4 MP or 5 MP or >5 MPs.

Microscopy: BMDCs, Spleenocytes or RAWs were analyzed with a SP5 two photon confocal microscope. 100 k cells were allowed to attach to the bottom of a 96 well plate in their respective cell culture media. The next day, cells were washed with HBSS then either incubated with DiD containing liposomes for 15 mins, washed and incubated with MPs for 15 mins ord incubated with MPs for 15 mins, washed and then incubated with antibodies. Cells were washed and placed in fluorobrite media (Gibco) with 10% HIFBS+1:2000 dilution of Hoest then analyzed by microscopy using relevant wavelengths/filters. Single images were taken using a 60× lens.

CellSorting: BMDCs, Spleenocytes or RAWs were sorted on an Aria Fusion 5-18, ArialIIu 4-15 or AriaII 4-15 cell sorter. For sorting, cells were sometimes treated with or without Brefeldin A, then incubated with MPs for 15 mins, washed, scrapped, washed again and diluted to 20 million cells per mL in RPMI. Cells were gated on live and single cells by FSC and SCC and sorted into FR (top 5% of FITC signal from MPs) and nFR (bottom 90% of FITC signal from MPs). Cells were then immediately placed on ice, spun down at 4° C. and placed in cell culture media for subsequent experiments. For kinetic experiments, unsorted cell controls were also run through sorter but only gated on live and single cells.

Cytokine Bead Array a Mouse Inflammation CBA kit was purchased from BD Biosciences and used according to the manufacturer's instructions. Mouse blood was spun down at 10000 g for 10 mins to remove cells and the supernatant tested undiluted. Supernatant from cell culture experiments was also used with no dilution.

First Responder Cytokine Analysis: BMDCs were incubated at a 1:1 ratio with MP^{TLR-4} for 15 minutes and the FRs and nFRs were isolated via FACS. The cells were washed and resuspended at 1 million cells/mL in culture media (10% HIFBS in RPMI). The cells were incubated at 37° C. and 5% CO2 for 1 h. The supernatant was collected and stored at −80° C. until the cytokines were profiled using a mouse inflammation CBA kit (BD Biosciences) or via IL-10 Cytokine ELISA kit from Biolegend.

First Responder Kinetic Analysis: Sorted BMDC FRs or nFRs cells (BMDCs incubated 1:1 with MP-TLR4 for 15 mins, then sorted) were incubated at 100 k cells in 200 uL in cell culture media (10% HIFBS in RPMI) and incubated at 37° C. and 5% CO2 for varying time points. Then naïve BMDCs (1 million in 1 mL) were mixed with FRs or nFRs separated by a 1 um transwell insert or without for 16 hrs. Cell supernatants were tested via CBA for cytokine secretion and cells were tested via flow cytometry for CD80 (PerCP-Cy5.5 rat anti-mouse CD80, 1:100) and CD40 (APC rat anti-mouse CD40, 1:200), incubated for 1 hr at 4° C. then washed 3× with PBS, then tested via flow cytometry.

Adoptive Transfer of BMDCs: FRs and nFRs treated with MP-TLR4 similar to previous section were treated with DiL dye (1 ug/mL) and OVA (100 ug/mL) in 1 million cells in 1 mL cell culture media for 30 mins. Treated cells were then washed with PBS then concentrated at 1 million cells into 30 uL of HBSS and then immediately injected into C57BL/6 mouse footpads, one injection per footpad, two per mouse. 1 hr post injection, blood was taken for CBA analysis. 14 days later, mice were sacrificed, popliteal lymph nodes from both sides of the mouse were removed, disaggregated, stained and analyzed via Aurora spectral flow analysis.

mRNA Whole Transcriptome Analysis: For 16 h sequencing: BMDCs were incubated at a 1:1 ratio with MP^{TLR}-X (x=blank, 2, 4, 5, 7, 9, 2_4, 2_5, 2_7, 2_9, 4_5, 4_7, 4_9, 5_7, 5_9, 7_9) for 16 h. The FRs and nFRs were isolated via FACS. RNA was extracted using a Direct-zol RNA-Microprep kit (Zymo), prepped using SMARTer® Stranded Total RNASeq Kit v2 (Takara), and sequenced on a NextSeq550 (Illumina). RNA seq reads were mapped to GRCm38 mouse reference genome using STAR version 2.7.0b⁶ The resulting files from the alignment step above were taken to evaluate transcriptional expression using subread:featureCounts with gencode transcript annotation M19.⁷ The obtained count table was normalized and log fold change in expression was generated using the edgeR package⁸. Using the Cell Surface Protein Atlas's database or mouse cell surface protein, the inventors identified proteins that were most frequently upregulated in the most MP^{TLR-X} dosing conditions that met both the following criteria: 2-fold upregulation and pval <0.05.9 For time series sequencing: BMDCs were incubated at 1:1 ratio with MP^{TLR-4} for 15 min. The FRs and nFRs were isolated via FACS, washed, and resuspended in media. At 0, 0.5, 1, 2, 4 h, the RNA was extracted using a Direct-zol RNA-Microprep kit (Zymo). Sequencing was performed by the University of Chicago Genomics Core, and BasePairTech's DESEQ2 pipeline was used to align the reads to the mm10 genome, and compute the differential expression and GSEA analysis. Using the Mouse Genomic Informatics database to acquire lists of genes with 1) immune function 2) antigen presentation the immune response of the FRs and nFRs were profiled by analyzing the differential expression, using only genes with 2-fold differential expression change and pval <0.05 for at least one of the timepoints.¹⁰

Anti-OVA ELISA: Mouse anti-OVA IgG were measured using a commercially available kit from Alpha Diagnostic International according to the manufacturer's instructions.

Brefeldin A In vivo Experiment: 6 week, C57BL/6 female mice, 5 mice per experimental group were injected with liposomes containing brefeldin A or free Brefeldin A or PBS control. Mice were injected with 100 ug of Brefeldin A (either free or loaded equivalent) i.p. and then 1 hr later mice were injected i.p. with 10 ug R848 and 100 ug OVA in PBS. 14 days later this injection procedure was repeated. On day 21, mice were sac'd, blood tested for anti-OVA IgG and lymph and spleen analyzed.

GpG Loaded Liposome In vivo: Experiment 6 week, C57BL/6 female mice, 5 mice per experimental group were injected with liposomes containing CpG or free CpG or PBS control. Mice were injected with 10 ug of CpG (either free or loaded equivalent) with 100 ug OVA i.p. On day 14, mice were sac'd, blood tested for anti-OVA IgG and lymph and spleen analyzed.

R848 Loaded Liposome In vivo Experiment: 6 week, C57BL/6 female mice, 5 mice per experimental group were injected with liposomes containing R848 or free R848 or PBS control. Mice were injected with 10 ug of R848 (either free or loaded equivalent) with 100 ug OVA i.p. On day 14, this procedure was repeated. On day 21, mice were sac'd, blood tested for anti-OVA IgG and lymph and spleen analyzed.

Intracellular Staining: Fresh suspensions of mouse spleenocytes were incubated with OVA peptides for 30 mins in ICS media (RPMI, 10% FBS, Penicillin/Streptomycin, 1× Non-Essential Amino Acids, 1 um B-mercaptoethanol, 1 mM HEPES and 1 mM Sodium Pyruvate. Then spleenocytes were incubated for 12 hrs with monensin (1 ug/mL), washed, fixed and stained for intracellular and extracellular antigens (CD4, CD8, INFγ and TL-4) according to the manufacture's instruction for BD Cytofix/Cytoperm Kit.

E. G7 OVA Tumor Model: C57BL/6 female mice were injected with 5×10⁵ E. G7 OVA expressing cells in PBS in the left flank. Mice were shaved to observe tumor growth. On day 7 and day 10, mice were injected with liposome formulation or free R848/OVA (10 ug/100 ug per mouse) i.p. Tumor volume was tracked via calipers 3 times per week and volume calculated by the formula Volume=(½) (length*(width)\hat{ }2).¹¹

Statistics: Unless otherwise noted, all statistics were performed using a student's t test with significance p<0.05. Poission distribution was used to calculate the standard distribution of discrete particles where Probability of Number of MP uptaken=P, κ=Number

$$P = \frac{{λ}^{κ} {e}^{- λ}}{k !}$$

### Example 2—Additional Characterization and Targeting of First Responder Dendritic Cells

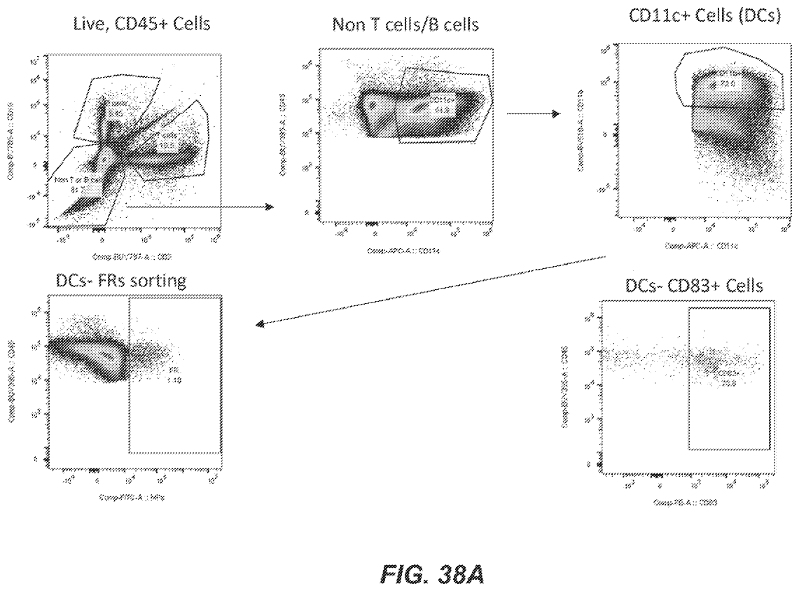

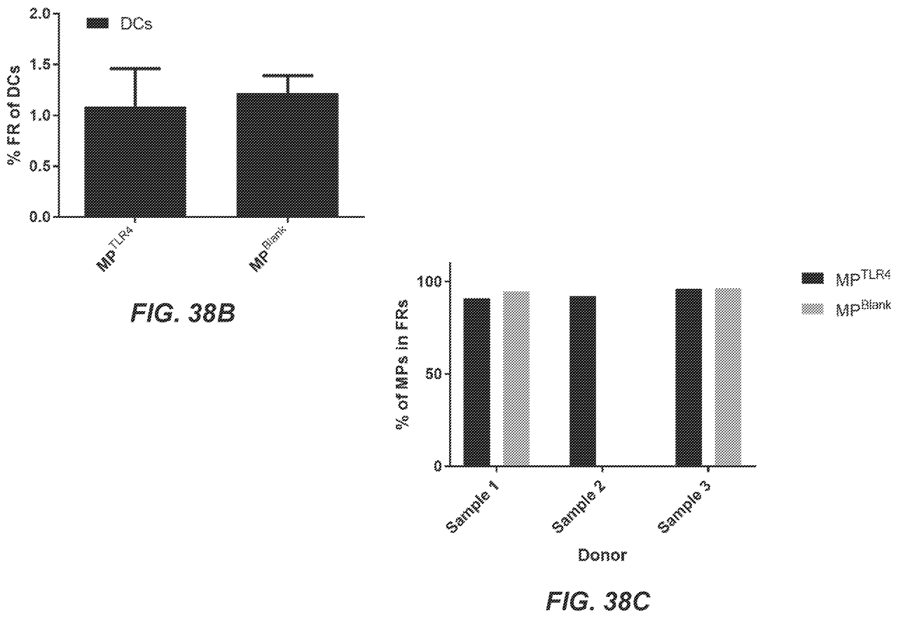

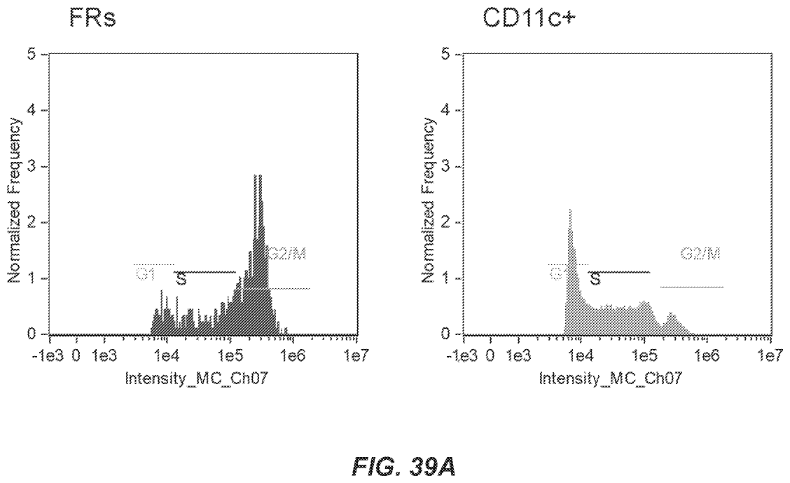

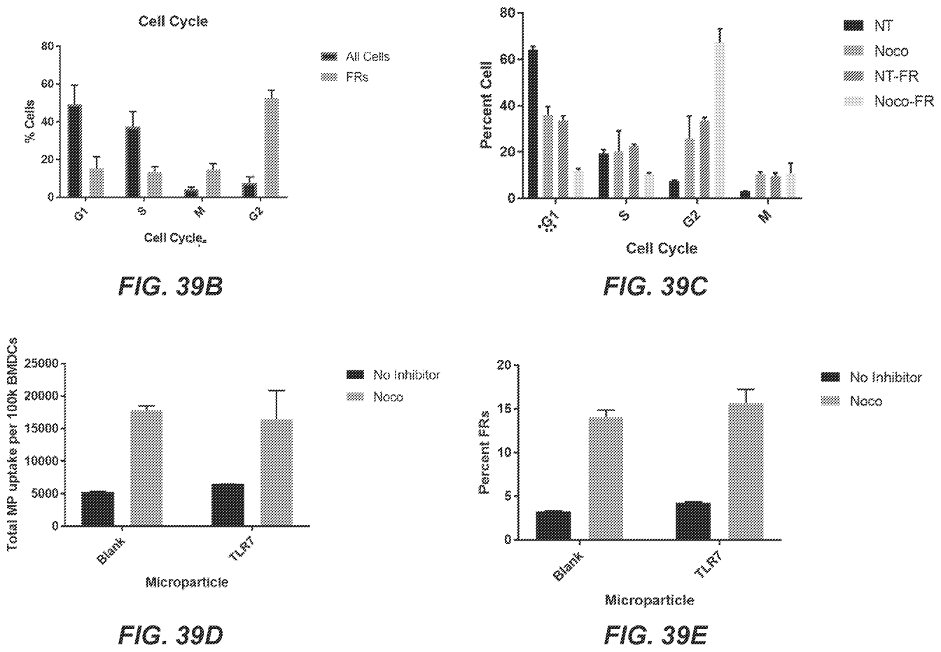

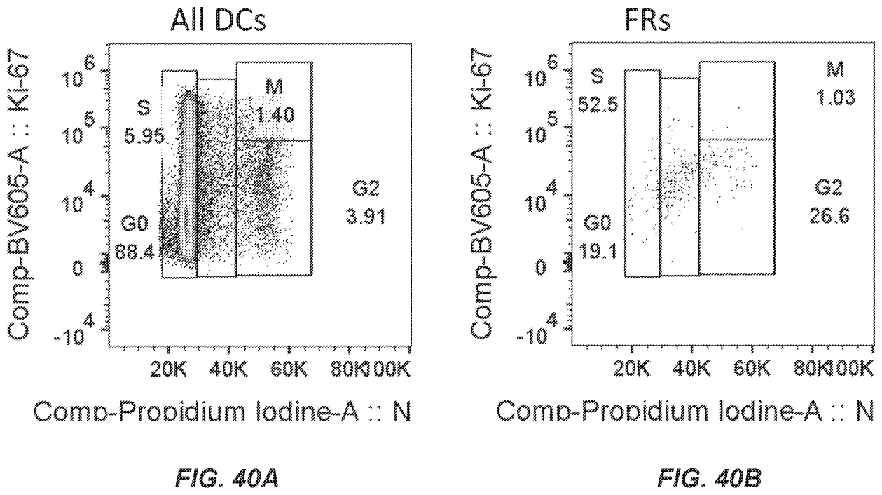

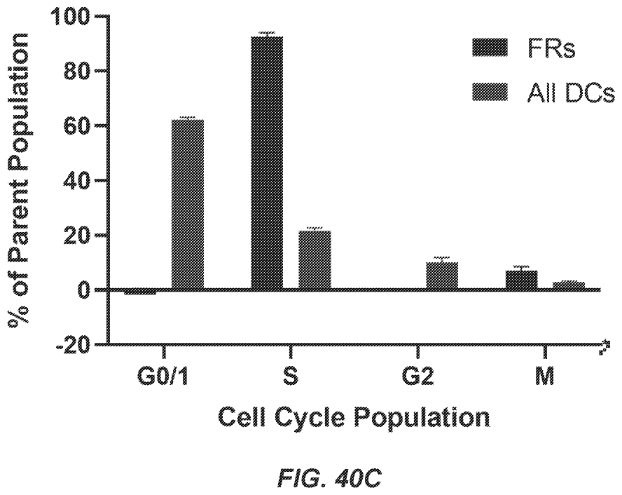

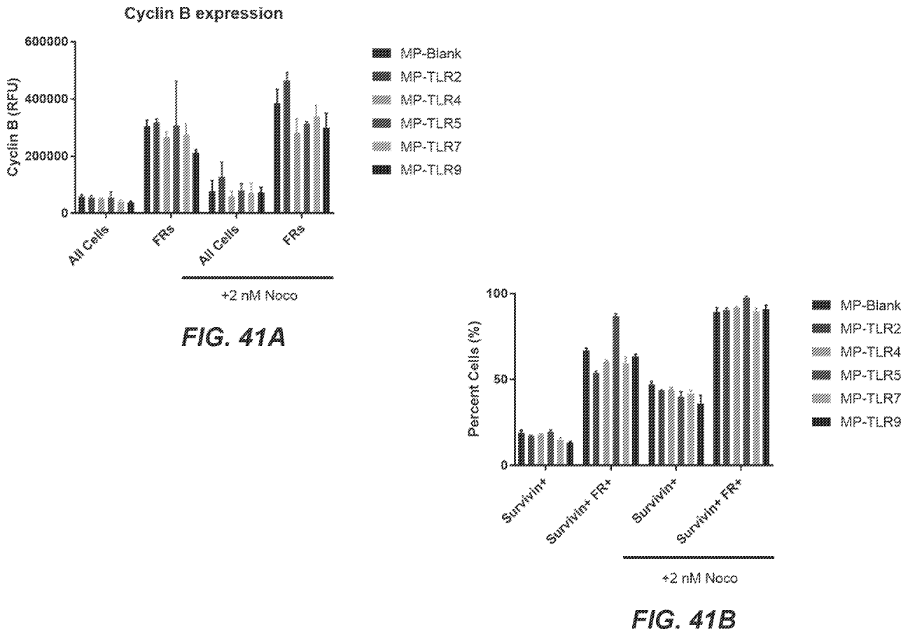

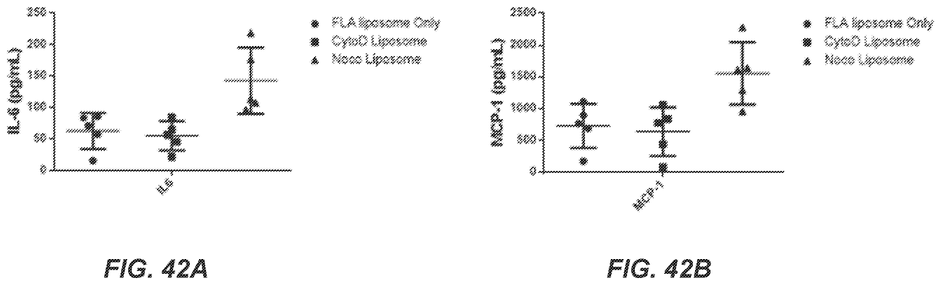

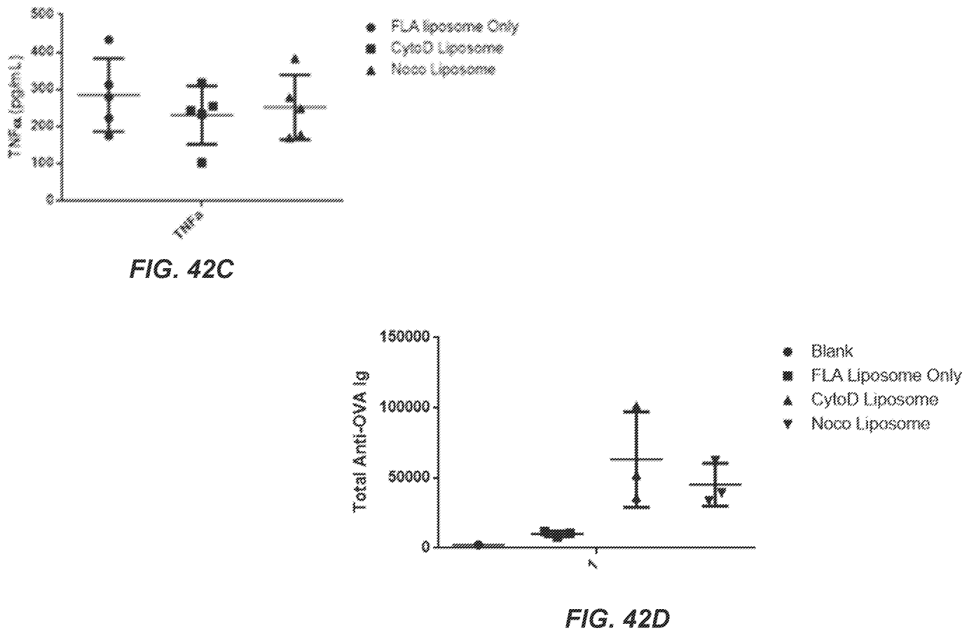

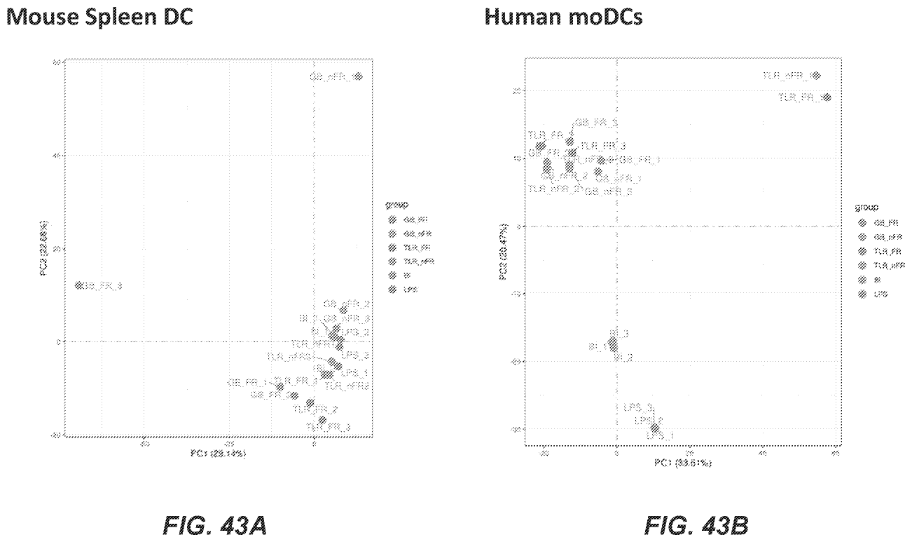

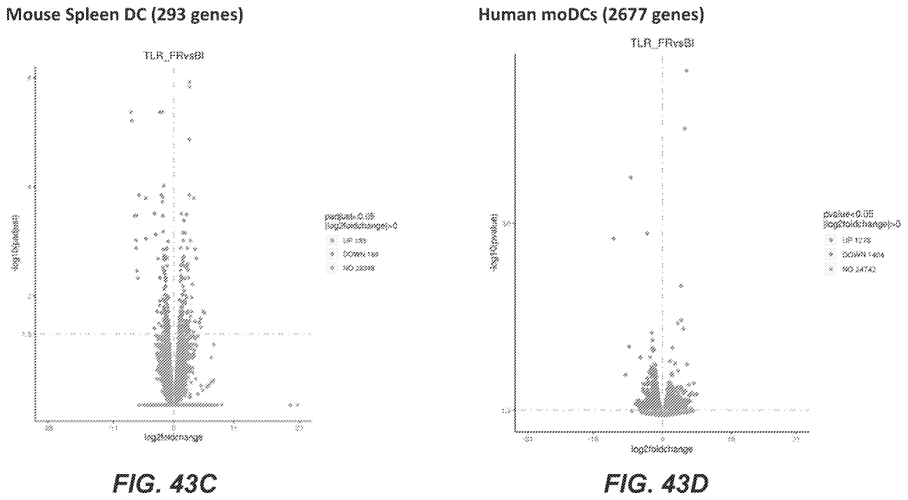

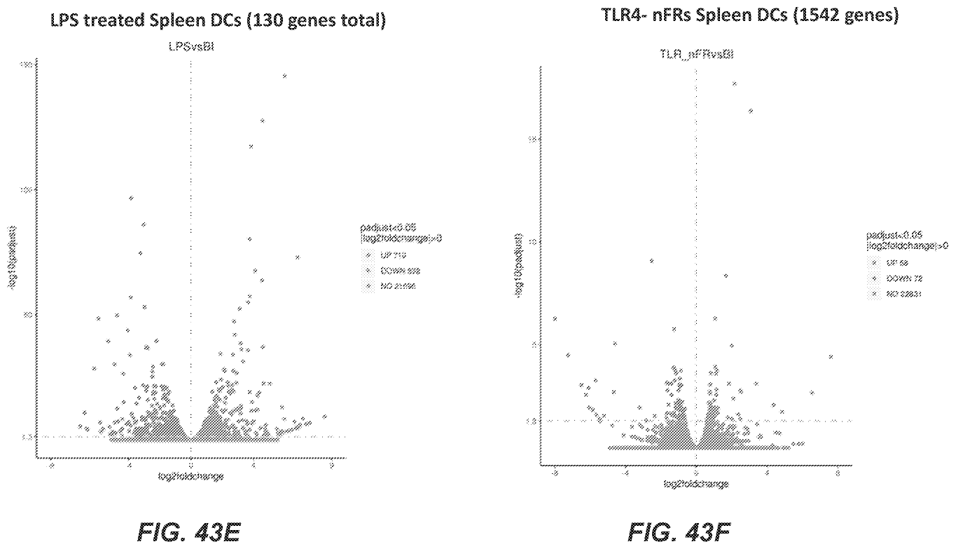

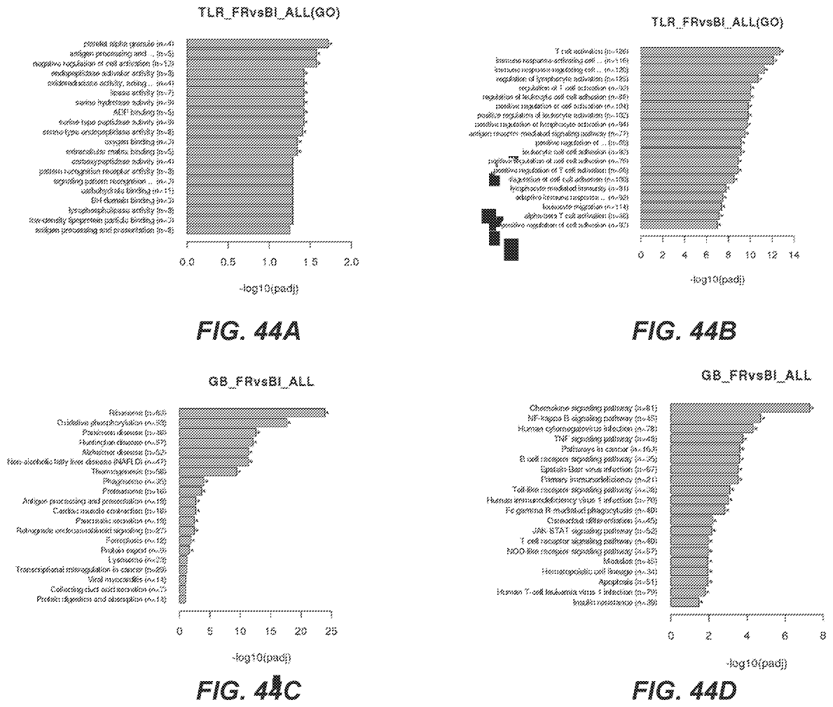

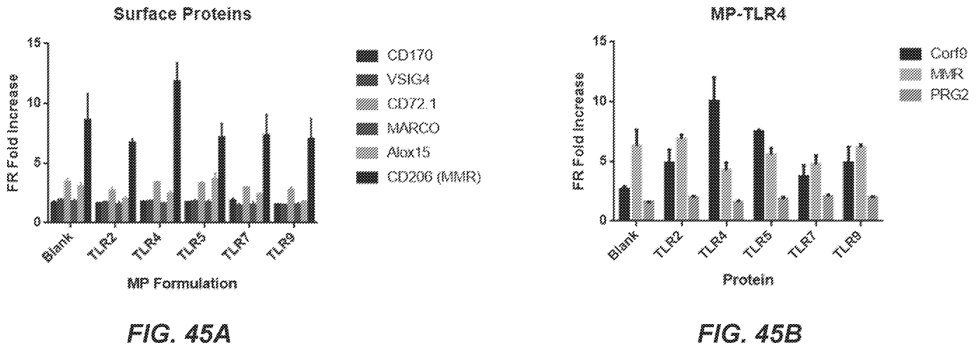

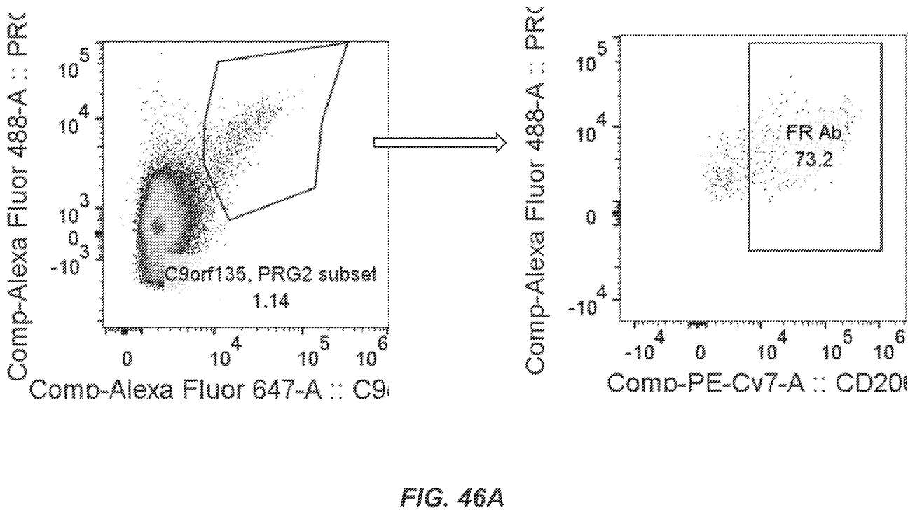

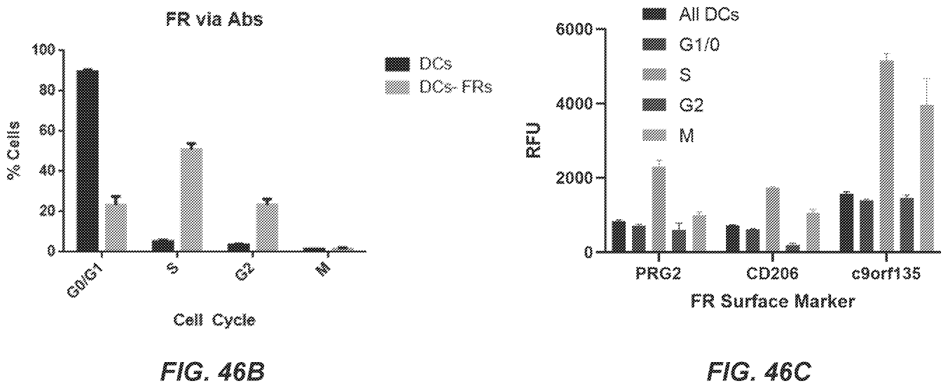

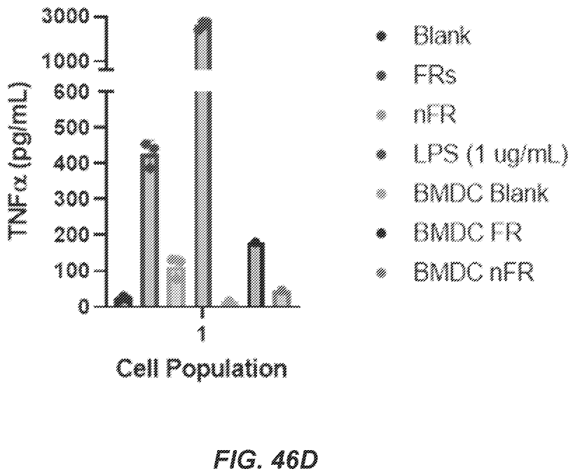

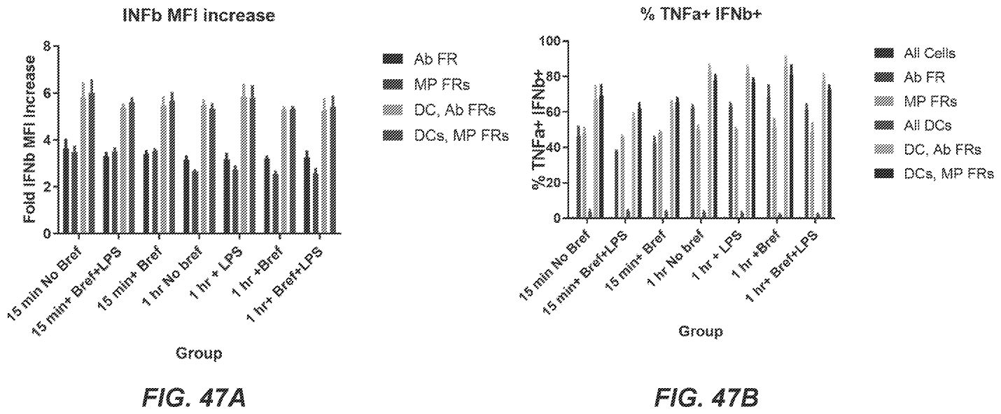

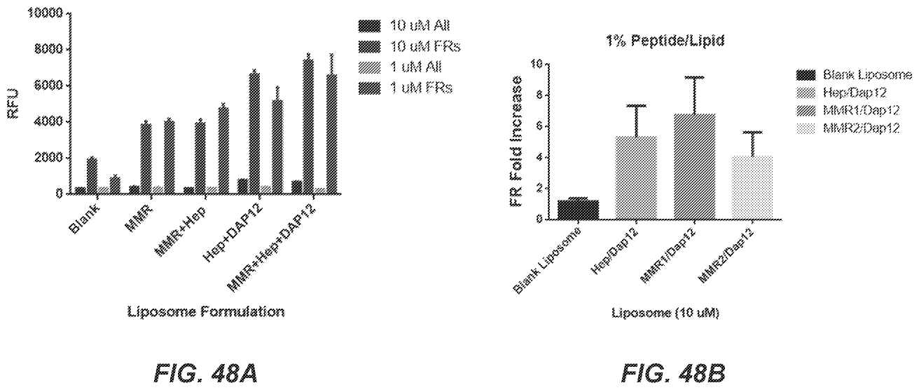

Additional characterization and targeting of first responder dendritic cells (FRs) was performed. Results from these studies are shown in FIGS. 38A-48B. These studies demonstrate that FRs exist in human dendritic cell populations, that FRs are more likely actively dividing, that increasing G2 phase in vivo (e.g., using an agent that blocks cells in G1 phase such as Cytoclastin D) increases vaccine response, that FRs express unique surface proteins including those shown in Table 3, that FRs can be isolated via antibodies against PRG2, CD206 and C9orfl35, and that a peptide for CD206 targeting having sequence RWKFGGFKWR (SEQ ID NO:2), in combination with heparin for PRG2 targeting or the polypeptide having sequence GFLSKSLVF (SEQ ID NO:1) for DAP12 targeting, can identify and target FRs.

TABLE 3
FR related proteins

| | TLR4_FR/Blank | | TLR4_FR/TLR4_nFR | |
|---|---|---|---|---|
| Protein | log2foldchange | pval | log2foldchange | pval |
| CD37 | 1.8 | 4.2E−05 | 0.7 | 1.5E−02 |
| CD79a | 2.0 | 2.2E−02 | 1.0 | 3.4E−02 |
| SIGLEC5 | 1.6 | 1.2E−01 | 1.0 | 6.5E−02 |
| VSIG4 | 7.3 | 5.7E−09 | 1.7 | 9.1E−02 |
| CD72.1 | 2.0 | 2.6E−03 | 0.2 | 8.0E−02 |
| FCGR3A | 7.4 | 5.2E−06 | 1.0 | 1.1E−01 |
| (CD16) | | | | |
| C9orf135 | 6.8 | 1.6E−06 | 7.4 | 5.2E−06 |
| CXCR1 | 1.1 | 2.4E−02 | 3.4 | 1.8E−01 |
| HLA-DQA1 | 3.9 | 3.8E−02 | 0.8 | 5.5E−02 |
| CD206 (MMR) | 1.6 | 7.9E−05 | 3.1 | 7.9E−05 |

Table 3 shows FR related proteins having a significant foldchange in mouse splenocytes stimulated with LPS conjugated microparticles, either treated/untreated cells (Blank) or treated FR cells/treated non-FR (nFR) cells.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
(1) Brown, C. C.; Gudjonson, H.; Pritykin, Y.; Deep, D.; Lavallee, V.-P.; Mendoza, A.; Fromme, R.; Mazutis, L.; Ariyan, C.; Leslie, C.; Pe'er, D.; Rudensky, A. Y. Transcriptional Basis of Mouse and Human Dendritic Cell Heterogeneity. Cell 2019, 179 (4), 846-863.e24. https://doi.org/10.1016/j.cell.2019.09.035.
(2) Xue, Q.; Lu, Y.; Eisele, M. R.; Sulistijo, E. S.; Khan, N.; Fan, R.; Miller-Jensen, K. Analysis of Single-Cell Cytokine Secretion Reveals a Role for Paracrine Signaling in Coordinating Macrophage Responses to TLR4 Stimulation. Sci. Signal. 2015, 8 (381), ra59-ra59. https://doi.org/10.1126/scisignal.aaa2155.
(3) Moser, B. A.; Steinhardt, R. C.; Esser-Kahn, A. P. Surface Coating of Nanoparticles Reduces Background Inflammatory Activity While Increasing Particle Uptake and Delivery. ACS Biomater Sci Eng 2017, 3 (2), 206-213. https://doi.org/10.1021/acsbiomaterials.6b00473.
(4) Schuijs, M. J.; Hammad, H.; Lambrecht, B. N. Professional and “Amateur” Antigen-Presenting Cells In Type 2 Immunity. Trends Immunol. 2019, 40 (1), 22-34. https://doi.org/10.1016/j.it.2018.11.001.
(5) Banchereau, J.; Steinman, R. M. Dendritic Cells and the Control of Immunity. Nature 1998, 392 (6673), 245-252. https://doi.org/10.1038/32588.
(6) Tuana, G.; Volpato, V.; Ricciardi-Castagnoli, P.; Zolezzi, F.; Stella, F.; Foti, M. Classification of Dendritic Cell Phenotypes from Gene Expression Data. BMC Immunology 2011, 12 (1), 50. https://doi.org/10.1186/1471-2172-12-50.
(7) Krutzik, S. R.; Tan, B.; Li, H.; Ochoa, M. T.; Liu, P. T.; Sharfstein, S. E.; Graeber, T. G.; Sieling, P. A.; Liu, Y.-J.; Rea, T. H.; Bloom, B. R.; Modlin, R. L. TLR Activation Triggers the Rapid Differentiation of Monocytes into Macrophages and Dendritic Cells. Nature Medicine 2005, 11 (6), 653-660. https://doi.org/10.1038/nm1246.
(8) Shalek, A. K.; Satija, R.; Shuga, J.; Trombetta, J. J.; Gennert, D.; Lu, D.; Chen, P.; Gertner, R. S.; Gaublomme, J. T.; Yosef, N.; Schwartz, S.; Fowler, B.; Weaver, S.; Wang, J.; Wang, X.; Ding, R.; Raychowdhury, R.; Friedman, N.; Hacohen, N.; Park, H.; May, A. P.; Regev, A. Single Cell RNA Seq Reveals Dynamic Paracrine Control of Cellular Variation. Nature 2014, 510 (7505), 363-369. https://doi.org/10.1038/naturel3437.
(9) Long, M.; Adler, A. J. Cutting Edge: Paracrine, but Not Autocrine, IL-2 Signaling Is Sustained during Early Antiviral CD4 T Cell Response. J Immunol 2006, 177 (7), 4257-4261.
(10) Filep, J. G.; Ariel, A. Neutrophil Heterogeneity and Fate in Inflamed Tissues: Implications for the Resolution of Inflammation. American Journal of Physiology-Cell Physiology 2020, 319 (3), C510-C532. https://doi.org/10.1152/ajpcell.00181.2020.
(11) Wimmers, F.; Subedi, N.; van Buuringen, N.; Heister, D.; Vivie, J.; Beeren-Reinieren, I.; Woestenenk, R.; Dolstra, H.; Piruska, A.; Jacobs, J. F. M.; van Oudenaarden, A.; Figdor, C. G.; Huck, W. T. S.; de Vries, I. J. M.; Tel, J. Single-Cell Analysis Reveals That Stochasticity and Paracrine Signaling Control Interferon-Alpha Production by Plasmacytoid Dendritic Cells. Nature Communications 2018, 9 (1), 1-12. https://doi.org/10.1038/s41467-018-05784-3.
(12) Deak, P.; Kimani, F.; Cassaidy, B.; Esser-Kahn, A. Determining Whether Agonist Density or Agonist Number Is More Important for Immune Activation via Micoparticle Based Assay. Front. Immunol. 2020, 11. https://doi.org/10.3389/fimmu.2020.00642.
(13) Pacheco, P.; White, D.; Sulchek, T. Effects of Microparticle Size and Fc Density on Macrophage Phagocytosis. PLOS ONE 2013, 8 (4), e60989. https://doi.org/10.1371/journal.pone.0060989.
(14) Meraz, I. M.; Melendez, B.; Gu, J.; Wong, S. T. C.; Liu, X.; Andersson, H. A.; Serda, R. E. Activation of the Inflammasome and Enhanced Migration of Microparticle-Stimulated Dendritic Cells to the Draining Lymph Node. Mol. Pharmaceutics 2012, 9 (7), 2049-2062. https://doi.org/10.1021/mp3001292.
(15) Chometon, T. Q.; Siqueira, M. da S.; Sant'anna, J. C.; Almeida, M. R.; Gandini, M.; Nogueira, A. C. M. de A.; Antas, P. R. Z. A Protocol for Rapid Monocyte Isolation and Generation of Singular Human Monocyte-Derived Dendritic Cells. PLOS ONE 2020, 15 (4), e0231132. https://doi.org/10.1371/joumal.pone.0231132.
(16) Sedger, L. M.; McDermett, M. F. TNF and TNF-Receptors: From Mediators of Cell Death and Inflammation to Therapeutic Giants—Past, Present and Future. Cytokine & Growth Factor Rev. 2014, 25 (4), 453-472. https://doi.org/10.1016/j.cytogfr.2014.07.016.
(17) Elgetta, R.; Benson, M. J.; de Vries, V. C.; Wasluk, A.; Guo, Y.; Noelle, R. J. Molecular Mechanism and Function of CD40/CD40L Engagement in the Immune System. Immunol Rev. 2009, 229 (1). https://dx.doi.org/10.1111%2Fj.1600-065X.2009.00782.x.
(18) Gao, F. G.; Khammanivong, V.; Liu, W. J.; Leggatt, G. R.; Frazer, I. H.; Fernando, G. J. P. Antigen-Specific CD4+ T-Cell Help Is Required to Activate a Memory CD8+ T Cell to a Fully Functional Tumor Killer Cell. Cancer Res 2002, 62 (22), 6438-6441.
(19) Dearman, R. J.; Cumberbatch, M.; Maxwell, G.; Basketter, D. A.; Kimber, I. Toll-like Receptor Ligand Activation of Murine Bone Marrow-Derived Dendritic Cells. Immunology 2009, 126 (4), 475-484. https://doi.org/10.1111/j.1365-2567.2008.02922.x.
(20) Liu, Y.; Beyer, A.; Aebersold, R. On the Dependency of Cellular Protein Levels on MRNA Abundance. Cell 2016, 165 (3), 535-550. https://doi.org/10.1016/j.cell.2016.03.014.
(21) Price, B. D.; Mannheim-Rodman, L. A.; Calderwood, S. K. Brefeldin A, Thapsigargin, and AlF Stimulate the Accumulation of GRP78 MRNA in a Cycloheximide Dependent Manner, Whilst Induction by Hypoxia Is Independent of Protein Synthesis. Journal of Cellular Physiology 1992, 152 (3), 545-552. https://doi.org/10.1002/jcp.1041520314.
(22) Ogasawara, H.; Furuno, M.; Edamura, K.; Noguchi, M. Peptides of Major Basic Protein and Eosinophil Cationic Protein Activate Human Mast Cells. Biochem Biophys Rep 2020, 21, 100719. https://doi.org/10.1016/j.bbrep.2019.100719.
(23) Bouchon, A.; Hernindez-Munain, C.; Cella, M.; Colonna, M. A Dapl2-Mediated Pathway Regulates Expression of Cc Chemokine Receptor 7 and Maturation of Human Dendritic Cells. J Exp Med 2001, 194 (8), 1111-1122. https://doi.org/10.1084/jem.194.8.1111.
(24) Picotto, G.; Morse, L. R.; Nguyen, N.; Saltzman, J.; Battaglino, R. TMEM176A and TMEM176B Are Candidate Regulators of Inhibition of Dendritic Cell Maturation and Function after Chronic Spinal Cord Injury. Journal of Neurotrauma 2019, 37 (3), 528-533. https://doi.org/10.1089/neu.2019.6498.
(25) Shen, Z. T.; Sigalov, A. B. Rationally Designed Ligand-Independent Peptide Inhibitors of TREM-1 Ameliorate Collagen-Induced Arthritis. Journal of Cellular and Molecular Medicine 2017, 21 (10), 2524-2534. https://doi.org/10.1111/jcmm.13173.
(26) Weiler, J. M.; Gleich, G. J. Eosinophil Granule Major Basic Protein Regulates Generation of Classical and Alternative-Amplification Pathway C3 Convertases in Vitro. The Journal of Immunology 1988, 140 (5), 1605-1610.
(27) Kim, E.; Kim, J. C.; Min, K.; Goh, M.; Tae, G. Rapid and Versatile Cell Aggregate Formation Using Lipid-Conjugated Heparin. ACS Applied Materials & Interfaces 2018. https://doi.org/10.1021/acsami.8b07731.
(28) Stefanick, J. F.; Ashley, J. D.; Kiziltepe, T.; Bilgicer, B. A Systematic Analysis of Peptide Linker Length and Liposomal Polyethylene Glycol Coating on Cellular Uptake of Peptide-Targeted Liposomes. ACS Nano 2013, 7 (4), 2935-2947. https://doi.org/10.1021/nn305663e.
(29) Gursel, I.; Gursel, M.; Ishii, K. J.; Klinman, D. M. Sterically Stabilized Cationic Liposomes Improve the Uptake and Immunostimulatory Activity of CpG Oligonucleotides. The Journal of Immunology 2001, 167 (6), 3324-3328. https://doi.org/10.4049/jimmunol.167.6.3324.
(30) Rodell, C. B.; Arlauckas, S. P.; Cuccarese, M. F.; Garris, C. S.; Li, R.; Ahmed, M. S.; Kohler, R. H.; Pittet, M. J.; Weissleder, R. TLR7/8-Agonist-Loaded Nanoparticles Promote the Polarization of Tumour-Associated Macrophages to Enhance Cancer Immunotherapy. Nat Biomed Eng 2018, 2, 578-588.
(31) Predina, J.; Eruslanov, E.; Judy, B.; Kapoor, V.; Cheng, G.; Wang, L.-C.; Sun, J.; Moon, E. K.; Fridlender, Z. G.; Albelda, S.; Singhal, S. Changes in the Local Tumor Microenvironment in Recurrent Cancers May Explain the Failure of Vaccines after Surgery. PNAS 2013, 110 (5), E415-E424. https://doi.org/10.1073/pnas.1211850110.
(32) Korhan, P.; Yilmaz, Y.; Bağirsakçi, E.; Güneş, A.; Topel, H.; Carr, B. I.; Atabey, N. Pleiotropic Effects of Heparins: From Clinical Applications to Molecular Mechanisms in Hepatocellular Carcinoma. Can J Gastroenterol Hepatol 2018, 2018. https://doi.org/10.1155/2018/7568742.
(33) Molgora, M.; Esaulova, E.; Vermi, W.; Hou, J.; Chen, Y.; Luo, J.; Brioschi, S.; Bugatti, M.; Omodei, A. S.; Ricci, B.; Fronick, C.; Panda, S. K.; Takeuchi, Y.; Gubin, M. M.; Faccio, R.; Cella, M.; Gilfillan, S.; Unanue, E. R.; Artyomov, M. N.; Schreiber, R. D.; Colonna, M. TREM2 Modulation Remodels the Tumor Myeloid Landscape Enhancing Anti-PD-1 Immunotherapy. Cell 2020, 182 (4), 886-900.e17. https://doi.org/10.1016/j.cell.2020.07.013.
(34) Obregon, C.; Rothen-Rutishauser, B.; Gerber, P.; Gehr, P.; Nicod, L. P. Active Uptake of Dendritic Cell-Derived Exovesicles by Epithelial Cells Induces the Release of Inflammatory Mediators through a TNF-α-Mediated Pathway. Am J Pathol 2009, 175 (2), 696-705. https://doi.org/10.2353/ajpath.2009.080716.
(35) Pearce, E. L.; Pearce, E. J. Metabolic Pathways In Immune Cell Activation And Quiescence. Immunity 2013, 38 (4), 633-643. https://doi.org/10.1016/j.immuni.2013.04.005.
(36) Johansson, U.; Walther-Jallow, L.; Smed-Sorensen, A.; Spetz, A.-L. Triggering of Dendritic Cell Responses after Exposure to Activated, but Not Resting, Apoptotic PBMCs. The Journal of Immunology 2007, 179 (3), 1711-1720. https://doi.org/10.4049/jimmunol.179.3.1711.

1. A pharmaceutical composition comprising:
(a) an antigen or a polynucleotide encoding an antigen; and
(b) a first responder (FR)-targeting agent.

2. The pharmaceutical composition of claim 1, wherein the FR-targeting agent is conjugated to the antigen.

3. The pharmaceutical composition of claim 1, wherein the FR-targeting agent is conjugated to a liposome comprising the antigen or polynucleotide encoding the antigen.

4. The pharmaceutical composition of claim 1, wherein the FR-targeting agent is conjugated to a nanoparticle comprising the antigen or polynucleotide encoding the antigen.

5. The pharmaceutical composition of any of claims 1-4, further comprising an adjuvant.

6. The pharmaceutical composition of claim 5, wherein the adjuvant is a TLR agonist.

7. The pharmaceutical composition of claim 6, wherein the TLR agonist is a TLR9 agonist.

8. The pharmaceutical composition of claim 7, wherein the TLR9 agonist is a CpG oligodeoxynucleotide (ODN).

9. The pharmaceutical composition of claim 6, wherein the TLR agonist is a TLR7 agonist.

10. The pharmaceutical composition of claim 9, wherein the TLR7 agonist is R848.

11. The pharmaceutical composition of any of claims 1-10, wherein the FR-targeting agent is an agent capable of binding to a protein of Table 1.

12. The pharmaceutical composition of any of claims 1-11, wherein the FR-targeting agent is a PRG2-binding agent.

13. The pharmaceutical composition of claim 12, wherein the PRG2-binding agent is heparin.

14. The pharmaceutical composition of any of claims 1-11, wherein the FR-targeting agent is a DAP12-binding agent.

15. The pharmaceutical composition of claim 14, wherein the DAP12-binding agent is a polypeptide comprising SEQ ID NO:1.

16. The pharmaceutical composition of any of claims 1-11, wherein the FR-targeting agent is a CD206-targeting agent.

17. The pharmaceutical composition of claim 16, wherein the CD206-targeting agent is a polypeptide comprising SEQ ID NO:2.

18. The pharmaceutical composition of any of claims 1-11, wherein the FR-targeting agent is a C9orfl35-targeting agent.

19. The pharmaceutical composition of any of claims 1-11, wherein the FR-targeting agent is a TMEM176A-binding agent.

20. The pharmaceutical composition of any of claims 1-11, wherein the FR-targeting agent is a TREM2-binding agent.

21. The pharmaceutical composition of any of claims 1-11, wherein the FR-targeting agent is a CLC5A-binding agent.

22. The pharmaceutical composition of any of claims 1-21, further comprising an additional FR-targeting agent.

23. The pharmaceutical composition of claim 22, wherein the FR-targeting agent is a PRG2-binding agent and the additional FR-targeting agent is a DAP12-binding agent.

24. The pharmaceutical composition of claim 23, wherein the PRG2-binding agent is heparin and the DAP12-binding agent is a polypeptide comprising SEQ ID NO:1.

25. The pharmaceutical composition of claim 22, wherein the FR-targeting agent is a PRG2-binding agent and the additional FR-targeting agent is a CD206-binding agent.

26. The pharmaceutical composition of claim 25, wherein the PRG2-binding agent is heparin and the CD206-binding agent is a polypeptide comprising SEQ ID NO:2.

27. The pharmaceutical composition of claim 22, wherein the FR-targeting agent is a DAP12-binding agent and the additional FR-targeting agent is a CD206-binding agent.

28. The pharmaceutical composition of claim 27, wherein the DAP12-binding agent is a polypeptide comprising SEQ ID NO:1 and the CD206-binding agent is a polypeptide comprising SEQ ID NO:2.

29. The pharmaceutical composition of any of claims 1-28, wherein the antigen is a tumor antigen.

30. The pharmaceutical composition of any of claims 1-28, wherein the antigen is a viral antigen.

31. The pharmaceutical composition of any of claims 1-28, wherein the antigen is a bacterial antigen.

32. A method for stimulating an immune response to an antigen comprising administering to a subject an effective amount of the pharmaceutical composition of any of claims 1-31.

33. The method of claim 32, wherein the subject is a mouse subject.

34. The method of claim 32, wherein the subject is a human subject.

35. A method for stimulating an immune response to an antigen comprising administering to a subject an effective amount of a pharmaceutical composition comprising an antigen and a First Responder (FR)-targeting agent.

36. The method of claim 35, wherein the FR-targeting agent is conjugated to the antigen.

37. The method of claim 35, wherein the FR-targeting agent is conjugated to a liposome comprising the antigen or polynucleotide encoding the antigen.

38. The method of claim 35, wherein the FR-targeting agent is conjugated to a nanoparticle comprising the antigen or polynucleotide encoding the antigen.

39. The method of any of claims 35-38, further comprising an adjuvant.

40. The method of claim 39, wherein the adjuvant is a TLR agonist.

41. The method of claim 40, wherein the TLR agonist is a TLR9 agonist.

42. The method of claim 41, wherein the TLR9 agonist is a CpG ODN.

43. The method of claim 40, wherein the TLR agonist is a TLR7 agonist.

44. The method of claim 43, wherein the TLR7 agonist is R848.

45. The method of any of claims 35-44, wherein the FR-targeting agent is an agent capable of binding to a protein of Table 1.

46. The method of any of claims 35-45, wherein the FR-targeting agent is a PRG2-binding agent.

47. The method of claim 46, wherein the PRG2-binding agent is heparin.

48. The method of any of claims 35-45, wherein the FR-targeting agent is a DAP12-binding agent.

49. The method of claim 48, wherein the DAP12-binding agent is a polypeptide comprising SEQ ID NO:1.

50. The method of any of claims 35-45, wherein the FR-targeting agent is a CD206-targeting agent.

51. The method of claim 50, wherein the CD206-targeting agent is a polypeptide comprising SEQ ID NO:2.

52. The method of any of claims 35-45, wherein the FR-targeting agent is a C9orfl35-targeting agent.

53. The method of any of claims 35-45, wherein the FR-targeting agent is a TMEM176A-binding agent.

54. The method of any of claims 35-45, wherein the FR-targeting agent is a TREM2-binding agent.

55. The method of any of claims 35-45, wherein the FR-targeting agent is a CLC5A-binding agent.

56. The method of any of claims 35-55, further comprising an additional FR-targeting agent.

57. The method of claim 56, wherein the FR-targeting agent is a PRG2-binding agent and the additional FR-targeting agent is a DAP12-binding agent.

58. The method of claim 57, wherein the PRG2-binding agent is heparin and the DAP12-binding agent is a polypeptide comprising SEQ ID NO:1.

59. The method of claim 57, wherein the FR-targeting agent is a PRG2-binding agent and the additional FR-targeting agent is a CD206-binding agent.

60. The method of claim 59, wherein the PRG2-binding agent is heparin and the CD206-binding agent is a polypeptide comprising SEQ ID NO:2.

61. The method of claim 57, wherein the FR-targeting agent is a DAP12-binding agent and the additional FR-targeting agent is a CD206-binding agent.

62. The method of claim 61, wherein the DAP12-binding agent is a polypeptide comprising SEQ ID NO:1 and the CD206-binding agent is a polypeptide comprising SEQ ID NO:2.

63. The method of any of claims 35-62, wherein the antigen is a tumor antigen.

64. The method of any of claims 35-62, wherein the antigen is a viral antigen.

65. The method of any of claims 35-62, wherein the antigen is a bacterial antigen.

66. The method of any of claims 35-65, wherein the subject is a mouse subject.

67. The method of any of claims 35-65, wherein the subject is a human subject.

68. A method for treating or preventing cancer in a subject comprising administering to the subject an effective amount of a pharmaceutical composition comprising a tumor antigen and a First Responder (FR)-targeting agent.

69. The method of claim 68, further comprising administering to the subject an additional cancer therapy.

70. The method of claim 69, wherein the additional cancer therapy is a chemotherapy, radiotherapy, or immunotherapy.

71. The method of any of claims 68-70, wherein the FR-targeting agent is conjugated to the antigen.

72. The method of any of claims 68-70, wherein the FR-targeting agent is conjugated to a liposome comprising the antigen or polynucleotide encoding the antigen.

73. The method of any of claims 68-70, wherein the FR-targeting agent is conjugated to a nanoparticle comprising the antigen or polynucleotide encoding the antigen.

74. The method of any of claims 68-73, further comprising an adjuvant.

75. The method of claim 74, wherein the adjuvant is a TLR agonist.

76. The method of claim 75, wherein the TLR agonist is a TLR9 agonist.

77. The method of claim 76, wherein the TLR9 agonist is a CpG ODN.

78. The method of claim 75, wherein the TLR agonist is a TLR7 agonist.

79. The method of claim 78, wherein the TLR7 agonist is R848.

80. The method of any of claims 68-79, wherein the FR-targeting agent is an agent capable of binding to a protein of Table 1.

81. The method of any of claims 68-80, wherein the FR-targeting agent is a PRG2-binding agent.

82. The method of claim 81, wherein the PRG2-binding agent is heparin.

83. The method of any of claims 68-80, wherein the FR-targeting agent is a DAP12-binding agent.

84. The method of claim 83, wherein the DAP12-binding agent is a polypeptide comprising SEQ ID NO:1.

85. The method of any of claims 68-80, wherein the FR-targeting agent is a CD206-targeting agent.

86. The method of claim 85, wherein the CD206-targeting agent is a polypeptide comprising SEQ ID NO:2.

87. The method of any of claims 68-80, wherein the FR-targeting agent is a C9orfl35-targeting agent.

88. The method of any of claims 68-80, wherein the FR-targeting agent is a TMEM176A-binding agent.

89. The method of any of claims 68-80, wherein the FR-targeting agent is a TREM2-binding agent.

90. The method of any of claims 68-80, wherein the FR-targeting agent is a CLC5A-binding agent.

91. The method of any of claims 68-90, further comprising an additional FR-targeting agent.

92. The method of claim 91, wherein the FR-targeting agent is a PRG2-binding agent and the additional FR-targeting agent is a DAP12-binding agent.

93. The method of claim 92, wherein the PRG2-binding agent is heparin and the DAP12-binding agent is a polypeptide comprising SEQ ID NO:1.

94. The method of claim 91, wherein the FR-targeting agent is a PRG2-binding agent and the additional FR-targeting agent is a CD206-binding agent.

95. The method of claim 94, wherein the PRG2-binding agent is heparin and the CD206-binding agent is a polypeptide comprising SEQ ID NO:2.

96. The method of claim 91, wherein the FR-targeting agent is a DAP12-binding agent and the additional FR-targeting agent is a CD206-binding agent.

97. The method of claim 96, wherein the DAP12-binding agent is a polypeptide comprising SEQ ID NO:1 and the CD206-binding agent is a polypeptide comprising SEQ ID NO:2.

98. The method of any of claims 68-97, wherein the antigen is a tumor antigen.

99. The method of any of claims 68-98, wherein the subject is a mouse subject.

100. The method of any of claims 68-98, wherein the subject is a human subject.

101. A method for treating or preventing an autoimmune or inflammatory condition in a subject comprising administering to the subject an effective amount of a pharmaceutical composition comprising a therapeutic agent and a First Responder (FR)-targeting agent.

102. The method of claim 101, wherein the therapeutic agent is a cell killing agent.

103. The method of claim 101 or 102, further comprising administering to the subject an additional anti-inflammatory agent.

104. The method of any of claims 101-103, wherein the FR-targeting agent is conjugated to the therapeutic agent.

105. The method of any of claims 101-103, wherein the FR-targeting agent is conjugated to a liposome comprising the therapeutic agent.

106. The method of any of claims 101-103, wherein the FR-targeting agent is conjugated to a nanoparticle comprising the therapeutic agent.

107. The method of any of claims 101-103, wherein the FR-targeting agent is an agent capable of binding to a protein of Table 1.

108. The method of any of claims 101-107, wherein the FR-targeting agent is a PRG2-binding agent.

109. The method of claim 108, wherein the PRG2-binding agent is heparin.

110. The method of any of claims 101-107, wherein the FR-targeting agent is a DAP12-binding agent.

111. The method of claim 110, wherein the DAP12-binding agent is a polypeptide comprising SEQ ID NO:1.

112. The method of any of claims 101-107, wherein the FR-targeting agent is a CD206-targeting agent.

113. The method of claim 112, wherein the CD206-targeting agent is a polypeptide comprising SEQ ID NO:2.

114. The method of any of claims 101-107, wherein the FR-targeting agent is a C9orfl35-targeting agent.

115. The method of any of claims 101-107, wherein the FR-targeting agent is a TMEM176A-binding agent.

116. The method of any of claims 101-107, wherein the FR-targeting agent is a TREM2-binding agent.

117. The method of any of claims 101-107, wherein the FR-targeting agent is a CLC5A-binding agent.

118. The method of any of claims 101-117, further comprising an additional FR-targeting agent.

119. The method of claim 118, wherein the FR-targeting agent is a PRG2-binding agent and the additional FR-targeting agent is a DAP12-binding agent.

120. The method of claim 119, wherein the PRG2-binding agent is heparin and the DAP12-binding agent is a polypeptide comprising SEQ ID NO:1.

121. The method of claim 118, wherein the FR-targeting agent is a PRG2-binding agent and the additional FR-targeting agent is a CD206-binding agent.

122. The method of claim 121, wherein the PRG2-binding agent is heparin and the CD206-binding agent is a polypeptide comprising SEQ ID NO:2.

123. The method of claim 118, wherein the FR-targeting agent is a DAP12-binding agent and the additional FR-targeting agent is a CD206-binding agent.

124. The method of claim 123, wherein the DAP12-binding agent is a polypeptide comprising SEQ ID NO:1 and the CD206-binding agent is a polypeptide comprising SEQ ID NO:2.

125. The method of any of claims 101-124, wherein the subject is a mouse subject.

126. The method of any of claims 101-124, wherein the subject is a human subject.
